(19) United States
(12) Patent Application Publication
(10) Pub. No.: US 2025/0243190 A1
(43) Pub. Date: Jul. 31, 2025

(54) NEW DEUTERATED RORGAMMA/RORGAMMAT INVERSE AGONISTS

(71) Applicant: IMMUNIC AG, Gräfelfing (DE)

(72) Inventors: Christian GEGE, Ehingen (DE); Hella KOHLHOF, München (DE); Andreas MÜHLER, München (DE); Daniel VITT, Germering (DE)

(21) Appl. No.: 18/854,257

(22) PCT Filed: Apr. 14, 2023
(86) PCT No.: PCT/EP2023/059754

§ 371 Date: Oct. 4, 2024

(30) Foreign Application Priority Data
Apr. 14, 2022 (EP) 22168554.8

(51) Int. Cl. C07D 413/14; A61K 31/422; A61K 31/427; A61K 31/506; A61K 31/5377; A61P 31/14; A61P 31/16; C07D 413/04; C07D 417/14

(52) U.S. Cl. CPC C07D 413/14; A61K 31/422; A61K 31/427; A61K 31/506; A61K 31/5377; A61P 31/14; A61P 31/16; C07D 413/04; C07D 417/14

(57) ABSTRACT

The invention relates to novel deuterated compounds of Formula (I)

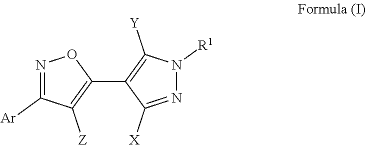

and their use as medicaments.

### SUMMARY OF THE INVENTION

The present disclosure relates to novel RORγ/RORγt inverse agonists according to Formula (I), pharmaceutical formulations comprising them, a process for their preparation and their use as medicament, alone or in combination with one or more additional agents, for treating of various diseases, wherein inverse agonism of is RORγ/RORγt desirable.

### INTRODUCTION AND PRIOR ART

The IL-17 family of cytokines has been associated with the pathogenesis of autoimmune diseases and is generally blamed for the pathogenic symptoms of autoimmune and chronic inflammation. Overexpression of IL-17 is a hallmark of autoimmune and chronic inflammatory diseases like rheumatoid arthritis, psoriasis and psoriatic arthritis, ankylosing spondylitis, inflammatory bowel disease, multiple sclerosis, vasculitis and atherosclerosis, systemic lupus erythematosus, as well as lung disorders, asthma and chronic obstructive pulmonary diseases and diabetes (Nat. Rev. Drug Discov. 2012; 11:763). The IL-17 cytokine family comprises six members, out of which IL-17A and IL-17F are the best characterized. IL-17A and IL-17F are clearly associated with inflammation, whereas the role of the other IL-17 family members is less explored (Cytokine Growth Factor Rev. 2010; 21:413). Secretion of IL-17 is mainly caused by a specific subtype of T helper cells termed T_{H}17 cells. Differentiation of naïve CD4⁺ T cells into T_{H}17 cells is induced in the presence of the cytokines IL-1ß, TGFβ and IL-6, whereas IL-23 maintains T_{H}17 cell survival. The main transcription factor driving the differentiation into TH17 cells is RORγt and is has been shown that inhibiting the activity of RORγt by small molecules or genetically deleting RORgt reduces or inhibits the differentiation into Th17 cells. In addition to driving the differentiation into Th17 cells, RORγt is the main transcription factor for the production of proinflammatory cytokines from the IL-17 family from TH17, innate like T cells including invariant natural killer (iNKT) cells and γδ-T cells (Nat. Commun. 2019; 10; 9—doi.org/10.1038/s41467-018-07911-6). IL-17 itself induces production of effector molecules in IL17R expressing cells like endothelial cells, epithelial cells or fibroblasts, macrophages and dendritic cells, chondrocytes and osteoblasts. Those effector molecules are pro-inflammatory cytokines (IL-6, TNFα and IL-1b), chemokines (like CXCL1, CXCL2, CXCL5, CCL2, CCL7 and CCL20), growth factors (G-CSF, GM-CSF) and nitric oxide, prostaglandin E2 and matrix-metalloproteases. Initiated by these effector molecules, neutrophil infiltration, tissue damage and chronic inflammation occurs (Nat. Rev. Drug Discov. 2012; 11:763) and triggers disease progression by enhancing a pro-inflammatory environment. Before the recognition of the importance of IL-17 in autoimmune inflammation, IFNγ derived from T_{H}1 cells was believed to be the important cytokine that drives autoimmune disorders. IFNγ transcription and secretion from T_{H}1 effector cells is regulated by the transcription factors T-bet and STAT4. As an effector cytokine of T_{H}1 immunity, IFNγ is the key regulator of macrophage activation. In parallel, INFγ signalling generates other cytokines and inflammatory factors to sustain inflammation, maintain T_{H}1 responses and inhibit differentiation of regulatory T cells, T_{H}2 cells and T_{H}17 cells (Discov. Med. 2013; 16:123; J. Biol. Chem. 2017; 292:13925).

Recently, the existence of hybrid T_{H}1/T_{H}17 cells was described. These cells can be induced in vitro by IL-23 and IL-6 in concert with IL-1 and secrete IL-17 and IFNγ. It was demonstrated that these double producing cells harbor pronounced pro-inflammatory properties and are involved in the pathogenesis of inflammatory bowel disease, experimental autoimmune encephalomyelitis and type 1 diabetes. In addition, it has been shown that pathogenic T helper cells exhibit a high plasticity, meaning that IL-17 producing Th17 cells can convert into cells that produce IFNγ (Mediators Inflamm. 2017; 2017:3908061).

The effectiveness of blocking IL-17 signaling alone as therapeutic treatment in autoimmune diseases has already been proven in clinical trials with e.g. monoclonal antibodies against IL-17A (sekukinumab or ixekizumab) and/or the IL-17 receptor IL-17RA (brodalumab). On the other hand, blocking the IFNγ signaling alone in autoimmune diseases with IFNγ-specific monoclonal antibody AMG811 was investigated in clinical trials for systemic and discoid lupus erythematosus without significant clinical benefit so far.

Compounds which target and suppress both IL-17 and IFNγ are therefore predestined for the treatment of autoimmune disorders.

Compounds with the defined deuteration pattern according to Formula (I) have not yet been described in WO2012/101261, WO2012/101263 and WO2019/048541.

### BRIEF DESCRIPTION OF THE DRAWING

FIG. 1 depicts the differences between RORγ/RORγt agonists, antagonists and inverse agonists here differentiated by their different capabilities to recruit coactivators (NcoA) or corepressors (NcoR). The representative graphs can be obtained as readout from the Gal4-assay described in Example 200.

### SUMMARY OF THE INVENTION

The present invention relates to compounds according to Formula (I)

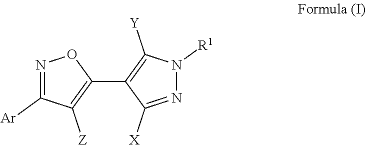

or a pharmaceutically acceptable salt, a solvate, a solvate of a salt, a hydrate or a polymorph thereof, wherein
Ar, X, Y, Z and R¹ are defined as in claim 1,
provided, that at least one hydrogen in Formula (I) is replaced by deuterium with the proviso that the at least one deuterium is not contained in R² and provided, that the level of deuterium incorporation at each substituent designated as deuterium is at least 52.5%.

The compounds of the present invention have a similar or better RORγ/RORγt inverse agonistic activity compared to known RORγ/RORγt inverse agonists. Furthermore, the compounds of the present invention exhibit an advantageous stability or pharmacokinetic profile when used as medicament due to the replacement of hydrogen to deuterium.

Thus, the present invention further relates to a pharmaceutical composition comprising a compound according to Formula (I) and at least one pharmaceutically acceptable carrier or excipient.

The present invention is further directed to compounds according to Formula (I) for use in the prophylaxis and/or treatment of diseases mediated by RORγ/RORγt.

Accordingly, the present invention relates to the prophylaxis and/or treatment of the disease, disorder, therapeutic indication or medical condition which is selected from the group comprising psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behçet's disease, atopic dermatitis. Lichen planus, Sjögren's syndrome, spinal disc herniation, acne, graft-versus-host-reaction, host-versus-graft-reaction, AIH (autoimmune hepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, respiratory syncytial virus (RSV) and influenza, and cancer including leukemia, melanoma, lymphoma, carcinoma and sarcoma, especially prostate cancer including castration-resistant prostate cancer (CRPC).

### DETAILED DESCRIPTION OF THE INVENTION

Surprisingly, we found that by selectively deuterating certain positions, the metabolic stability can be dramatically improved. We also found that compounds according to Formula (I) also show a potent antiviral activity for SARS-CoV-2, RSV and influenza as well.

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

In certain embodiments the present invention relates to a compound of Formula (I) as described in the following items:

1. A compound according to Formula (I):

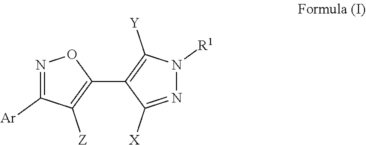

or a pharmaceutically acceptable salt, a solvate, a solvate of a salt, a hydrate or a polymorph thereof, wherein
Ar is selected from the group consisting of phenyl and heteroaryl, each of which is optionally substituted by one to five independently selected substituents R^{Ar}, having one or more hydrogen atoms optionally replaced by deuterium;
R^{Ar} is selected from the group consisting of halogen, —OH, —CN, alkoxy, haloalkoxy, alkyl, haloalkyl, mono- or dialkylamino-alkyl, mono- or di-alkylamino-alkoxy, —COOR′, —CONHR′, —CO—R′, —SO₂NHR′, —NH—CO—R′, —NO₂, —NH—SO₂—R′, —SO₂—R′, benzyloxy, —CO-heterocyclyl, —CO-cycloalkyl, —CONH-cycloalkyl, —CONH-heterocyclyl, —O-alkyl-heterocyclyl, —O-alkyl-cycloalkyl, (2-oxa-6-azaspiro[3.3]hept-6-yl)-C₁₋₄-alkoxy, amino, arylalkyl, cycloalkyl, heterocyclyl, phenyl and heteroaryl, wherein each of said alkoxy, arylalkyl, alkyl, cycloalkyl, heterocyclyl, phenyl and heteroaryl group is optionally substituted by one or more substituents independently selected from alkyl, haloalkyl, halogen and OH, having one or more hydrogen atoms optionally replaced by deuterium;
R′ is independently selected from the group consisting of H, OH, alkyl and haloalkyl, having one or more hydrogen atoms optionally replaced by deuterium;
Z is selected from the group consisting of H, halogen, —CO—R^{Z}, —CH₂—O—R^{Z}, —CO—CH₂—R^{Z}, —CO—CH₂—O—R^{Z}, —COOR^{Z}, —NHCO—R^{Z}, —CO—NHR^{Z}, —N(R^{Z})₂, —CN, —NHCOOR^{Z}, —SO₂—R^{Z}, —SO₂NHR^{Z}, -alkyl-O—R^{Z}, -alkyl-O-alkyl-O—R^{Z}, amino, alkyl, phenyl, heteroaryl, heterocyclyl and cycloalkyl, wherein each of said alkyl, phenyl, heteroaryl, heterocyclyl and cycloalkyl group is optionally substituted by one or more substituents independently selected from the group consisting of halogen, alkyl, alkoxy, haloalkyl, —COO-alkyl, OH and cycloalkyl, having one or more hydrogen atoms optionally replaced by deuterium;
R^{Z} is selected from the group consisting of H, halogen, —OH, alkyl, haloalkyl, cycloalkyl, heterocyclyl, phenyl and heteroaryl, having one or more hydrogen atoms optionally replaced by deuterium;
X is selected from H or D;
Y is selected from H, halogen, haloalkyl, alkyl, cycloalkyl, heterocyclyl or an alkylester, wherein each of said alkyl, cycloalkyl and heterocyclyl is optionally substituted by one or more substituents independently selected from the group consisting of halogen, —OH, —CN, alkyl, alkoxy, haloalkyl and O-haloalkyl, having one or more hydrogen atoms optionally replaced by deuterium;
R¹ is selected from aryl, heteroaryl, cycloalkyl, heterocyclyl or alkyl, which is optionally substituted by one to five substituents R″, having one or more hydrogen atoms optionally replaced by deuterium;
R″ is independently selected from H, —CO₂R′″, —CONHR′″, —CR′″O, —SO₂N(R′″)₂, —SO₂NHR′″, —NR′″—CO-haloalkyl, —NO₂, —NR′″—SO₂-haloalkyl, —NR′″—SO₂-alkyl, —SO₂-alkyl, —NR′″—CO-alkyl, —CN, alkyl, haloalkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl, having one or more hydrogen atoms optionally replaced by deuterium;
R″″ independently represents H, haloalkyl, hydroxyalkyl, amino, alkoxy, —N═C(R″)₂, —NR″—CO—R″, —CR″O, —CO₂R″, alkyl, cycloalkyl, aryl, haloaryl, haloarylalkyl, heteroaryl, heterocyclyl, arylalkyl or aminoalkyl, which are optionally substituted by one or more substituents R″, having one or more hydrogen atoms optionally replaced by deuterium;
wherein the longest chain allowed in R¹ are three coupled substituents R″ and/or R′″, or, alternatively R¹ is a group of the structure

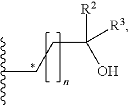

wherein
n is 0 or 1;
R² is H, deuterium, methyl or CD₃;
R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium;
or
n is 1, R² is H, deuterium or methyl and R³ forms a methylene bridge to the carbon atom marked *, having one or more hydrogen atoms optionally replaced by deuterium;
and the hydrogen atom from the carbon atom marked * is substituted by deuterium;
provided, that at least one hydrogen in Formula (I) is replaced by deuterium with the proviso that the at least one deuterium is not contained in R²; and
provided, that the level of deuterium incorporation at each substituent designated as deuterium is at least 52.5%.

2. A compound according to item 1, wherein
Ar is phenyl, which is substituted by one to five independently selected substituents R^{Ar}, having one or more hydrogen atoms optionally replaced by deuterium;
R^{Ar} is selected from the group consisting of F, Cl, Br, —OH, —CN, alkoxy, haloalkoxy, alkyl, haloalkyl, —O-alkyl-heterocyclyl, —O-alkyl-cycloalkyl, having one or more hydrogen atoms optionally replaced by deuterium;
X is H.

3. A compound according to item 1 or 2, wherein
Y is selected from haloalkyl, alkyl, cycloalkyl or heterocyclyl, wherein each of said alkyl, cycloalkyl and heterocyclyl is optionally substituted by one or more substituents independently selected from the group consisting of halogen, —OH, CN, alkyl, alkoxy, haloalkyl and O-haloalkyl, having one or more hydrogen atoms optionally replaced by deuterium.

4. A compound according to item 1 to 3, wherein
Z is selected from the group consisting of —CO—R^{Z}, —CH₂—O—R^{Z}, —CO—CH₂—R^{Z}, —CO—CH₂—O—R^{Z}, —COOR^{Z}, -alkyl-O—R^{Z}, alkyl, phenyl, heteroaryl, heterocyclyl and cycloalkyl, wherein each of said alkyl, phenyl, heteroaryl, heterocyclyl and cycloalkyl group is optionally substituted by one or more substituents independently selected from the group consisting of halogen, alkyl, alkoxy, haloalkyl, —COO-alkyl, OH and cycloalkyl, having one or more hydrogen atoms optionally replaced by deuterium;
R^{Z} is selected from the group consisting of H, halogen, —OH, alkyl, haloalkyl, cycloalkyl, heterocyclyl, phenyl and heteroaryl, having one or more hydrogen atoms optionally replaced by deuterium.

5. A compound according to item 1 to 4, wherein
R¹ is a group of the structure

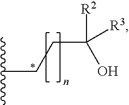

wherein
n is 0 or 1;
R² is H, deuterium, methyl or CD₃;
R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium;
or
n is 1, R² is H, deuterium or methyl and R³ forms a methylene bridge to the carbon atom marked *, having one or more hydrogen atoms optionally replaced by deuterium;
and the hydrogen atom from the carbon atom marked * is substituted by deuterium.

6. A compound according to item 1 to 5, which is selected from

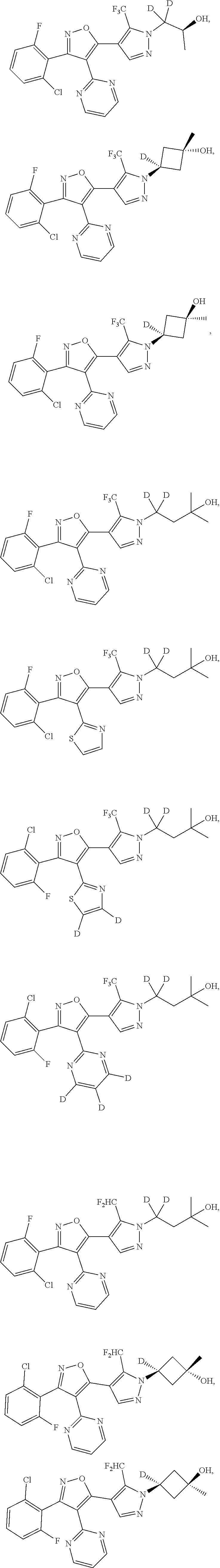

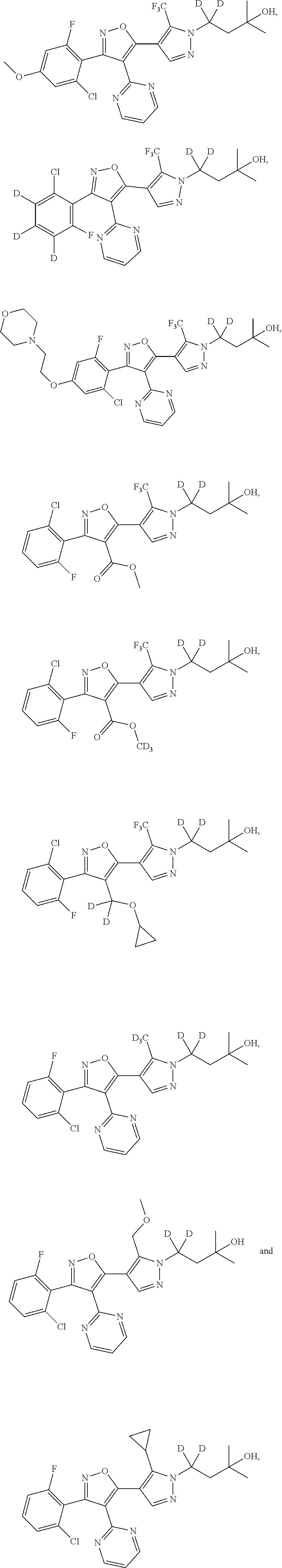

or a pharmaceutically acceptable salt, a solvate, a solvate of a salt, a hydrate or a polymorph thereof.

7. A compound according to item 1 to 4, wherein
R¹ is aryl or heteroaryl, which is optionally substituted by one to five substituents selected from the group consisting of F, Cl, Br, CN, —OH, alkyl, haloalkyl, —O-alkyl and —O-haloalkyl, having one or more hydrogen atoms optionally replaced by deuterium.

8. A compound according to item 1 to 4 or item 7, which is selected from, which is selected from

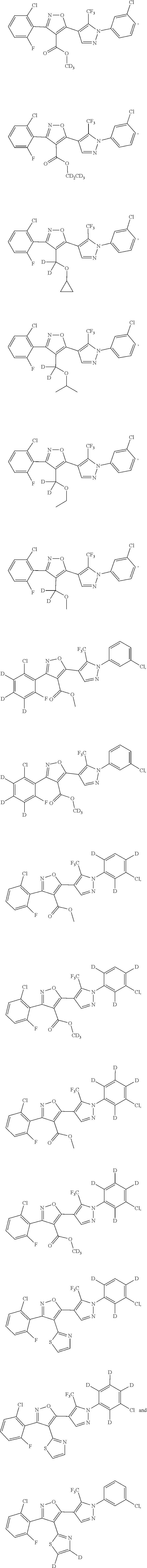

or a pharmaceutically acceptable salt, a solvate, a solvate of a salt, a hydrate or a polymorph thereof.

9. The compound according to item 1 to 8 for use as a medicament.

10. The compound according to item 1 to 8 for use in the treatment of a disease or medical condition is selected from the group consisting of psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behcet disease, atopic dermatitis, Lichen planus, Sjögren's syndrome, spinal disc herniation, acne, graft-versus-host-reaction, host-versus-graft-reaction, AIH (autoimmunhepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, RSV and influenza, and cancer including leukemia, melanoma, lymphoma, carcinoma and sarcoma, especially prostate cancer including castration-resistant prostate cancer (CRPC).

11. Use of a compound of the Formula (I) as defined in item 1 to 8, or a pharmaceutically acceptable salt or solvate thereof in the manufacture of a medicament for the treatment of a disease or medical condition in which inverse agonism of RORγ/RORγt and/or inhibition of interleukin-17 (IL-17) and/or Interferon-γ (INF-γ) is beneficial.

12. The use of item 11 wherein the disease or medical condition is selected from the group consisting of psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behçet's disease, atopic dermatitis. Lichen planus, Sjögren's syndrome, spinal disc herniation, acne, graft-versus-host-reaction, host-versus-graft-reaction, AIH (autoimmune hepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, RSV and influenza, and cancer including leukemia, melanoma, lymphoma, carcinoma and sarcoma, especially prostate cancer including castration-resistant prostate cancer (CRPC).

13. A pharmaceutical composition comprising a compound according item 1 to 8 and a pharmaceutically acceptable carrier or excipient.

### DEFINITIONS AND PARTICULAR EMBODIMENTS

In particular embodiments, as used herein, wherein when any one of the substituent is deuterium, the level of deuterium incorporation at each of substituent designated as deuterium is at least 50.1%, at least 52.5%, at least 75%, at least 82.5%, at least 90%, at least 95%, at least 97%, at least 98%, or at least 99%.

In particular embodiments, the level of deuterium incorporation at each substituent designated as deuterium is at least 50.1%.

In particular embodiments, the level of deuterium incorporation at each substituent designated as deuterium is at least 52.5%.

More particularly, the level of deuterium incorporation at each substituent designated as deuterium is at least 90%. Even more particularly, the level of deuterium incorporation at each substituent designated as deuterium is at least 95%. Most particularly, the level of deuterium incorporation at each substituent designated as deuterium is at least 98%.

Quantitative analysis of specifically deuterated compounds can be achieved by a number of conventional methods, such as mass spectroscopy (peak area) or by quantifying the remaining residual ¹H-NMR signals of the specific deuteration site compared to signals from internal standards or other, non-deuterated ¹H signals in the compound.

It will be recognized that some variation of natural isotopic abundance occurs in a synthesized compound depending upon the origin of chemical materials used in the synthesis. Thus, a preparation of a not-specifically deuterated compound will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Comp. Biochem. Physiol. 1998; 119A:725. The term “isotopic enrichment factor” at a particular position normally occupied by hydrogen refers to the ratio between the abundance of deuterium at the position and the natural abundance of deuterium at that position. By way of example, an isotopic enrichment factor of 3500 means that the amount of deuterium at the particular position is 3500-fold the natural abundance of deuterium, or that 52.5% of the compounds have deuterium at the particular position (i.e., 52.5% deuterium incorporation at the given position). The abundance of deuterium in the oceans of Earth is approximately one atom in 6500 hydrogen atoms (about 154 parts per million (ppm)). Deuterium thus accounts for approximately 0.015 percent (on a weight basis, 0.030 percent) of all naturally occurring hydrogen atoms in the oceans on Earth; the abundance changes slightly from one kind of natural water to another.

When a particular position in a compound of the invention (e.g., a compound represented by Formula (I) or a pharmaceutically acceptable salt and/or solvate thereof) is designated by name or structure as containing hydrogen or deuterium, it is to be understood that the position can contain hydrogen at its natural abundance or can be enriched in deuterium with an isotopic enrichment factor of, for example, at least 835 (12.5% deuterium incorporation), of at least 1670 (25% deuterium incorporation, of at least 3500 (52.5% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When a particular position in a compound of the invention (e.g., a compound represented by Formula (I) or a pharmaceutically acceptable salt and/or solvate thereof) is designated specifically by name or structure as “H” or “hydrogen”, the position is understood to have hydrogen at its natural abundance isotopic composition.

When a particular position in a compound of the invention (e.g., a compound represented by Formula (I) or a pharmaceutically acceptable salt and/or solvate thereof) is designated specifically by name or structure as “D” or “deuterium”, the position is understood to have deuterium at an abundance that is at least 3340 times of the natural abundance of deuterium, which is 0.015% (i.e., at least 50.1% incorporation of deuterium), at least 3500 times of the natural abundance of deuterium (52.5% deuterium incorporation), at least 4500 times of the natural abundance of deuterium (67.5% deuterium incorporation), at least 5000 (75% deuterium), at least 5500 times of the natural abundance of deuterium (82.5% deuterium incorporation), at least 6000 times of the natural abundance of deuterium (90% deuterium incorporation), at least 6333.3 times of the natural abundance of deuterium (95% deuterium incorporation), at least 6466.7 times of the natural abundance of deuterium (97% deuterium incorporation), at least 6600 times of the natural abundance of deuterium (99% deuterium incorporation), or at least 6633.3 times of the natural abundance of deuterium (99.5% deuterium incorporation).

The percentage of deuterium incorporation can be obtained by quantitative analysis using a number of conventional methods, such as mass spectroscopy (peak area) or by quantifying the remaining residual ¹H-NMR signals of the specific deuteration site compared to signals from internal standards or other, non-deuterated ¹H signals in the compound.

When a chemical name or structure is silent as to whether a particular position in a compound normally occupied by hydrogen is isotopically enriched, it is intended that the particular position is occupied by hydrogen at its natural abundance. By way of example, the term “phenyl” or

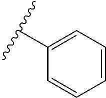 [SMILES: *c1ccccc1]

without any further designation as to isotopic enrichment indicates that all hydrogen atoms are present at natural abundance.

The term “compound,” when referring to any compound of this disclosure, including a compound represented by Formula (I) or a pharmaceutically acceptable salt and/or solvate thereof, refers to a collection of molecules having an identical chemical structure, except that there may be isotopic variation among the constituent hydrogen atoms of the molecules. The relative amount of isotopic variation in a compound of this invention will depend upon a number of factors including the isotopic purity of deuterated reagents used to make the compound and the efficiency of incorporation of deuterium in the various synthesis steps used to prepare the compound.

“D” and “d” both refer to deuterium. “H” refers to hydrogen.

“Substituted with deuterium” refers to the replacement of one or more hydrogen atoms with a corresponding number of deuterium atoms.

Any formula or structure given herein, is also intended to represent deuterated compounds comprising in addition further isotopically labelled atoms. Examples of additional isotopes that can be incorporated into compounds of the disclosure include further isotopes of hydrogen (i.e. tritium or ³H), as well as isotopes of carbon, nitrogen, oxygen, phosphorous, fluorine and chlorine, such as, but not limited to ¹¹C, ¹³C, ¹⁴C, ¹⁵N, ¹⁸F, ³¹P, ³²P, ³⁵, ³⁶Cl and ¹²⁵I. The disclosure further comprises various isotopically labelled compounds into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or radioactive treatment of patients.

The compounds of the present invention act as RORγ/RORγt modulators, preferably as RORγ/RORγt inverse agonists.

Ligands to nuclear receptors including RORγ/RORγt ligands can either act as agonists, antagonists or inverse agonists. An agonist in this context means a small molecule ligand that binds to the receptor and stimulates its transcriptional activity as determined by e.g. an increase of mRNAs or proteins that are transcribed under control of an RORγ/RORγt response element. Transcriptional activity can also be determined in biochemical or cellular in vitro assays that employ just the ligand binding domain of RORγ/RORγt but use the interaction with a cofactor (i.e. a corepressor “NcoR” or a coactivator “NcoA”), potentially in conjunction with a generic DNA-binding element such as the Gal4 domain, to monitor agonistic, antagonistic or inverse agonistic activity.

Whereas an agonist by this definition stimulates RORγ/RORγt- or RORγ/RORγt-Gal4-driven transcriptional activity, an antagonist is defined as a small molecule that binds to RORγ/RORγt and thereby inhibits transcriptional activation that would otherwise occur through an endogenous RORγ/RORγt ligand.

An inverse agonist differs from an antagonist in that it not only binds to RORγ/RORγt and inhibits transcriptional activity but in that it actively shuts down transcription directed by RORγ/RORγt, even in the absence of an endogenous agonist. Whereas it is difficult to differentiate between RORγ/RORγt antagonistic and inverse agonistic activity in vivo, given that there are always some levels of endogenous RORγ/RORγt agonist present, biochemical or cellular reporter assays can more clearly distinguish between the two activities. At a molecular level an inverse agonist does not allow for the recruitment of a coactivator protein or active parts thereof whereas it should lead to an active recruitment of corepressor proteins are active parts thereof. An RORγ/RORγt antagonist in this context would be defined as an RORγ/RORγt ligand that neither leads to coactivator nor to corepressor recruitment but acts just through displacing RORγ/RORγt agonists. Therefore, the use of assays such as the Gal4-mammalian-two-hybrid assay is mandatory in order to differentiate between coactivator or corepressor-recruiting RORγ/RORγt compounds (Nat. Rev. Drug Discov. 2004; 3:950).

Since the boundaries between RORγ/RORγt agonists, RORγ/RORγt antagonists and RORγ/RORγt inverse agonists are not sharp but fluent, the term “RORγ/RORγt modulator” was coined to encompass all compounds which are not clean RORγ/RORγt agonists but show a certain degree of corepressor recruitment in conjunction with a reduced RORγ/RORγt transcriptional activity. RORγ/RORγt modulators therefore encompass RORγ/RORγt antagonists and RORγ/RORγt inverse agonists and it should be noted that even a weak RORγ/RORγt agonist can act as an RORγ/RORγt antagonist if it prevents a full agonist from full transcriptional activation.

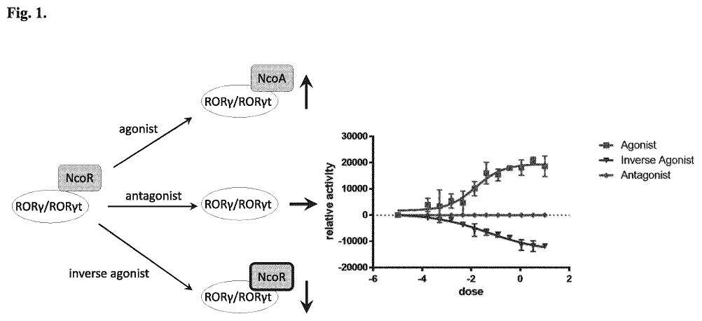

The differences between RORγ/RORγt agonists, antagonists and inverse agonists here differentiated by their different capabilities to recruit coactivators (NcoA) or corepressors (NcoR). The representative graphs can be obtained as readout from the Gal4-assay described in Example 200 (see FIG. 1).

In particular embodiments, as used herein a heteroaryl group denotes a 5- or 6-membered heterocyclic group having aromatic character containing one to four heteroatoms independently selected from O, N or S. This heterocyclic group is optionally fused to another aromatic or heteroaromatic 5- or 6-membered ring containing from one to four heteroatoms independently selected from O, N or S. For example, this group can be selected from a thiadiazolyl, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, 1,2,4-oxadiazol-3-yl, 1,2,4-oxadiazol-5-yl, 1,2,5-oxadiazol-3-yl, benzooxazol-2-yl, benzooxazol-4-yl, benzooxazol-5-yl, benzoisooxazol-3-yl, benzoisooxazol-4-yl, benzoisooxazol-5-yl, 1,2,5-oxadiazol-4-yl, 1,3,4-oxadiazol-2-yl, 1,2,4-thiadiazol-3-yl, 1,2,4-thiadiazol-5-yl, 1,3,4-thiadiazol-2-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, pyrid-2-yl, pyrid-3-yl, pyrid-4-yl, pyrid-5-yl, pyrid-6-yl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1H-tetrazol-2-yl, 1H-tetrazol-3-yl, tetrazolyl, acridyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazanyl, benzothiofurazanyl, benzotriazol-1-yl, benzotriazol-4-yl, benzotriazol-5-yl, benzotriazol-6-yl, benzotriazol-7-yl, benzotriazinyl, benzo[b]thiophenyl, benzimidazolyl, benzothiazolyl, quinazolinyl, quinoxazolinyl, cinnolinyl, quinolinyl, tetrahydroquinolinyl, isoquinolinyl, or tetrahydroisoquinolinyl, purinyl, phthalazinyl, pteridinyl, thiatetraazaindenyl, thiatriazaindenyl, isothiazolopyrazinyl, 6-pyrimidinyl, 2,4-dimethoxy-6-pyrimidinyl, benzimidazol-2-yl, 1H-benzimidazolyl, benzimidazol-4-yl, benz-imidazol-5-yl, benzimidazol-6-yl, benzimidazol-7-yl, tetrazolyl, tetrahydro-thieno[3,4-d]imidazol-2-one, pyrazolo[5,1-c][1,2,4]triazine, isothiazolopyrimidine, pyrazolotriazine, pyrazolopyrimidine, imidazopyridazine, imidazopyrimidine, imidazopyridine, imidazolotriazine, triazolotriazine, triazolopyridine, triazolopyrazine, triazolopyrimidine, or triazolopyridazine group. Particular heteroaryl groups are pyrimidin-4-yl, pyrimidin-2-yl, thiazol-2-yl, pyrazin-2-yl and isoxazol-2-yl. More particular heteroaryl groups are pyrimidin-2-yl and thiazol-2-yl. One or more hydrogen atoms in the heteroaryl group is optionally replaced by deuterium.

In particular embodiments, as used herein a heterocyclyl group denotes a 3- to 8-membered, more particularly a 3 to 6-membered cyclic non-aromatic group containing from one to four heteroatoms independently selected from the group consisting of O, N, and S, wherein the heterocyclyl group is optionally fused to another non-aromatic cycloalkyl or heterocyclyl ring; the heterocyclyl residue is in particular selected from the group consisting of oxetanyl, morpholine-4-yl, piperazinyl, isoxazolidine-2-yl, 1-alkylpiperazine-4-yl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl and pyranyl. Particular heterocyclyl groups are tetrahydropyranyl and oxetanyl. One or more hydrogen atoms in the heterocyclyl group is optionally replaced by deuterium.

To keep the definitions as short as possible, as used herein “alkyl”, and “alk” (as e.g. in alkoxy) is to be understood to encompass linear and branched alkanyl, alkenyl and alkynyl, more particularly alkanyl and alkenyl, even more particularly alkanyl. If not stated otherwise, these are in particular embodiments C₁₋₆-alkanyl, C₂₋₆-alkenyl or C₂₋₆-alkynyl, more particularly C₁₋₅-alkanyl, C₂₋₅-alkenyl or C₂₋₅-alkynyl, even more particularly C₁₋₄-alkanyl, C₂₋₄-alkenyl or C₂₋₄-alkynyl. The alkyl group may for instance be selected from the group consisting of —CH₃, —C₂H₅, —CH═CH₂, —C≡CH, —C₃H₇, —CH(CH₃)₂, —CH₂—CH═CH₂, —C(CH₃)═CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, —C(CH₃)═C(CH₃)₂, —C₃H₆—C≡CH, —C≡C—C₃H₇, —C₂H₄—C≡C—CH₃, —CH₂—C═C≡C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇ and —C₂H₄—C≡C—C₂H₅. Particular alkyl groups are methyl, ethyl, propyl, isopropyl, butyl, tert-butyl, pentyl, butenyl and pentenyl. One or more hydrogen atoms in the alkyl group is optionally replaced by deuterium.

For example, the term “having one or more hydrogen atoms optionally replaced by deuterium” in relation to “C₁₋₄-alkyl” encompasses (but not limited to) the following residues: CD₃, CH₂D, CHD₂, CD₃CH₂(CH₂)ₙ, CD₃CH₂(CHD)ₙ, CD₃CH₂(CD₂)ₙ, CH₂DCH₂(CH₂)ₙ, CH₂DCH₂(CHD)ₙ, CH₂DCH₂(CD₂)ₙ, CHD₂CH₂(CH₂)ₙ, CHD₂CH₂(CHD)ₙ, CHD₂CH₂(CD₂)ₙ, CD₃CHD(CH₂)ₙ, CD₃CHD(CHD)ₙ, CD₃CHD(CD₂)ₙ, CH₂DCHD(CH₂)ₙ, CH₂DCHD(CHD)ₙ, CH₂DCHD(CD₂)ₙ, CHD₂CHD(CH₂)ₙ, CHD₂CHD(CHD)ₙ, CHD₂CHD(CD₂)ₙ, CH₃CHD(CH₂)ₙ, CH₃CHD(CHD)ₙ, CH₃CHD(CD₂)ₙ, CD₃CD₂(CH₂)ₙ, CD₃CD₂(CHD)ₙ, CD₃CD₂(CD₂)ₙ, CH₂DCD₂(CH₂)ₙ, CH₂DCD₂(CHD)ₙ, CH₂DCD₂(CD₂)ₙ, CHD₂CD₂(CH₂)ₙ, CHD₂CD₂(CHD)ₙ, CHD₂CD₂(CD₂)ₙ, CH₃CD₂(CH₂)ₙ, CH₃CD₂(CHD)ₙ, CH₃CD₂(CD₂)ₙ, wherein n is an integer from 0 to 2, and CH₃CH₂(CHD)ₘ, CH₃CH₂(CD₂)ₘ, wherein m is an integer from 1 to 2, as well as CD(CD₃)₂, CH(CD₃)₂ and C(CD₃)₃. Preferred C₁₋₂-alkyl containing deuterium are CD₃ and CD₃CD₂, most preferred is CD₃. In particular embodiments, as used herein a cycloalkyl group denotes a non-aromatic ring system containing three to eight carbon atoms, wherein each of the atoms forming the ring is a carbon atom, particularly four to eight carbon atoms, more particularly three to six carbon atoms, even more particularly three to five carbon atoms.

Similar, the term “C₃₋₄-cycloalkyl” having one or more hydrogen atoms in alkyl optionally replaced by deuterium” encompasses, but is not limited to the following residues:

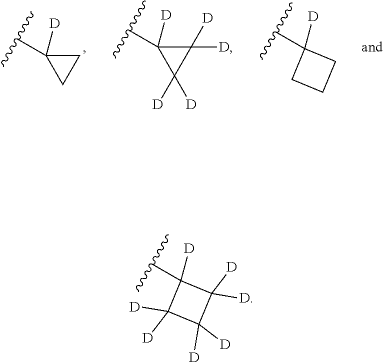

A cycloalkyl or heterocyclyl group can be connected straight or spirocyclic, e.g. when cyclohexane is substituted with the heterocycloalkyl group oxetane, the following structures are possible:

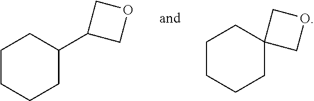 [SMILES: C1CCC(C2COC2)CC1 ; C1CCC2(CC1)COC2]

The heterocyclyl group can be connected with the remaining part of the molecule via a carbon, nitrogen (e.g. in morpholine or piperidine) or sulfur atom. An example for a S-linked heterocycloalkyl is the cyclic sulfonimidamide

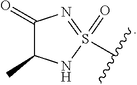 [SMILES: *S1(=O)=NC(=O)[C@H](C)N1]

In particular embodiments, as used herein an arylalkyl group denotes a linear or branched C₁-C₆-alkyl substituted with at least one aryl group as defined herein. Exemplary arylalkyl groups include benzyl, phenylethyl, 4-hydroxybenzyl, 3-fluorobenzyl, 2-fluorophenylethyl and the like.

In particular embodiments, as used herein an alkoxy group denotes an O-alkyl group, the alkyl group being as defined above. More particularly the alkoxy group is a methoxy, ethoxy, isopropoxy, tert-butoxy or pentoxy group, even more particularly methoxy.

In particular embodiments, as used herein a haloalkyl group denotes an alkyl group wherein one or more, particularly more than half, more particularly all, of the hydrogen atoms are replaced by halogen atoms.

The haloalkyl group is for instance —C(R¹⁰)₃, —CR¹⁰(R^{10′})₂, —CR¹⁰(R^{10′})R^{10″}, —C₂(R¹⁰)₅, —CH₂—C(R¹⁰)₃, —C(R^{10′})₂—CH(R^{10′})₂, —CH₂—CR¹⁰(R^{10′})₂, —CH₂—CR¹⁰(R^{10′})R^{10″}, —C₃(R¹⁰)₇, or —C₂H₄—C(R¹⁰)₃, wherein R¹⁰, R^{10′}, R^{10″} particularly independently represent F, Cl, Br or I, more particularly F. More particularly, haloalkyl is CF₂CF₃, CHF₂ or CF₃. Most particularly, haloalkyl is CF₃.

In particular embodiments, as used herein a haloalkoxy group denotes an —O-haloalkyl group.

In particular embodiments, as used herein a halo or halogen group denotes fluorine, chlorine, bromine or iodine; particularly chlorine or fluorine.

Unless stated otherwise, the terms “included”, “including”, “include” and the like are to be understood as meaning including but non-limiting.

Constituents which are optionally substituted as stated herein may be substituted, unless otherwise noted, at any chemically possible position.

In particular embodiments of the present invention, Ar is selected from the group consisting of phenyl and heteroaryl, each of which is optionally substituted by one to five substituents R′ independently selected from the group consisting of halogen, —OH, —CN, alkoxy, haloalkoxy, alkyl, haloalkyl, mono- or dialkylamino-alkyl, mono- or di-alkylamino-alkoxy, —COOR′, —CONHR′, —CO—R′, —SO₂NHR′, —NH—CO—R′, —NO₂, —NH—SO₂—R′, —SO₂—R′, benzyloxy, —CO-heterocyclyl, —CO-cycloalkyl, —CONH-cycloalkyl, —CONH-heterocyclyl, —O-alkyl-heterocyclyl, —O-alkyl-cycloalkyl, (2-oxa-6-azaspiro[3.3]hept-6-yl)-C₁-4-alkoxy, amino, arylalkyl, cycloalkyl, heterocyclyl, phenyl and heteroaryl, wherein each of said alkoxy, arylalkyl, alkyl, cycloalkyl, heterocyclyl, phenyl and heteroaryl group is optionally substituted by one or more substituents independently selected from alkyl, haloalkyl, halogen and OH, having one or more hydrogen atoms in Ar, R^{Ar} or R′ optionally replaced by deuterium;
in other particular embodiments Ar is phenyl, which is optionally substituted by one to five substituents independently selected from F, Cl, Br, —OH, —CN, alkoxy, haloalkoxy, alkyl, haloalkyl, —O-alkyl-heterocyclyl, —O-alkyl-cycloalkyl, having one or more hydrogen atoms in Ar or its substituent(s) optionally replaced by deuterium;
in other particular embodiments Ar is phenyl, which is optionally substituted by one to five substituents independently selected from fluoro, chloro, OH, —OMe, —OCHF₂, —OCF₃, —CO-heterocyclyl and —O—C₁₋₂-alkyl-heterocyclyl, having one or more hydrogen atoms in Ar or its substituent(s) optionally replaced by deuterium;
in other particular embodiments Ar is phenyl, which is optionally substituted by one to five substituents independently selected from D, fluoro, chloro, OH, —OCH₃, —OCD₃, —OCHF₂, —OCDF₂, —OCF₃, —CO— heterocyclyl and —O—C₁₋₂-alkyl-heterocyclyl;
in other particular embodiments Ar is selected from

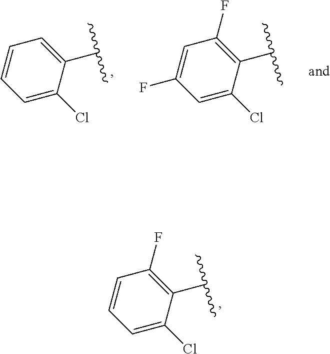 [SMILES: *c1ccccc1Cl ; *c1c(F)cc(F)cc1Cl ; *c1c(F)cccc1Cl]

having one or more hydrogen atoms optionally replaced by deuterium;
in other particular embodiments Ar is selected from

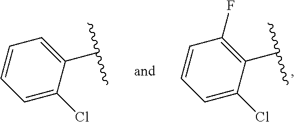 [SMILES: *c1ccccc1Cl ; *c1c(F)cccc1Cl]

having one or more hydrogen atoms optionally replaced by deuterium;
in other particular embodiments Ar is selected from

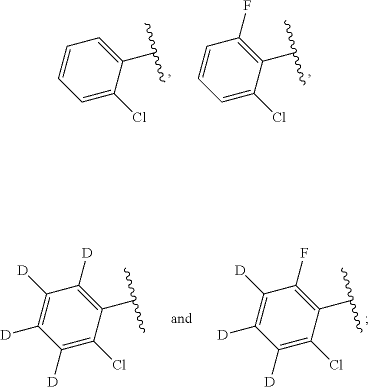 [SMILES: *c1ccccc1Cl ; *c1c(F)cccc1Cl ; *c1c([2H])c([2H])c([2H])c([2H])c1Cl ; *c1c(F)c([2H])c([2H])c([2H])c1Cl]

in other particular embodiments Ar is selected from

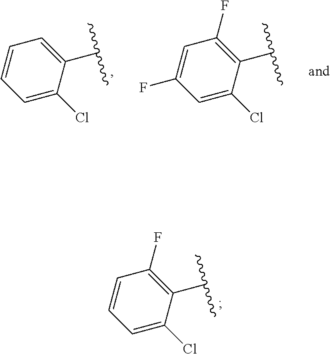 [SMILES: *c1ccccc1Cl ; *c1c(F)cc(F)cc1Cl ; *c1c(F)cccc1Cl]

in other particular embodiments Ar is selected from

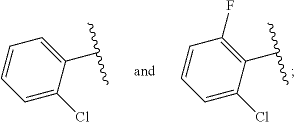 [SMILES: *c1ccccc1Cl ; *c1c(F)cccc1Cl]

in other particular embodiments Ar is

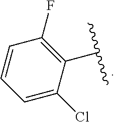 [SMILES: *c1c(F)cccc1Cl]

In particular embodiments of the present invention, the compound of Formula (I) is deuterated
(a) in alpha position to the pyrazole when R¹ is selected from the group consisting of alkyl or cycloalkyl, optionally substituted by one to five substituents R″;
(b) in alpha position to the oxazole when Z is selected from the group consisting of —CH₂—O—R^{Z}, -alkyl-O—R^{Z}, alkyl, phenyl, heterocyclyl and cycloalkyl, wherein each of said alkyl, heterocyclyl and cycloalkyl group is optionally substituted by one or more substituents independently selected from the group consisting of halogen, alkyl, alkoxy, haloalkyl, —COO-alkyl, OH and cycloalkyl, having one or more hydrogen atoms optionally replaced by deuterium; R^{Z} is selected from the group consisting of H, halogen, —OH, alkyl, haloalkyl, cycloalkyl, heterocyclyl, phenyl and heteroaryl, having one or more hydrogen atoms optionally replaced by deuterium;
(c) in R^{Z} when Z is —COOR^{Z}, and R^{Z} is selected from the group consisting of alkyl, haloalkyl and cycloalkyl.

In particular embodiments of the present invention, Y is selected from H, halogen, haloalkyl, alkyl, cycloalkyl, heterocyclyl or an alkylester, wherein each of said alkyl, cycloalkyl and heterocyclyl is optionally substituted by one or more substituents independently selected from the group consisting of halogen, —OH, —CN, alkyl, alkoxy, haloalkyl and O-haloalkyl, having one or more hydrogen atoms optionally replaced by deuterium;
in other particular embodiments Y is selected from haloalkyl, alkyl, cycloalkyl or heterocyclyl, wherein each of said alkyl, cycloalkyl and heterocyclyl is optionally substituted by one or more substituents independently selected from the group consisting of halogen, —OH, CN, alkyl, alkoxy, haloalkyl and O-haloalkyl, having one or more hydrogen atoms optionally replaced by deuterium;
in other particular embodiments Y is selected from alkyl, fluoroalkyl, cycloalkyl and —CH₂—O-alkyl, having one or more hydrogen atoms optionally replaced by deuterium;
in other particular embodiments Y is selected from CH₃, CD₃, CHF₂, CDF₂ and CF₃;
in other particular embodiments Y is selected from CH₃, CD₃, CHF₂ and CF₃;
in other particular embodiments Y is CF₃.

In particular embodiments of the present invention, Z is selected from the group consisting of H, halogen, —CO—R^{Z}, —CH₂—O—R^{Z}, —CO—CH₂—R^{Z}, —CO—CH₂—O—R^{Z}, —COOR^{Z}, —NHCO—R^{Z}, —CO—NHR^{Z}, —N(R^{Z})₂, —CN, —NHCOOR^{Z}, —SO₂—R^{Z}, —SO₂NHR^{Z}, -alkyl-O—R^{Z}, -alkyl-O-alkyl-O—R^{Z}, amino, alkyl, phenyl, heteroaryl, heterocyclyl and cycloalkyl, wherein each of said alkyl, phenyl, heteroaryl, heterocyclyl and cycloalkyl group is optionally substituted by one or more substituents independently selected from the group consisting of halogen, alkyl, alkoxy, haloalkyl, —COO-alkyl, OH and cycloalkyl, and R^{Z} is selected from the group consisting of H, halogen, —OH, alkyl, haloalkyl, cycloalkyl, heterocyclyl, phenyl and heteroaryl, having one or more hydrogen atoms in Z or R^{Z} optionally replaced by deuterium;
in other particular embodiments Z is selected from the group consisting of —CO—R^{Z}, —CH₂—O—R^{Z}, —CO—CH₂—R^{Z}, —CO—CH₂—O—R^{Z}, —COOR^{Z}, -alkyl-O—R^{Z}, alkyl, phenyl, heteroaryl, heterocyclyl and cycloalkyl, wherein each of said alkyl, phenyl, heteroaryl, heterocyclyl and cycloalkyl group is optionally substituted by one or more substituents independently selected from the group consisting of halogen, alkyl, alkoxy, haloalkyl, —COO-alkyl, OH and cycloalkyl, and R^{Z} is selected from the group consisting of H, halogen, —OH, alkyl, haloalkyl, cycloalkyl, heterocyclyl, phenyl and heteroaryl, having one or more hydrogen atoms in Z or R^{Z} optionally replaced by deuterium;
in other particular embodiments Z is selected from the group consisting of —CO—C₁₋₃-alkyl, —CH₂—O—C₃₋₄-cycloalkyl, thiazolyl and pyrimidyl, having one or more hydrogen atoms optionally replaced by deuterium;
in other particular embodiments Z is selected from

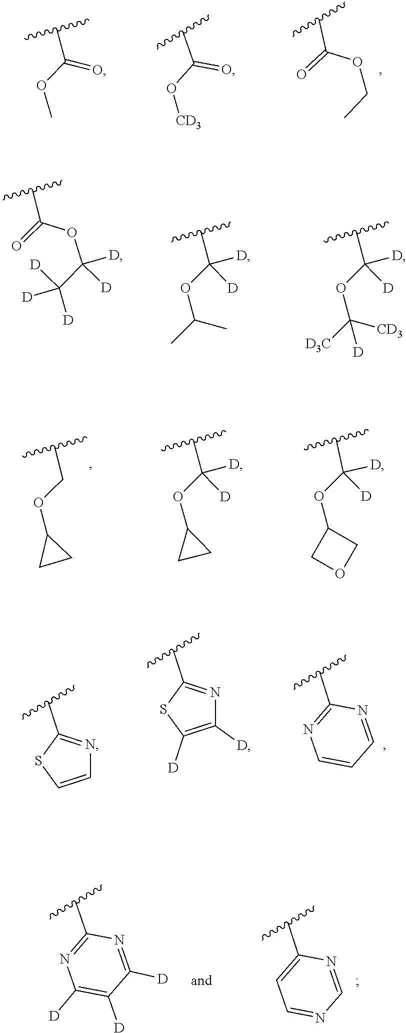

in other particular embodiments Z is selected from

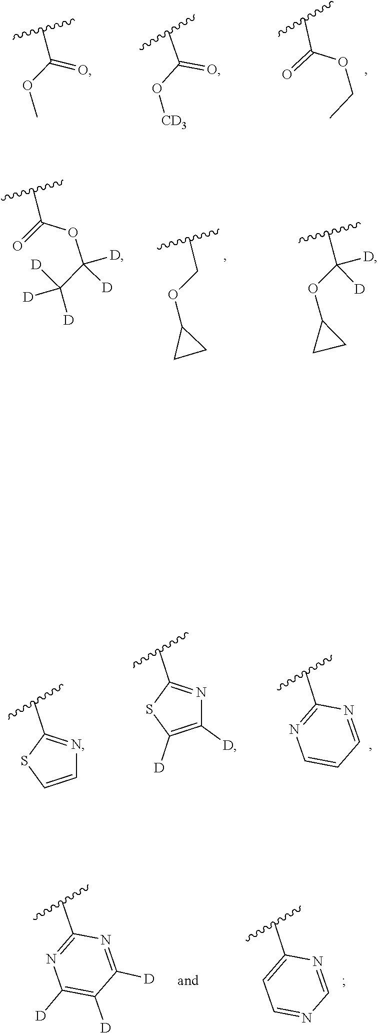

in other particular embodiments Z is selected from

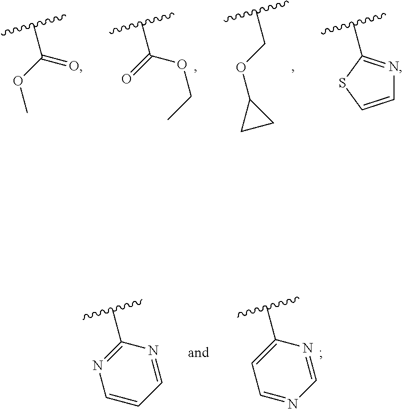

in other particular embodiments Z is selected from

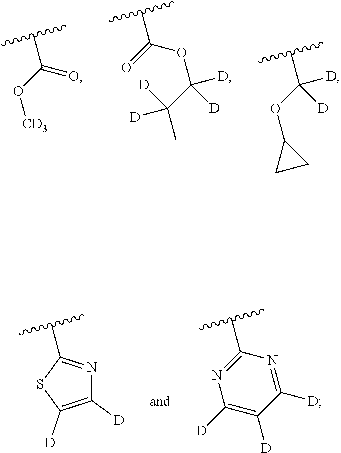

in other particular embodiments Z is

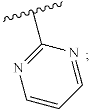 [SMILES: *c1ncccn1]

in other particular embodiments Z is

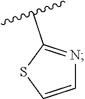 [SMILES: *c1nccs1]

in other particular embodiments Z is

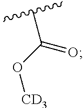 [SMILES: *C(=O)OC([2H])([2H])[2H]]

in other particular embodiments Z is

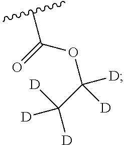

in other particular embodiments Z is

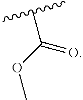 [SMILES: *C(=O)OC]

In particular embodiments of the present invention, Z is selected from

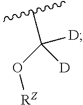

R^{Z} is selected from the group consisting of alkyl, haloalkyl, cycloalkyl and heterocyclyl, having one or more hydrogen atoms in R^{Z} optionally replaced by deuterium;
in other particular embodiments Z is selected from

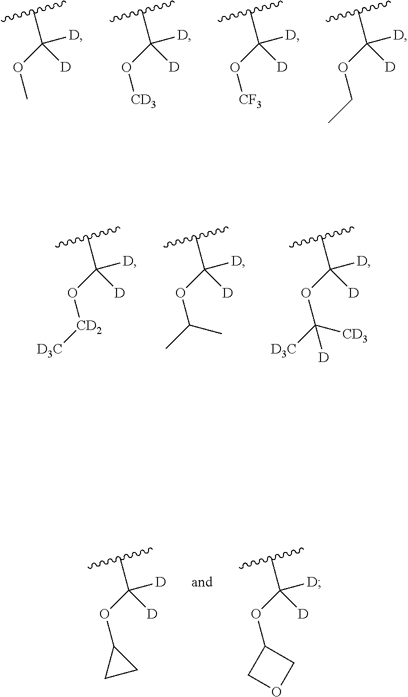

in other particular embodiments Z is selected from

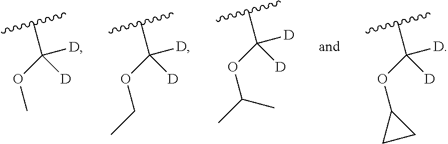

In particular embodiments of the present invention, R¹ is selected from aryl, heteroaryl, cycloalkyl, heterocyclyl or alkyl, which is optionally substituted by one to five substituents R″, having one or more hydrogen atoms optionally replaced by deuterium, and R″ is independently selected from H, —CO₂R′″, —CONHR′″, —CR′″O, —SO₂N(R′″)₂, —SO₂NHR′″, —NR′″—CO-haloalkyl, —NO₂, —NR′″—SO₂-haloalkyl, —NR′″—SO₂-alkyl, —SO₂-alkyl, —NR′″—CO-alkyl, —CN, alkyl, haloalkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl, having one or more hydrogen atoms optionally replaced by deuterium, and R′″ independently represents H, haloalkyl, hydroxyalkyl, amino, alkoxy, —N═C(R″)₂, —NR″—CO—R″, —CR″O, —CO₂R″, alkyl, cycloalkyl, aryl, haloaryl, haloarylalkyl, heteroaryl, heterocyclyl, arylalkyl or aminoalkyl, which are optionally substituted by one or more substituents R″, having one or more hydrogen atoms optionally replaced by deuterium, wherein the longest chain allowed in R¹ are three coupled substituents R″ and/or R′″,
or, alternatively R¹ is a group of the structure

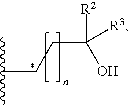

wherein
n is 0 or 1, and R² is H, deuterium, methyl, CD₃, and R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium, or n is 1, R² is H, deuterium or methyl and R³ forms a methylene bridge to the carbon atom marked *, having one or more hydrogen atoms optionally replaced by deuterium, and the hydrogen atom from the carbon atom marked * is substituted by deuterium, provided, that at least one hydrogen in Formula (I) is replaced by deuterium with the proviso that the at least one deuterium is not contained in R²;
in other particular embodiments R¹ is a group of the structure

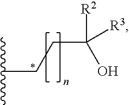

wherein
n is 0 or 1, and R² is H, deuterium, methyl, CD₃, and R³ is methyl, trifluoromethyl, ethyl, or taken with R² together forms a cyclopropyl group, having one or more hydrogen atoms optionally replaced by deuterium, and the hydrogen atoms from the carbon atom marked * are substituted by deuterium, or n is 1, R² is H, deuterium or methyl and R³ forms a methylene bridge to the carbon atom marked *, having one or more hydrogen atoms optionally replaced by deuterium, and the hydrogen atom from the carbon atom marked * is substituted by deuterium;
in other particular embodiments R¹ is selected from

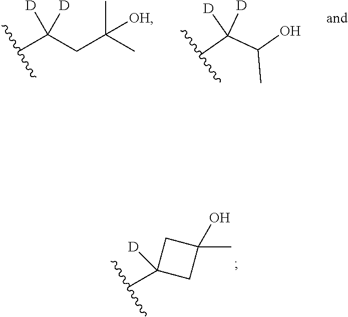

in other particular embodiments R¹ is selected from

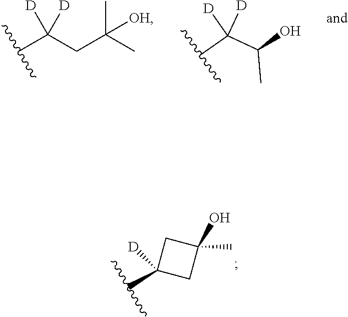

in other particular embodiments R¹ is

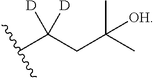 [SMILES: *C([2H])([2H])CC(C)(C)O]

In yet other particular embodiments R¹ is selected from aryl or heteroaryl, which is optionally substituted by one to five substituents selected from the group consisting of F, Cl, Br, CN, —OH, alkyl, haloalkyl, —O-alkyl and —O-haloalkyl, having one or more hydrogen atoms optionally replaced by deuterium;
in other particular embodiments R¹ is phenyl which is optionally substituted by one to five substituents selected from the group consisting of F, Cl, Br, CN, —OH, alkyl, haloalkyl, —O-alkyl and —O-haloalkyl, having one or more hydrogen atoms optionally replaced by deuterium;
in other particular embodiments R¹ is phenyl which is substituted by one to five substituents selected from the group consisting of F, Cl and Me, having one or more hydrogen atoms optionally replaced by deuterium;
in other particular embodiments R¹ is phenyl which is substituted by Cl, having one or more hydrogen atoms optionally replaced by deuterium;
in other particular embodiments R¹ is selected from

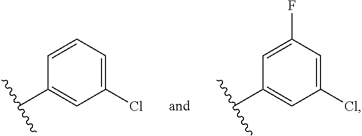 [SMILES: *c1cccc(Cl)c1 ; *c1cc(F)cc(Cl)c1]

having one or more hydrogen atoms optionally replaced by deuterium;
in other particular embodiments R¹ is

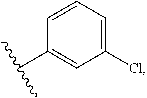 [SMILES: *c1cccc(Cl)c1]

having one or more hydrogen atoms optionally replaced by deuterium;
in other particular embodiments R¹ is selected from

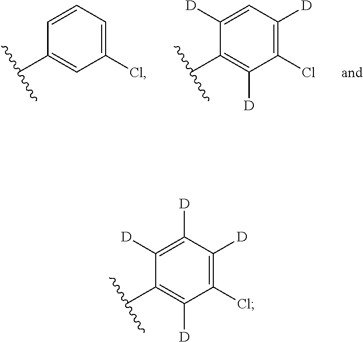 [SMILES: *c1cccc(Cl)c1 ; *c1c([2H])cc([2H])c(Cl)c1[2H] ; *c1c([2H])c([2H])c([2H])c(Cl)c1[2H]]

in other particular embodiments R¹ is

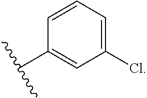 [SMILES: *c1cccc(Cl)c1]

In particular embodiments of the present invention, X is selected from H or D;
in other particular embodiments X is H.

In one embodiment in a compound according to Formula (I):
Ar is phenyl, which is substituted by one to five independently selected substituents R^{Ar}, having one or more hydrogen atoms optionally replaced by deuterium;
R^{Ar} is selected from the group consisting of F, Cl, Br, —OH, —CN, alkoxy, haloalkoxy, alkyl, haloalkyl, —O-alkyl-heterocyclyl, and —O-alkyl-cycloalkyl, having one or more hydrogen atoms optionally replaced by deuterium;
X is H or D;
Y is selected from haloalkyl, alkyl, cycloalkyl or heterocyclyl, wherein each of said alkyl, cycloalkyl and heterocyclyl is optionally substituted by one or more substituents independently selected from the group consisting of halogen, —OH, CN, alkyl, alkoxy, haloalkyl and O-haloalkyl, having one or more hydrogen atoms optionally replaced by deuterium;
Z is selected from the group consisting of —CO—R^{Z}, —CH₂—O—R^{Z}, —CO—CH₂—R^{Z}, —CO—CH₂—O—R^{Z}, —COOR^{Z}, -alkyl-O—R^{Z}, alkyl, phenyl, heteroaryl, heterocyclyl and cycloalkyl, wherein each of said alkyl, phenyl, heteroaryl, heterocyclyl and cycloalkyl group is optionally substituted by one or more substituents independently selected from the group consisting of halogen, alkyl, alkoxy, haloalkyl, —COO-alkyl, OH and cycloalkyl, having one or more hydrogen atoms optionally replaced by deuterium;
R^{Z} is selected from the group consisting of H, halogen, —OH, alkyl, haloalkyl, cycloalkyl, heterocyclyl, phenyl and heteroaryl, having one or more hydrogen atoms optionally replaced by deuterium;
R¹ is a group of the structure

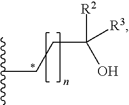

Wherein
n is 0 or 1;
R² is H, deuterium, methyl or CD₃;
R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium;
or
n is 1, R² is H, deuterium or methyl and R³ forms a methylene bridge to the carbon atom marked *, having one or more hydrogen atoms optionally replaced by deuterium;
and the hydrogen atom from the carbon atom marked * is substituted by deuterium.

In one embodiment in a compound according to Formula (I):
Ar is phenyl, which is substituted by one to three independently selected substituents R^{Ar}, having one or more hydrogen atoms optionally replaced by deuterium;
R^{Ar} is selected from the group consisting of F, Cl, Br, alkoxy, alkyl, —O-alkyl-heterocyclyl, and —O-alkyl-cycloalkyl, having one or more hydrogen atoms optionally replaced by deuterium;
X is H or D;
Y is selected from haloalkyl, alkyl, cycloalkyl or heterocyclyl, wherein said alkyl, is optionally substituted by one or more substituents independently selected from the group consisting of halogen, or alkoxy, having one or more hydrogen atoms optionally replaced by deuterium;
Z is selected from the group consisting of —CO—R^{Z}, —CH₂—O—R^{Z}, —CO—CH₂—R^{Z}, —CO—CH₂—O—R^{Z}, —COOR^{Z}, -alkyl-O—R^{Z}, and heteroaryl, having one or more hydrogen atoms optionally replaced by deuterium;
R^{Z} is selected from the group consisting of H, halogen, —OH, cycloalkyl and alkyl, having one or more hydrogen atoms optionally replaced by deuterium;
R¹ is a group of the structure

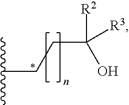

wherein
n is 0 or 1;
R² is H, deuterium, methyl or CD₃;
R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium;
or
n is 1, R² is H, deuterium or methyl and R³ forms a methylene bridge to the carbon atom marked *, having one or more hydrogen atoms optionally replaced by deuterium;
and the hydrogen atom from the carbon atom marked * is substituted by deuterium.

In one embodiment in a compound according to Formula (I):
Ar is phenyl, which is substituted by one to three independently selected substituents R^{Ar}, having one or more hydrogen atoms optionally replaced by deuterium;
R^{Ar} is selected from the group consisting of F, Cl, Br, and —O-alkyl-heterocyclyl;
X is H;
Y is selected from haloalkyl, alkyl, or cycloalkyl, wherein said alkyl is optionally substituted by one or more alkoxy groups, having one or more hydrogen atoms optionally replaced by deuterium;
Z is selected from the group consisting of —CH₂—O—R^{Z}, —COOR^{Z}, -alkyl-O—R^{Z}, and heteroaryl, having one or more hydrogen atoms optionally replaced by deuterium;
R^{Z} is selected from the group consisting of H, cycloalkyl and alkyl, having one or more hydrogen atoms optionally replaced by deuterium;
R¹ is a group of the structure

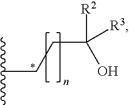

wherein
n is 0 or 1;
R² is H, deuterium, methyl or CD₃;
R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium;
or
n is 1, R² is H, deuterium or methyl and R³ forms a methylene bridge to the carbon atom marked *, having one or more hydrogen atoms optionally replaced by deuterium;
and the hydrogen atom from the carbon atom marked * is substituted by deuterium.

In one embodiment in a compound according to Formula (I):
Ar is phenyl, which is substituted by one to three independently selected substituents R^{Ar}, having one or more hydrogen atoms optionally replaced by deuterium;
R^{Ar} is selected from the group consisting of F, Cl, and —O-alkyl-morpholinyl;
X is H;
Y is selected from the group consisting of CF₃, CHF₂, CD₃, CH₂OCH₃ and cycloalkyl;
Z is selected from the group consisting of —CH₂—O—R^{Z}, —COOR^{Z}, and pirimidyl, having one or more hydrogen atoms optionally replaced by deuterium;
R^{Z} is selected from the group consisting of H, cycloalkyl and alkyl, having one or more hydrogen atoms optionally replaced by deuterium;
R¹ is a group of the structure

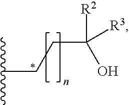

wherein
n is 0 or 1;
R² is H, deuterium, methyl or CD₃;
R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium;
or
n is 1, R² is H, deuterium or methyl and R³ forms a methylene bridge to the carbon atom marked *, having one or more hydrogen atoms optionally replaced by deuterium;
and the hydrogen atom from the carbon atom marked * is substituted by deuterium.

In one embodiment in a compound according to Formula (I):
Ar is phenyl, which is substituted by one to three independently selected substituents R^{Ar}, having one or more hydrogen atoms optionally replaced by deuterium;
R^{Ar} is selected from the group consisting of F, Cl, and —O-alkyl-morpholinyl;
X is H;
Y is selected from the group consisting of CF₃, CHF₂, CD₃, CH₂OCH₃ and cycloalkyl;
Z is selected from the group consisting of —CH₂—O—R^{Z}, —COOR^{Z}, and pirimidyl, having one or more hydrogen atoms optionally replaced by deuterium;
R^{Z} is selected from the group consisting of H, cycloalkyl and alkyl, having one or more hydrogen atoms optionally replaced by deuterium;
R¹ is selected from

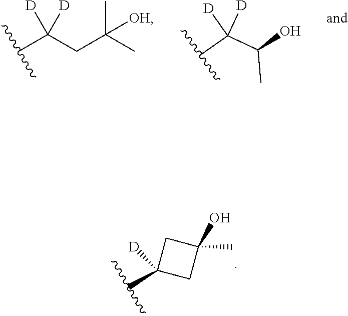

Particular compounds of the present invention are the compounds of the below examples of the present invention.

Particular compounds of the present invention are selected from

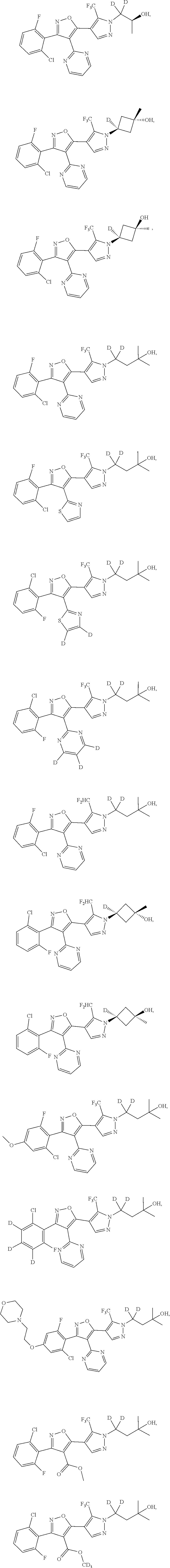

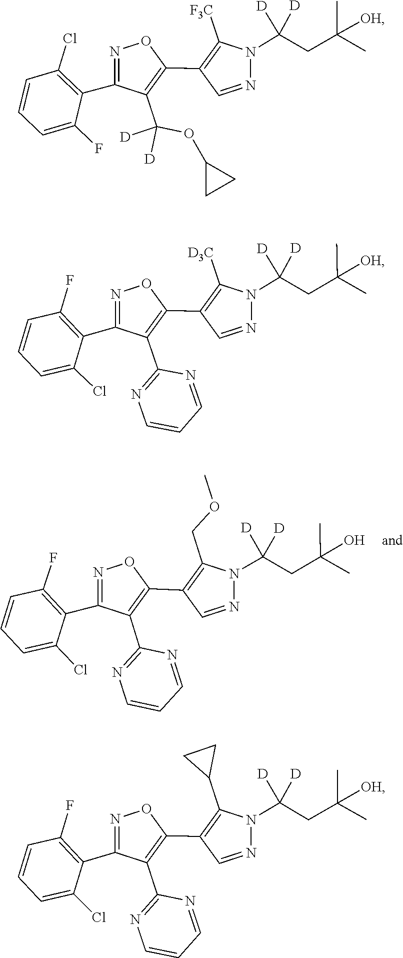 [SMILES: [2H]C([2H])(OC1CC1)c1c(-c2c(F)cccc2Cl)noc1-c1cnn(C([2H])([2H])CC(C)(C)O)c1C(F)(F)F ; [2H]C([2H])([2H])c1c(-c2onc(-c3c(F)cccc3Cl)c2-c2ncccn2)cnn1C([2H])([2H])CC(C)(C)O ; [2H]C([2H])(CC(C)(C)O)n1ncc(-c2onc(-c3c(F)cccc3Cl)c2-c2ncccn2)c1COC ; [2H]C([2H])(CC(C)(C)O)n1ncc(-c2onc(-c3c(F)cccc3Cl)c2-c2ncccn2)c1C1CC1]

or a pharmaceutically acceptable salt, a solvate, a solvate of a salt, a hydrate or a polymorph thereof.

In one embodiment in a compound according to Formula (I):
Ar is phenyl, which is substituted by one to five independently selected substituents R^{Ar}, having one or more hydrogen atoms optionally replaced by deuterium;
R^{Ar} is selected from the group consisting of F, Cl, Br, —OH, —CN, alkoxy, haloalkoxy, alkyl, haloalkyl, —O-alkyl-heterocyclyl, and —O-alkyl-cycloalkyl, having one or more hydrogen atoms optionally replaced by deuterium;
X is H or D;
Y is selected from haloalkyl, alkyl, cycloalkyl or heterocyclyl, wherein each of said alkyl, cycloalkyl and heterocyclyl is optionally substituted by one or more substituents independently selected from the group consisting of halogen, —OH, CN, alkyl, alkoxy, haloalkyl and O-haloalkyl, having one or more hydrogen atoms optionally replaced by deuterium;
Z is selected from the group consisting of —CO—R^{Z}, —CH₂—O—R^{Z}, —CO—CH₂—R^{Z}, —CO—CH₂—O—R^{Z}, —COOR^{Z}, -alkyl-O—R^{Z}, alkyl, phenyl, heteroaryl, heterocyclyl and cycloalkyl, wherein each of said alkyl, phenyl, heteroaryl, heterocyclyl and cycloalkyl group is optionally substituted by one or more substituents independently selected from the group consisting of halogen, alkyl, alkoxy, haloalkyl, —COO-alkyl, OH and cycloalkyl, having one or more hydrogen atoms optionally replaced by deuterium;
R^{Z} is selected from the group consisting of H, halogen, —OH, alkyl, haloalkyl, cycloalkyl, heterocyclyl, phenyl and heteroaryl, having one or more hydrogen atoms optionally replaced by deuterium;
R¹ is selected from aryl, heteroaryl, cycloalkyl, heterocyclyl or alkyl, which is optionally substituted by one to five substituents R″, having one or more hydrogen atoms optionally replaced by deuterium; R″ is independently selected from H, —CO₂R′″, —CONHR′″, —CR′″O, —SO₂N(R′″)₂, —SO₂NHR′″, —NR′″—CO-haloalkyl, —NO₂, —NR′″—SO₂-haloalkyl, —NR′″—SO₂-alkyl, —SO₂-alkyl, —NR′″—CO-alkyl, —CN, alkyl, haloalkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl, having one or more hydrogen atoms optionally replaced by deuterium;

In one embodiment in a compound according to Formula (I):
Ar is phenyl, which is substituted by one to three independently selected substituents R^{Ar}, having one or more hydrogen atoms optionally replaced by deuterium; R^{Ar} is selected from the group consisting of F, Cl, Br, alkoxy, alkyl, —O-alkyl-heterocyclyl, and —O-alkyl-cycloalkyl, having one or more hydrogen atoms optionally replaced by deuterium;
X is H or D;
Y is selected from haloalkyl, alkyl, cycloalkyl or heterocyclyl, wherein said alkyl, is optionally substituted by one or more substituents independently selected from the group consisting of halogen, or alkoxy, having one or more hydrogen atoms optionally replaced by deuterium;
Z is selected from the group consisting of —CO—R^{Z}, —CH₂—O—R^{Z}, —CO—CH₂—R^{Z}, —CO—CH₂—O—R^{Z}, —COOR^{Z}, -alkyl-O—R^{Z}, and heteroaryl, having one or more hydrogen atoms optionally replaced by deuterium;
R^{Z} is selected from the group consisting of H, halogen, —OH, cycloalkyl and alkyl, having one or more hydrogen atoms optionally replaced by deuterium;
R¹ is selected from aryl or heteroaryl, which is optionally substituted by one to five substituents selected from the group consisting of F, Cl, Br, CN, —OH, alkyl, haloalkyl, —O-alkyl and —O-haloalkyl, having one or more hydrogen atoms optionally replaced by deuterium.

In one embodiment in a compound according to Formula (I):
Ar is phenyl, which is substituted by one to three independently selected substituents R^{Ar}, having one or more hydrogen atoms optionally replaced by deuterium;
R^{Ar} is selected from the group consisting of F, Cl, Br, and —O-alkyl-heterocyclyl;
X is H;
Y is selected from haloalkyl, alkyl, or cycloalkyl, wherein said alkyl is optionally substituted by one or more alkoxy groups, having one or more hydrogen atoms optionally replaced by deuterium;
Z is selected from the group consisting of —CH₂—O—R^{Z}, —COOR^{Z}, -alkyl-O—R^{Z}, and heteroaryl, having one or more hydrogen atoms optionally replaced by deuterium;
R^{Z} is selected from the group consisting of H, cycloalkyl and alkyl, having one or more hydrogen atoms optionally replaced by deuterium;
R¹ is selected from

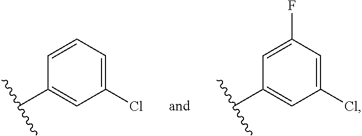 [SMILES: *c1cccc(Cl)c1 ; *c1cc(F)cc(Cl)c1]

having one or more hydrogen atoms optionally replaced by deuterium.

In one embodiment in a compound according to Formula (I):
Ar is phenyl, which is substituted by one to three independently selected substituents R^{Ar}, having one or more hydrogen atoms optionally replaced by deuterium;
R^{Ar} is selected from the group consisting of F, Cl, and —O-alkyl-morpholinyl;
X is H;
Y is selected from the group consisting of CF₃, CHF₂, CD₃, CH₂OCH₃ and cycloalkyl;
Z is selected from the group consisting of —CH₂—O—R^{Z}, —COOR^{Z}, and pirimidyl, having one or more hydrogen atoms optionally replaced by deuterium;
R^{Z} is selected from the group consisting of H, cycloalkyl and alkyl, having one or more hydrogen atoms optionally replaced by deuterium;
R¹ is

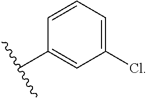 [SMILES: *c1cccc(Cl)c1]

Other particular compounds of the present invention are selected from

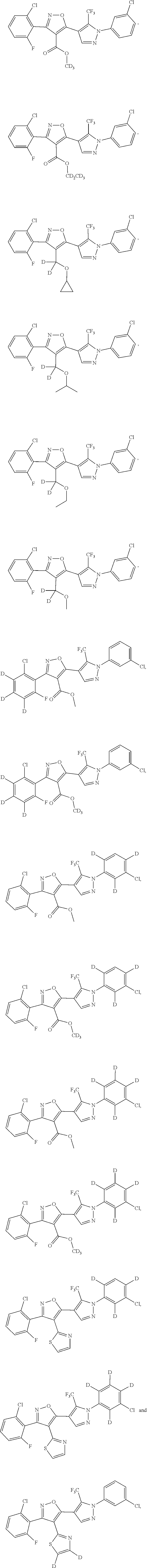

or a pharmaceutically acceptable salt, a solvate, a solvate of a salt, a hydrate or a polymorph thereof. Other particular compounds of the present invention are selected from

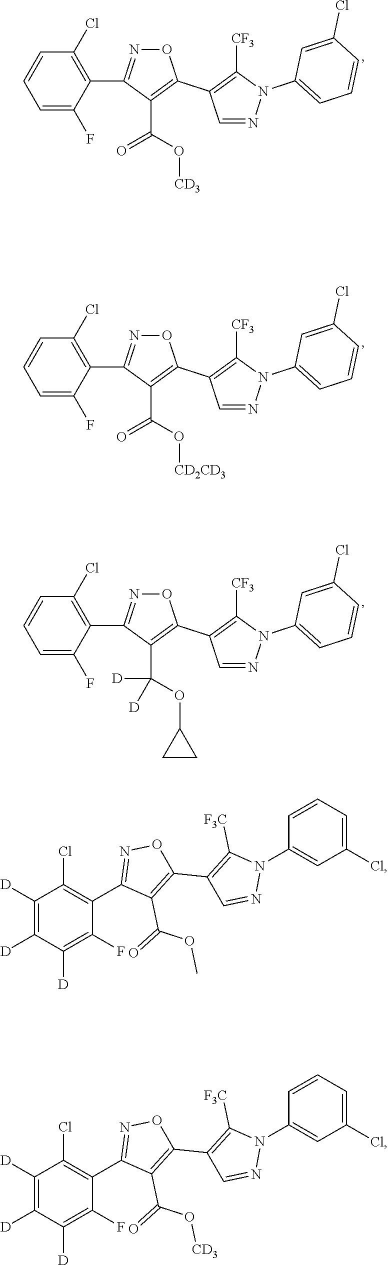

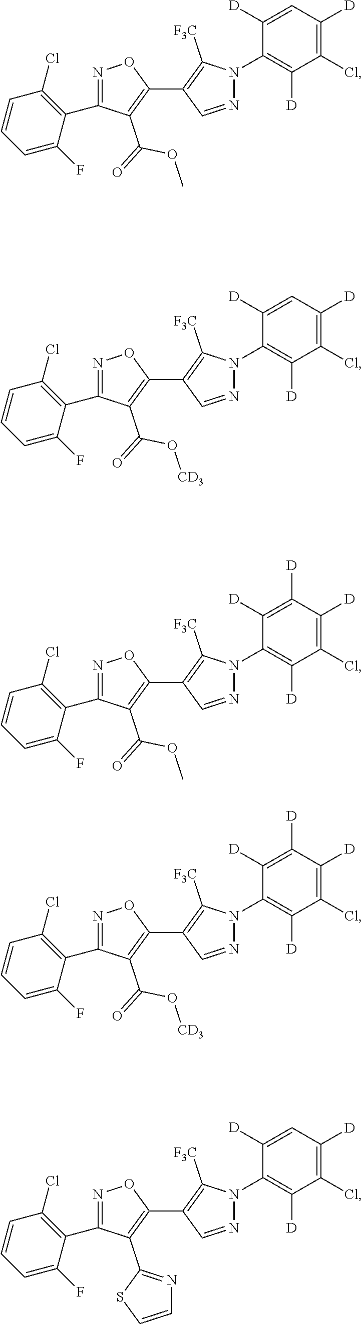 [SMILES: [2H]c1cc([2H])c(-n2ncc(-c3onc(-c4c(F)cccc4Cl)c3C(=O)OC)c2C(F)(F)F)c([2H])c1Cl ; [2H]c1cc([2H])c(-n2ncc(-c3onc(-c4c(F)cccc4Cl)c3C(=O)OC([2H])([2H])[2H])c2C(F)(F)F)c([2H])c1Cl ; [2H]c1c([2H])c(Cl)c([2H])c(-n2ncc(-c3onc(-c4c(F)cccc4Cl)c3C(=O)OC)c2C(F)(F)F)c1[2H] ; [2H]c1c([2H])c(Cl)c([2H])c(-n2ncc(-c3onc(-c4c(F)cccc4Cl)c3C(=O)OC([2H])([2H])[2H])c2C(F)(F)F)c1[2H] ; [2H]c1cc([2H])c(-n2ncc(-c3onc(-c4c(F)cccc4Cl)c3-c3nccs3)c2C(F)(F)F)c([2H])c1Cl]

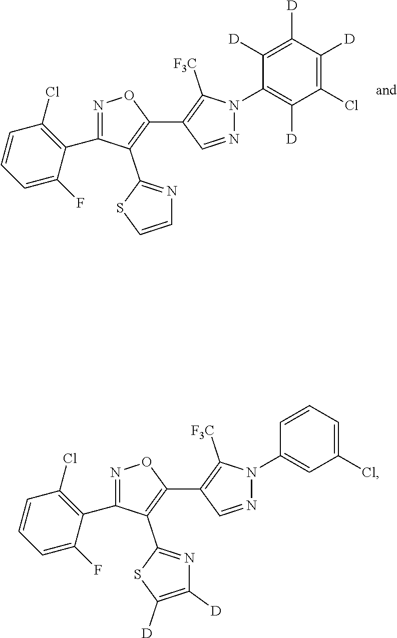 [SMILES: [2H]c1c([2H])c(Cl)c([2H])c(-n2ncc(-c3onc(-c4c(F)cccc4Cl)c3-c3nccs3)c2C(F)(F)F)c1[2H] ; [2H]c1nc(-c2c(-c3c(F)cccc3Cl)noc2-c2cnn(-c3cccc(Cl)c3)c2C(F)(F)F)sc1[2H]]

or a pharmaceutically acceptable salt, a solvate, a solvate of a salt, a hydrate or a polymorph thereof. More particular compounds of the present invention are selected from

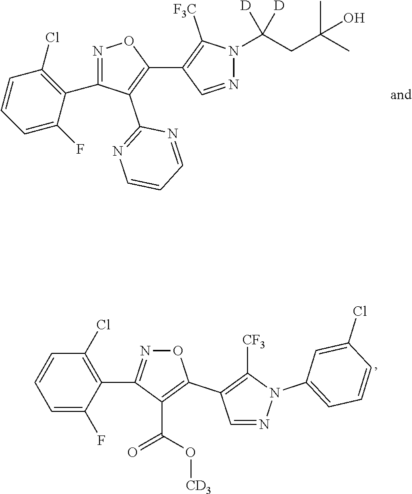 [SMILES: [2H]C([2H])(CC(C)(C)O)n1ncc(-c2onc(-c3c(F)cccc3Cl)c2-c2ncccn2)c1C(F)(F)F ; [2H]C([2H])([2H])OC(=O)c1c(-c2c(F)cccc2Cl)noc1-c1cnn(-c2cccc(Cl)c2)c1C(F)(F)F]

or a pharmaceutically acceptable salt, a solvate, a solvate of a salt, a hydrate or a polymorph thereof. More particular compounds of the present invention are selected from

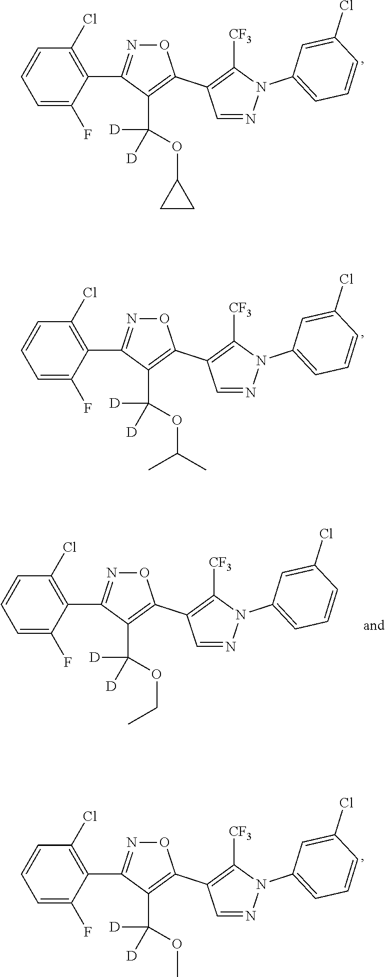 [SMILES: [2H]C([2H])(OC1CC1)c1c(-c2c(F)cccc2Cl)noc1-c1cnn(-c2cccc(Cl)c2)c1C(F)(F)F ; [2H]C([2H])(OC(C)C)c1c(-c2c(F)cccc2Cl)noc1-c1cnn(-c2cccc(Cl)c2)c1C(F)(F)F ; [2H]C([2H])(OCC)c1c(-c2c(F)cccc2Cl)noc1-c1cnn(-c2cccc(Cl)c2)c1C(F)(F)F ; [2H]C([2H])(OC)c1c(-c2c(F)cccc2Cl)noc1-c1cnn(-c2cccc(Cl)c2)c1C(F)(F)F]

or a pharmaceutically acceptable salt, a solvate, a solvate of a salt, a hydrate or a polymorph thereof. The invention also provides the compound of the present invention for the use as a medicament. Also provided is the compound of the present invention for use in the treatment of a disease or medical condition is selected from the group consisting of psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behçet's disease, atopic dermatitis, Lichen planus, Sjögren's syndrome, spinal disc herniation, acne, graft-versus-host-reaction, host-versus-graft-reaction, AIH (autoimmune hepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, RSV and influenza, and cancer including leukemia, melanoma, lymphoma, carcinoma and sarcoma, especially prostate cancer including castration-resistant prostate cancer (CRPC).

In one embodiment the compound of the present invention for use in the treatment of a disease or medical condition is selected from the group consisting of psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behçet's disease, atopic dermatitis, Lichen planus, Sjögren's syndrome, spinal disc herniation, acne, graft-versus-host-reaction, host-versus-graft-reaction, AIH (autoimmune hepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, and influenza, and cancer including leukemia, melanoma, lymphoma, carcinoma and sarcoma, especially prostate cancer including castration-resistant prostate cancer (CRPC).

More specifically, the invention relates to a compound of the present invention for use in the treatment of a disease or medical condition is selected from the group consisting of psoriasis, psoriatic arthritis, infectious viral diseases including Covid-19, RSV and influenza, and cancer including leukemia, melanoma, lymphoma, carcinoma and sarcoma, especially prostate cancer including castration-resistant prostate cancer (CRPC).

In one embodiment, the invention relates to a compound of the present invention for use in the treatment of a disease or medical condition is selected from the group consisting of psoriasis, psoriatic arthritis, infectious viral diseases including Covid-19, and influenza, and cancer including leukemia, melanoma, lymphoma, carcinoma and sarcoma, especially prostate cancer including castration-resistant prostate cancer (CRPC).

Even more specifically, the invention relates to a compound of the present invention for use in the treatment of a disease or medical condition is selected from the group consisting of psoriasis, psoriatic arthritis, Covid-19, influenza, and castration-resistant prostate cancer (CRPC).

Also provides is the compound of the present invention in the manufacture of a medicament for the treatment of a disease or medical condition in which inverse agonism of RORγ/RORγt and/or inhibition of interleukin-17 (IL-17) and/or Interferon-γ (INF-γ) is beneficial.

More specifically, the compound of the present invention in the manufacture of a medicament for the treatment of a disease or medical condition in which inverse agonism of RORγ/RORγt is beneficial.

Also provided is the compound of the present invention in the manufacture of a medicament for the treatment of a disease or medical condition in which inverse agonism of RORγ/RORγt and/or inhibition of interleukin-17 (IL-17) and/or Interferon-γ (INF-γ) is beneficial, wherein the disease or medical condition is selected from the group consisting of psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behçet's disease, atopic dermatitis. Lichen planus, Sjögren's syndrome, spinal disc herniation, acne, graft-versus-host-reaction, host-versus-graft-reaction, AIH (autoimmune hepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, RSV and influenza, and cancer including leukemia, melanoma, lymphoma, carcinoma and sarcoma, especially prostate cancer including castration-resistant prostate cancer (CRPC).

Also provided is a pharmaceutical composition comprising a compound of the present invention and a pharmaceutically acceptable carrier or excipient.

The pharmaceutical compositions as described herein comprise one or more of the compounds according to this invention and a pharmaceutically acceptable carrier or excipient, further comprising one or more additional therapeutic agents selected from anti-inflammatory agents, immunosuppressive and/or immunomodulatory agents, steroids, non-steroidal anti-inflammatory agents, antihistamines, analgesics and suitable mixtures thereof.

Additionally, the invention relates to an article of manufacture, which comprises packaging material and a pharmaceutical agent contained within said packaging material, wherein the pharmaceutical agent is therapeutically effective against the medical conditions as described herein, and wherein the packaging material comprises a label or package insert which indicates that the pharmaceutical agent is useful for preventing or treating said medical conditions, and wherein said pharmaceutical agent comprises one or more compounds of Formula (I) according to the invention. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the compounds of the invention (=active compounds) are either employed as such, or particularly in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

Depending upon the particular disease, to be treated or prevented, additional therapeutic active agents, which are normally administered to treat or prevent that disease, may optionally be co-administered with the compounds according to the present invention. As used herein, additional therapeutic agents that are normally administered to treat or prevent a particular disease are known as appropriate for the disease being treated.

In a further aspect of the present invention, the compounds according to this invention or the salts or solvates of said compounds of Formula (I) may be combined with standard therapeutic agents which are commonly used for the treatment of the medical conditions as described herein.

The person skilled in the art is aware on the base of his/her expert knowledge of the total daily dosage(s) and administration form(s) of the additional therapeutic agent(s) co-administered. Said total daily dosage(s) can vary within a wide range. In practicing the present invention and depending on the details, characteristics or purposes of their uses mentioned above, the compounds according to the present invention may be administered in combination therapy separately, sequentially, simultaneously or chronologically staggered (e.g. as combined unit dosage forms, as separate unit dosage forms or a adjacent discrete unit dosage forms, as fixed or nonfixed combinations, as kit-of-parts or as admixtures) with one or more standard therapeutics, in particular art-known chemotherapeutic or target specific anti-cancer agents, such as those mentioned above.

Thus, a further aspect of the present invention is a combination or pharmaceutical composition comprising a first active ingredient, which is a compound according to this invention or a pharmaceutically acceptable salt or solvate thereof, a second active ingredient, which is an art-known standard therapeutic for the medical conditions as described herein, and optionally a pharmacologically acceptable carrier, diluent and/or excipient for sequential, separate, simultaneous or chronologically staggered use in therapy in any order, e.g. to treat, prevent or ameliorate in a patient the medical conditions as described herein. In this context, the present invention further relates to a combination comprising a first active ingredient, which is at least one compound according to this invention, and a second active ingredient, which is at least one art-known standard therapeutic for the medical conditions as described herein, for separate, sequential, simultaneous or chronologically staggered use in therapy, such as e.g. in therapy of those diseases mentioned herein.

The term “combination” according to this invention may be present as a fixed combination, a non-fixed combination or a kit-of-parts. A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously or chronologically staggered.

The first and second active ingredient of a combination or kit-of-parts according to this invention may be provided as separate formulations (i.e. independently of one another), which are subsequently brought together for simultaneous, sequential, separate or chronologically staggered use in combination therapy; or packaged and presented together as separate components of a combination pack for simultaneous, sequential, separate or chronologically staggered use in combination therapy. The type of pharmaceutical formulation of the first and second active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration. The amounts of the first and second active ingredients of the combinations, compositions or kits according to this invention may together comprise a therapeutically effective amount for the treatment, prophylaxis or amelioration of a medical condition as described herein

A further aspect of the present invention is a method for treating co-therapeutically the medical conditions as described herein, in a patient in need of such treatment comprising administering separately, sequentially, simultaneously, fixed or non-fixed a therapeutically effective and tolerable amount of one or more of the compounds according to the present invention and a therapeutically effective and tolerable amount of one or more art-known therapeutic agents for the medical conditions as described herein, to said patient.

References and claims to the use of a compound of the Formula (I) or a pharmaceutically acceptable salt or solvate thereof in the manufacture of a medicament for the treatment of a disease or medical condition in their general and specific forms likewise refer to the corresponding methods of treating said disease or medical condition, said method comprising administering a therapeutically effective and tolerable amount of a compound of the Formula (I) or a pharmaceutically acceptable salt or solvate thereof to a subject in need thereof, compositions comprising a compound of the Formula (I) or a pharmaceutically acceptable salt or solvate thereof for the treatment of said disease or medical condition, a compound of the Formula (I) or a pharmaceutically acceptable salt or solvate thereof for use in the treatment of said disease or medical condition, and vice versa.

For the production of the pharmaceutical compositions, the compounds of the invention (=active compounds) are particularly mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions. The pharmaceutical compositions according to the invention are prepared by processes known per se.

The dosage of the active compounds is carried out in the customary order of magnitude. Topical application forms (such as ointments) thus contain the active compounds in a concentration of, for example, 0.1 to 99%. The customary dose in the case of systemic therapy (p.o.) is usually between 0.3 and 30 mg/kg per day, (i.v.) is usually between 0.3 and 30 mg kg/h. The choice of the optimal dosage regime and duration of medication, particularly the optimal dose and manner of administration of the active compounds necessary in each case can be determined by a person skilled in the art on the basis of his/her expert knowledge.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers.

Furthermore, the compounds of the present invention are partly subject to tautomerism. For example, if a heteroaromatic group containing a nitrogen atom in the ring is substituted with a hydroxy group on the carbon atom adjacent to the nitrogen atom, the following tautomerism can appear:

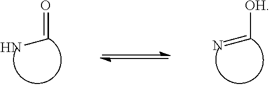

The term “diastereomer” means stereoisomers that are not mirror images of one another and are non-superimposable on one another. The term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

The term “pharmaceutically acceptable salts” refers to salts prepared from pharmaceutically acceptable non-toxic bases, including inorganic bases and organic bases. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The respective salts can be obtained by customary methods which are known to the person skilled in the art like, for example, by contacting these with an organic or inorganic base in a solvent or dispersant, or by cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

The term “solvate” refers to a crystalline form of a molecule that further comprises molecules of a solvent or solvents incorporated into the crystalline lattice structure. Thus, the compounds of the present disclosure may be present in the form of solvates, such as those which include as solvate water, or pharmaceutically acceptable solvates, such as alcohols, in particular ethanol. A stoichiometric or non-stoichiometric amount of solvent is bound by non-covalent intermolecular forces. When the solvent is water, the “solvate” is a “hydrate.” It is understood, that a “pharmaceutically acceptable salts” can in addition optionally contain a “solvate”.

The term “polymorph” as used herein refers to a crystalline form of a compound or a salt, hydrate, or solvate thereof, in a particular crystal packing arrangement. All polymorphs have the same elemental composition. The term “crystalline” as used herein, refers to a solid state form which consists of orderly arrangement of structural units. Different crystalline forms of the same compound, or a salt, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability and solubility.

The term “effective amount” is meant to include the amount of a compound that, when administered, is sufficient to prevent development of, or alleviate to some extent, one or more of the symptoms of a disorder, disease, or condition being treated. The term “effective amount” also refers to the amount of a compound that is sufficient to elicit the biological or medical response of a cell, tissue, system, animal, or human, which is being sought by a researcher, veterinarian, medical doctor, or clinician.

As used herein, the term “subject” refers to any member of the animal kingdom including humans. In some embodiments, “subject” refers to humans, at any stage of development. In some embodiments, “subject” refers to a human patient. In some embodiments, “subject” refers to non-human animals. In some embodiments, the non-human animal is a mammal (e.g. a rodent, a mouse, a rat, a rabbit, a monkey, a dog, a cat, a sheep, cattle, a primate or a pig). In some embodiments, subjects include, but are not limited to, mammals, birds, reptiles, amphibians, fish or worms. In some embodiments, a subject may be a transgenic animal, genetically-engineered animal or a clone.

It has unexpectedly been found that deuterated compounds as detailed herein show higher microsomal stability. The following example section shows further details.

### Experimental Part

The undeuterated matched pairs of the present invention can be prepared as outlined in WO2012/101261, WO2012/101263 and WO2019/048541. The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Abbreviations

Ac acetyl
Boc tert-butyloxycarbonyl
DIPEA N,N-diisopropylethylamine
DMSO dimethylsulfoxide
EA ethyl acetate
ESI electrospray ionization
FCC flash chromatography on silica gel
LCMS liquid chromatography-mass spectrometry
Ms mesylate
PE petroleum ether
rt room temperature (20±4° C.)
TFA trifuoroacetic acid
THF tetrahydrofuran
TMS trimethylsilyl
Ts tosyl

### Experimental Section

### Preparative Example P1

### Step 1: 3-(2-Chloro-6-fluorophenyl)-5-methylisoxazole-4-carbothioamide (P1a)

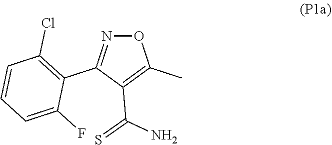 [SMILES: Cc1onc(-c2c(F)cccc2Cl)c1C(N)=S]

A solution of 3-(2-chloro-6-fluorophenyl)-5-methylisoxazole-4-carboxamide (0.80 g, 3.1 mmol) in dry 1,4 dioxane (15 mL), was added Lawesson's reagent (1.3 g). The mixture was stirred at 110° C. for 8 h, cooled to rt, diluted with water and extracted with EA (3×50 mL). The combined organic layer was washed with brine, dried over MgSO₄, concentrated and purified by FCC (PE:EA=5:1) to give compound P1a as a yellow solid. LCMS (ESI): m/z 271.1 (M+H)⁺.

### Step 2: 3-(2-Chloro-6-fluorophenyl)-5-methyl-4-(thiazol-2-yl-d₂)isoxazole (P1b)

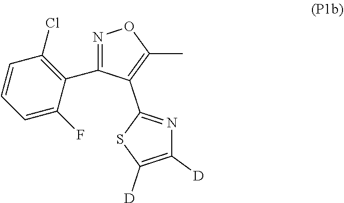 [SMILES: [2H]c1nc(-c2c(-c3c(F)cccc3Cl)noc2C)sc1[2H]]

A solution of compound P1a (0.70 g, 2.6 mmol) in EtOH (10 mL) was added 2-bromoacetaldehyde-d₃ (0.65 g, 5.2 mmol), The mixture was stirred at 80° C. for 24 h, concentrated, diluted with water and extracted with EA (3×50 mL). The combined organic layer was washed with brine, dried over MgSO₄, concentrated and purified by FCC (PE:EA=5:1) to give compound P1b as a yellow solid. LCMS (ESI): m/z 297.0 (M+H)⁺.

### Step 3: (E)-2-(3-(2-Chloro-6-fluorophenyl)-4-(thiazol-2-yl-d₂)isoxazol-5-yl)-N,N-dimethylethen-1-amine (P1c)

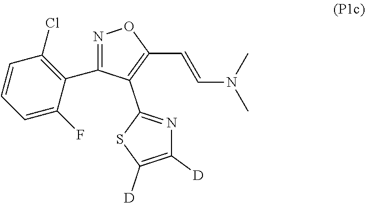 [SMILES: [2H]c1nc(-c2c(-c3c(F)cccc3Cl)noc2/C=C/N(C)C)sc1[2H]]

A solution of compound P1b (0.50 g, 1.7 mmol) in dry toluene (10 mL) was added 1-tert-butoxy-N,N,N′,N′-tetramethylmethanediamine (0.74 g). The mixture was stirred at 110° C. for 16 h, cooled to rt, diluted with water and extracted with EA (3×50 mL). The combined organic layer was washed with brine, dried over MgSO₄, concentrated and purified by FCC (PE:EA=10:1) to give compound P1c as a yellow solid. LCMS (ESI): m/z 352.1 (M+H)⁺.

### Step 4: (E)-2-(3-(2-Chloro-6-fluorophenyl)-4-(thiazol-2-yl-d₂)isoxazol-5-yl)-N,N-dimethylethen-1-amine (P1)

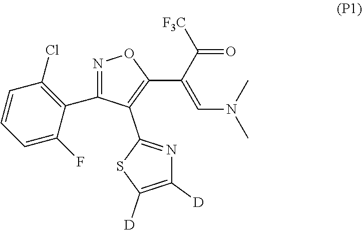 [SMILES: [2H]c1nc(-c2c(-c3c(F)cccc3Cl)noc2/C(=C/N(C)C)C(=O)C(F)(F)F)sc1[2H]]

A solution of compound P1c (0.50 g, 1.4 mmol) in dry CH₂Cl₂ (10 mL), was slowly added trifluoroacetic anhydride (0.25 mL) at 0° C., The mixture was stirred at 0° C. for 6 h, quenched with water and extracted with EA (3×50 mL). The combined organic layer was washed with brine, dried over MgSO₄ and concentrated to give compound P1 as a yellow solid, which was used in the next step without further purification.

### Preparative Example P2

### Step 1: Pyrimidin-d₃-2-amine (P2a)

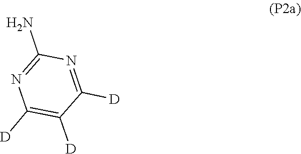

A mixture of pyrimidin-2-amine (10.0 g, 105 mmol) and 10% palladium on carbon (1.0 g) in deuterium oxide (50 mL) was stirred at about 110° C. in a sealed tube under a hydrogen atmosphere for about 24 h. After cooling, the mixture is diluted with methanol (10 mL) and filtered to remove the catalyst. The filtered catalyst is washed with methanol (2×10 mL) and the filtrate is concentrated and purified by FCC (PE:EA=2:1) to give compound P2a. LCMS (ESI): m/z 99.2 (M+H)⁺.

### Step 2: 2-Chloropyrimidine-4,5,6-d₃ (P2b)

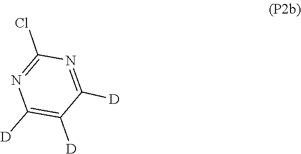

To a solution of compound P2a (7.5 g, 76 mmol) in water (160 mL) was added HCl (40 mL) and ZnCl₂ (36 g) at rt. the mixture was stirred at rt for 30 min. Then NaNO₂ (13 g in 20 mL H₂O) was added dropwise at −5° C. and the mixture was stirred for 1 h and extracted with CH₂Cl₂ (3×30 mL). The organic layer was concentrated and purified by FCC (PE:EA=5:1) to afford compound P2b as white solid.

LCMS (ESI): m/z 118.1 (M+H)⁺.

### Step 3: 2-Methylpyrimidine-4,5,6-d₃ (P2)

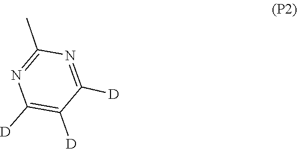

To a solution of compound P2b (2.2 g, 19 mmol) in THF (40 mL) was added K₂CO₃ (5.2 g), trimethylboroxin (3.5M in THF, 8.0 mL) and Pd(dppf)Cl₁₂ (278 mg). The mixture was stirred at 80° C. for 4 h under N₂, cooled to rt, diluted with water and extracted with CH₂Cl₂. The organic layer was washed with water, brine and dried over MgSO₄, concentrated and purified by FCC (CH₂Cl₂) to furnish compound P2 as a colorless oil. LCMS (ESI): m/z 98.2 (M+H)⁺.

### Preparative Example P3

### Step 1: 2,6-Dichlorobenzaldehyde-3,4,5-d₃ (P3a)

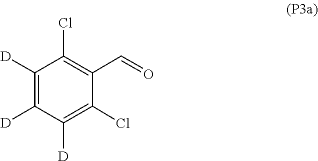 [SMILES: [2H]c1c([2H])c(Cl)c(C=O)c(Cl)c1[2H]]

To a solution of benzaldehyde-2,3,4,5,6-d₅ (5.0 g, 45 mmol), NCS (15 g), 2-amino-6-nitrobenzoic acid (8.2 g), silver trifluoroacetate (1.0 g) in 1,2-dichloroethane (250 mL) was added Pd(OAc)₂ (0.50 g) and TFA (50 g) under N₂. The mixture was stirred at 60° C. for 5 days, cooled to rt, diluted with water (100 mL) and adjusted to pH=7 with aq. NaHCO₃ and the solid was filtered off. The aqueous solution was extracted with CH₂Cl₂ (3×100 mL). The combined organic layer was washed with brine (100 mL), dried over Na₂SO₄, concentrated and purified by FCC (PE:EA=25:1) to afford compound P3a as white solid.

### Step 2: 2-Chloro-6-fluorobenzaldehyde-3,4,5-d₃ (P3b)

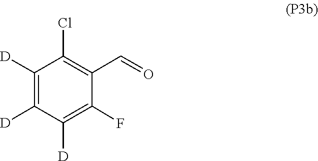 [SMILES: [2H]c1c([2H])c(F)c(C=O)c(Cl)c1[2H]]

To a solution of compound P3a (6.0 g, 34 mmol) in DMSO (50 mL) was added KF (2.4 g). The mixture was stirred at 160° C. under N₂ for 4 h, cooled to rt, diluted with water (100 mL) and extracted with CH₂Cl₂ (3×100 mL). The combined organic layer was washed with brine (100 mL), dried over Na₂SO₄, concentrated and purified by FCC (PE:EA=25:1) to afford compound P3b as white solid.

### Step 3: (E)-2-Chloro-6-fluorobenzaldehyde-3,4,5-d₃ oxime (P3c)

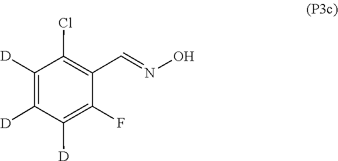 [SMILES: [2H]c1c([2H])c(F)c(/C=N/O)c(Cl)c1[2H]]

To a solution of compound P3b (1.1 g, 6.8 mmol) in THF (10 mL) was added hydroxylamine hydrate (0.51 g) and the mixture was stirred at 60° C. for 1 h, concentrated and purified by FCC (A: water (10 mmol/L NH₄CO₃) B: MeCN) to give compound P3c as white solid. LCMS (ESI): m/z 177.0 (M+H)⁺.

### Step 4: (Z)-2-Chloro-6-fluoro-N-hydroxybenzimidoyl chloride-3,4,5-d₃ (P3)

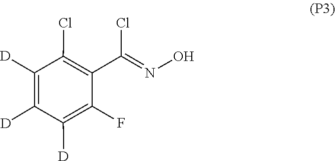 [SMILES: [2H]c1c([2H])c(F)c(/C(Cl)=N/O)c(Cl)c1[2H]]

To a solution of compound P3c (0.50 g, 2.8 mmol) ) in DMF (5 mL) were slowly added NCS (0.38 g) at 0° C. The mixture was stirred at rt for 1 h, diluted with water (30 mL) and extracted with EA (3×30 mL). The combined organic layer was washed with brine (20 mL), dried over Na₂SO₄ and concentrated in vacuum to afford compound P3 as colorless oil, which was used in the next step without further purification.

### Preparative Example P4: (3-Chlorophenyl-2,4,6-d₃)hydrazine (P4)

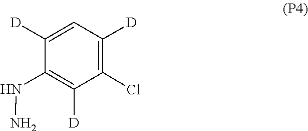 [SMILES: [2H]c1cc([2H])c(NN)c([2H])c1Cl]

To a mixture of 3-chlorobenzen-2,4,6-d₃-amine (0.40 g, 3.1 mmol) in 36% aqueous HCl (20 mL) was slowly added sodium nitrite (0.25 g) at −10° C., the resulting mixture was stirred at −10° C. for 1 h. Then tin dichloride dehydrate (0.87 g) in 36% aqueous HCl (10 mL) was added. The resulting mixture was stirred at −5° C. for 2 h and warmed to room temperature for 12 h, quenched with saturated aq. NaOH to pH 7-8 and extracted with EA (3×20 mL). The combined organic layer was washed with brine (50 mL), dried over Na₂SO₄ and purified by FCC (PE:EA=5:1) to give compound P4 as a colorless oil.

LCMS (ESI): m/z 146.1 (M+H)⁺.

### Example 1

### Step 1: (S)-Propane-1,1-d₂-1,2-diol (1a)

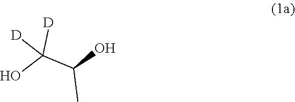

To a solution of methyl (S)-2-hydroxypropanoate (5.0 g, 4.8 mmol) in dry THF (30 mL) was added LiAlD₄ (242 mg, 5.76 mmol) at 0° C. and the mixture was stirred for 2 h at this temperature. The mixture was quenched by water (0.25 mL). Then 10% NaOH (0.5 mL) and water (0.75 mL) were added. The mixture was filtered and concentrated to afford compound 1a as a colorless oil, which was used for the next step without purification. LCMS (ESI): m/z 101.1 (M+Na)⁺.

### Step 2: (S)-2-Hydroxypropyl-1,1-d₂₋₄-methylbenzenesulfonate (1b)

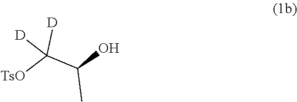 [SMILES: [2H]C([2H])(OS(=O)(=O)c1ccc(C)cc1)[C@H](C)O]

To a solution of compound 1a (3.6 g, 4.6 mmol) and Et₃N (1.93 mL, 14 mmol) in DCM (30 mL) was added p-toluenesulfonyl chloride (968 mg, 5.08 mmol). The mixture was stirred at rt overnight. The resulting suspension was filtered and the filter liquor was concentrated under vacuum. The residue was purified by FCC (PE:EA=2:1) to give compound 1b as a colorless oil.

### Step 3: (S)-1-Hydrazineylpropan-1,1-d₂₋₂-ol (1c)

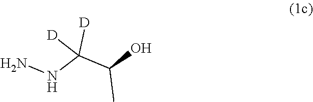

To a solution of compound 1b (1.0 g, 4.30 mmol) in EtOH (20 mL) was added hydrazine monohydrate (0.4 mL) and the mixture was heated to 60° C. for 2 h and concentrated under vacuum to afford compound 1c as a yellow oil, which was used for the next step without purification. LCMS (ESI): m/z 93.2 (M+H)⁺.

### Step 4: Ethyl (S)-1-(2-hydroxypropyl-1,1-d₂)-5-(trifluoromethyl)-1H-pyrazole-4-carboxylate (1d)

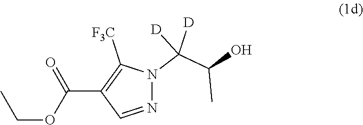 [SMILES: [2H]C([2H])([C@H](C)O)n1ncc(C(=O)OCC)c1C(F)(F)F]

To a solution of compound 1c (1.2 g, crude) and ethyl 2-((dimethylamino)methylene)-4,4,4-trifluoro-3-oxobutanoate (1.5 g, 6.3 mmol) in EtOH (20 mL) was added Et₃N (1.8 mL, 13 mmol). The solution was stirred at rt for 16 h, diluted with water and extracted with EA (3×20 mL) and washed with brine (20 mL). The combined organic layer was dried over Na₂SO₄, concentrated and purified by FCC (PE:EA=5:1) to give compound 1d as a colorless oil. LCMS (ESI): m/z 269.3 (M+H)⁺.

### Step 5: Ethyl (S)-5-(trifluoromethyl)-1-(2-((trimethylsilyl)oxy)propyl-1,1-d₂)-1H-pyrazole-4-carboxylate (1e)

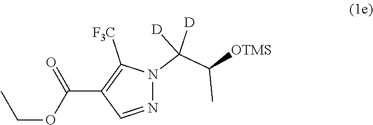

A solution of compound 1d (490 mg, crude) in (Me₃Si)₂NH (20 mL) was added TMSCl (0.35 mL). The mixture was stirred at rt for 16 h, diluted with DCM (30 mL) and washed with water (20 mL) and saturated aq. NH₄Cl₁ (20 mL). The organic layer was dried over Na₂SO₄, concentrated and purified by FCC (PE:EA=10:1) to give compound 1e as a colorless oil. LCMS (ESI): m/z 341.2 (M+H)⁺.

### Step 6: (S)-2-(Pyrimidin-2-yl)-1-(5-(trifluoromethyl)-1-(2-((trimethylsilyl)oxy)propyl-1,1-d₂)-1H-pyrazol-4-yl)ethan-1-one (1f)

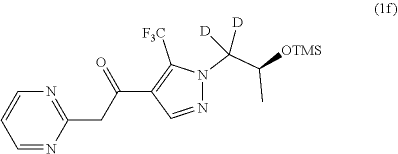

To a solution of compound 1e (250 mg, 0.73 mmol) in THF (10 mL) was added LiHMDS (1.6M in THF, 1.0 mL) at −10° C. under a N₂ atmosphere. The mixture was stirred at this temperature for 30 min and then 2-methylpyrimidine (83 mg, 0.88 mmol) was added. The mixture was stirred at rt for 8 h, quenched with water (20 mL) and extracted with EA (3×20 mL). The combined organic layer was dried over Na₂SO₄, concentrated and purified by FCC (PE:EA=1:1) to give compound if as a yellow solid.

LCMS (ESI): m/z 389.1 (M+H)⁺.

### Step 7: (S)-1-(4-(3-(2-Chloro-6-fluorophenyl)-4-(pyrimidin-2-yl)isoxazol-5-yl)-5-(trifluoromethyl)-1H-pyrazol-1-yl)propan-1,1-d₂₋₂-ol (1)

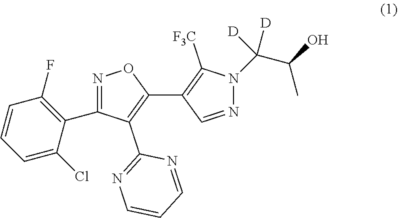 [SMILES: [2H]C([2H])([C@H](C)O)n1ncc(-c2onc(-c3c(F)cccc3Cl)c2-c2ncccn2)c1C(F)(F)F]

To a solution of compound if (140 mg, 0.36 mmol) in dry DMF (2 mL) was added NaH (72 mg, 60% wt, 1.80 mmol) at 0° C. The mixture was allowed to reach rt for 10 min, then (Z)-2-chloro-6-fluoro-N-hydroxybenzimidoyl chloride (74 mg, 0.36 mmol) was added and the mixture was stirred at 60° C. for 3 h, cooled to rt, quenched with saturated aq. NH₄Cl solution and extracted with EA (3×20 mL). The combined organic layer was dried over Na₂SO₄ and concentrated under reduced pressure. The residue was dissolved in MeOH (2 mL) and HCl in MeOH (3M, 0.1 mL) was added. The mixture was stirred at rt for 30 min, quenched with water (10 mL) and saturated aq. NaHCO₃ (0.2 mL) and then extracted with EA (3×10 mL). The combined organic was dried over Na₂SO₄, concentrated and purified by reversed-phase flash chromatography (C18) (0.1% NH₄HCO₃ in water, 10% to 100% MeCN) to give target compound 1 as a white solid. ¹H-NMR (500 MHz, DMSO-d₆) δ 8.61 (d, J=4.5 Hz, 2H), 8.22 (s, 1H), 7.65-7.59 (m, 1H), 7.48 (d, J=8.5 Hz, 1H), 7.41 (t, J=8.8 Hz, 1H), 7.33 (t, J=4.8 Hz, 1H), 5.10 (d, J=5.5 Hz, 1H), 4.16-4.13 (m, 1H), 1.13 (d, J=6.0 Hz, 3H). LCMS (ESI): m/z 470.1 (M+H)⁺.

### Example 1/1

### 1-(4-(3-(2-Chloro-6-fluorophenyl)-4-(pyrimidin-2-yl)isoxazol-5-yl)-5-(trifluoromethyl)-1H-pyrazol-1-yl)propan-1,1,2-d₃₋₂-ol (1/1)

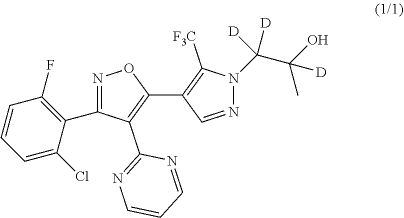 [SMILES: [2H]C(C)(O)C([2H])([2H])n1ncc(-c2onc(-c3c(F)cccc3Cl)c2-c2ncccn2)c1C(F)(F)F]

The Example 1/1 was prepared as described in Example 1 by using the methyl 2-oxopropanoate as starting material. ¹H-NMR (400 MHz, DMSO-d₆) δ 8.61 (d, J=4.8 Hz, 2H), 8.23 (s, 1H), 7.65-7.59 (m, 1H), 7.48 (d, J=8.4 Hz, 1H), 7.41 (t, J=8.6 Hz, 1H), 7.34 (t, J=5.0 Hz, 1H), 5.09 (s, 1H), 1.12 (s, 3H). LCMS (ESI): m/z 471.0 (M+H)⁺.

### Comparative Example C1/1 and C1/2

The following Comparative Examples were prepared similar as described above by using the appropriate building blocks (at the respective stage).

| # | building block | structure | analytical data |
|---|---|---|---|
| C1/1 | 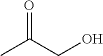 | 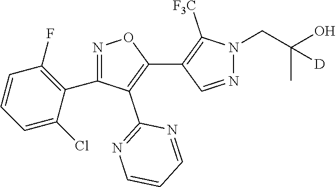 [SMILES: [2H]C(C)(O)Cn1ncc(-c2onc(-c3c(F)cccc3Cl)c2-c2ncccn2)c1C(F)(F)F] | ¹H-NMR (500 MHz, CD₃OD) δ 8.52 (d, J = 4.5 Hz, 2H), 8.03 (s, 1H), 7.54-7.49 (m, 1H), 7.35 (d, J = 8.5 Hz, 1H), 7.23-7.19 (m, 2H), 4.30 (s, 2H), 1.24 (s, 3H). LCMS (ESI): m/z 469.1 (M + H)⁺ |
| C1/2 | 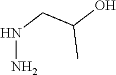 | 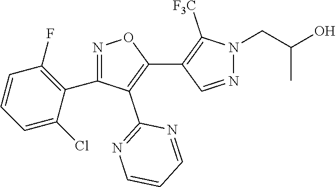 [SMILES: CC(O)Cn1ncc(-c2onc(-c3c(F)cccc3Cl)c2-c2ncccn2)c1C(F)(F)F] | ¹H-NMR (500 MHz, CD₃OD) δ 8.52 (d, J = 5.0 Hz, 2H), 8.03 (s, 1H), 7.54-7.49 (m, 1H), 7.35 (d, J = 8.0 Hz, 1H), 7.23-7.19 (m, 2H), 4.36-4.29 (m, 3H), 1.25 (d, J = 5.5 Hz, 3H). LCMS (ESI): m/z 468.1 (M + H)⁺ |

### Example 2

### Step 1: tert-Butyl 2-(3-hydroxy-3-methylcyclobutyl-1-d)hydrazine-1-carboxylate (2a)

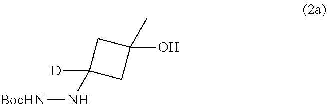 [SMILES: [2H]C1(NNC(=O)OC(C)(C)C)CC(C)(O)C1]

To a solution of 3-hydroxy-3-methylcyclobutan-1-one (0.56 g, 5.6 mmol) in MeOD (5 mL) was added BocNHNH₂ (896 mg, 6.78 mmol) and AcOD (2 drops) at rt. The mixture was stirred at rt for 2 h and 80° C. overnight, evaporated, diluted with water (30 mL) and extracted with EA (3×30 mL). The combined organic layer was washed with brine (30 mL), dried over Na₂SO₄, filtered and concentrated to afford compound 2a as a sticky oil. LCMS (ESI): m/z 240.1 (M+Na)⁺.

### Step 2: 3-Hydrazineyl-1-methylcyclobutan-3-d-1-ol hydrochloride (2b)

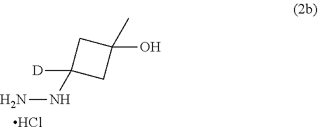

To a solution of compound 2a (600 mg) in MeOH (1 mL) was added HCl in MeOH (3M, 5 mL) at rt.

The mixture was stirred at rt for 2 h and concentrated to afford compound 2b as a yellow solid. LCMS (ESI): m/z 118.1 (M-C₁)⁺.

### Step 3: Ethyl 1-(3-hydroxy-3-methylcyclobutyl-1-d)-5-(trifluoromethyl)-1H-pyrazole-4-carboxylate (2c)

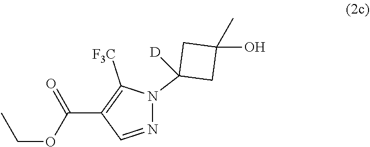 [SMILES: [2H]C1(n2ncc(C(=O)OCC)c2C(F)(F)F)CC(C)(O)C1]

To a solution of compound 2b (300 mg, crude) and ethyl 2-((dimethylamino)methylene)-4,4,4-trifluoro-3-oxobutanoate (760 mg, 3.18 mmol) in EtOH (10 mL) was added DIPEA (992 mg, 7.68 mmol). The solution was stirred at rt for 16 h, diluted with water and extracted with EA (3×30 mL). The combined organic layer was washed with brine (20 mL), dried over Na₂SO₄, concentrated and purified by FCC (PE:EA=5:1) to give compound 2c as a colorless oil. LCMS (ESI): m/z 294.0 (M+H)⁺.

### Step 4: Ethyl 1-(3-methyl-3-((trimethylsilyl)oxy)cyclobutyl-1-d)-5-(trifluoromethyl)-1H-pyrazole-4-carboxylate (2d)

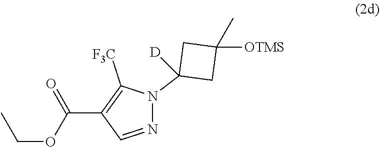

A solution of compound 2c was treated as described in Example 1, step 5 to give compound 2d as a colorless oil. LCMS (ESI): m/z 366.2 (M+H)⁺.

### Step 5: 1-(1-(3-Methyl-3-((trimethylsilyl)oxy)cyclobutyl-1-d)-5-(trifluoromethyl)-1H-pyrazol-4-yl)-2-(pyrimidin-2-yl)ethan-1-one (2e)

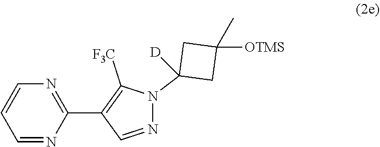

To a solution of compound 2d was treated as described in Example 1, step 6 to give compound 2e as a yellow solid. (ESI): m/z 414.2 (M+H)⁺.

### Step 6: (1r,3r)-3-(4-(3-(2-Chloro-6-fluorophenyl)-4-(pyrimidin-2-yl)isoxazol-5-yl)-5-(trifluoro-methyl)-1H-pyrazol-1-yl)-1-methylcyclobutan-3-d-1-ol (2-1) and (1s,3s)-3-(4-(3-(2-chloro-6-fluorophenyl)-4-(pyrimidin-2-yl)isoxazol-5-yl)-5-(trifluoromethyl)-1H-pyrazol-1-yl)-1-methylcyclobutan-3-d-1-ol (2-2)

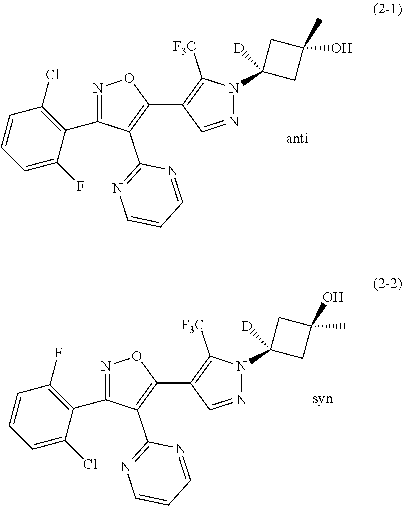 [SMILES: [2H][C@]1(n2ncc(-c3onc(-c4c(F)cccc4Cl)c3-c3ncccn3)c2C(F)(F)F)C[C@](C)(O)C1 ; [2H][C@]1(n2ncc(-c3onc(-c4c(F)cccc4Cl)c3-c3ncccn3)c2C(F)(F)F)C[C@@](C)(O)C1]

To a solution of compound 2e (60 mg, 0.15 mmol) in dry THF (2 mL) was added NaH (15 mg, 60% wt, 0.39 mmol) at 0° C. The mixture was allowed to reach rt for 10 min, then (Z)-2-chloro-6-fluoro-N-hydroxybenzimidoyl chloride (47 mg, 0.23 mmol) was added and the mixture was stirred at rt for overnight. The mixture was quenched with a saturated aq. NH₄C₁ solution and extracted with EA (3×20 mL). The combined organic layer was dried over Na₂SO₄, concentrated and dissolved in MeOH (2 mL). A solution of HCl in MeOH (3M, 0.1 mL) was added and the mixture was stirred at rt for 30 min, quenched with water (10 mL) and saturated aq. NaHCO₃ solution (0.2 mL) and then extracted with EA (3×10 mL). The combined organic layer was concentrated and purified by prep-TLC (MeOH:DCM=20:1) and then combi-flash (0.1% NH₄HCO₃ in water, 10% to 100% MeCN) to give separated compound 2-1 and compound 2-2 as off-white solids, respectively. Compound 2-1: ¹H-NMR (400 MHz, CDCl₃) δ 8.49 (d, J=4.8 Hz, 2H), 8.02 (s, 1H), 7.39-7.35 (m, 1H), 7.28-7.26 (m, 1H), 7.13-7.03 (m, 2H), 2.87-2.83 (m, 2H), 2.66-2.61 (m, 2H), 1.62 (s, 1H), 1.56 (d, J=3.2 Hz, 3H). LCMS (ESI): m/z 495.1 (M+H)⁺. Compound 2-2: ¹H-NMR (400 MHz, CDCl₃) δ 8.49 (d, J=4.8 Hz, 2H), 8.06 (s, 1H), 7.42-7.36 (m, 1H), 7.28-7.26 (m, 1H), 7.13-7.04 (m, 2H), 2.96 (br s, 1H), 2.87-2.75 (m, 4H), 1.49 (s, 3H). LCMS (ESI): m/z 495.1 (M+H)⁺.

### Example 2-1/1 to 2-2/1

The following Examples were prepared as described in Example 2 by using the appropriate building block in step 6:

| | building | | |
|---|---|---|---|
| # | block | structure | analytical data |
| 2-1/1 | 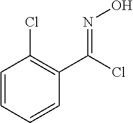 [SMILES: O/N=C(\Cl)c1ccccc1Cl] | 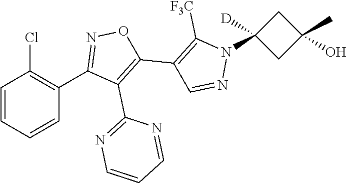 [SMILES: [2H][C@]1(n2ncc(-c3onc(-c4ccccc4Cl)c3-c3ncccn3)c2C(F)(F)F)C[C@](C)(O)C1] | ¹H-NMR (400 MHz, CDCl₃) δ 8.53 (d, J = 5.2 Hz, 2H), 8.05 (s, 1H), 7.65-7.62 (m, 1H), 7.43- 7.37 (m, 3H), 7.08 (t, J = 5.0 Hz, 1H), 2.86- 2.82 (m, 2H), 2.64-2.60 (m, 2H), 1.62 (s, 1H), 1.56 (s, 3H). LCMS (ESI): m/z 477.1 (M + H)⁺ |
| 2-2/1 | | 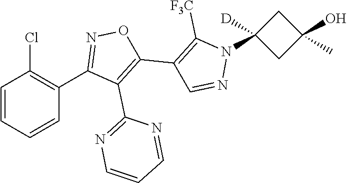 [SMILES: [2H][C@]1(n2ncc(-c3onc(-c4ccccc4Cl)c3-c3ncccn3)c2C(F)(F)F)C[C@@](C)(O)C1] | ¹H-NMR (400 MHz, CDCl₃) δ 8.54 (d, J = 5.2 Hz, 2H), 8.09 (s, 1H), 7.65-7.62 (m, 1H), 7.43- 7.36 (m, 3H), 7.09 (t, J = 4.8 Hz, 1H), 2.86- 2.74 (m, 4H), 1.48 (s, 3H). LCMS (ESI): m/z 477.1 (M + H)⁺ |

### Example 3

### Step 1: 3-Methylbutane-1,1-d₂-1,3-diol (3a)

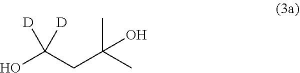

To a solution of ethyl 3-hydroxy-3-methylbutanoate (5.0 g, 34 mmol) in dry THF (25 mL) was added LiAlD₄ (1.72 g, 410 mmol) at 0° C. and the mixture was stirred for 2 h at this temperature. The mixture was quenched by water (1.72 mL). Then 10% NaOH (3.44 mL) and water (5.16 mL) were added. The mixture was filtered and concentrated to afford compound 3a as a colorless oil, which was used for the next step without further purification.

### Step 2: 3-Hydroxy-3-methylbutyl-1,1-d₂₋₄-methylbenzenesulfonate (3b)

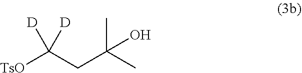 [SMILES: [2H]C([2H])(CC(C)(C)O)OS(=O)(=O)c1ccc(C)cc1]

To a solution of compound 3a (3.2 g, 30.1 mmol) and Et₃N (12.6 mL, 90.6 mmol) in DCM (50 mL) were added p-toluenesulfonyl chloride (6.90 g, 36.2 mmol). The mixture was stirred at rt overnight. The resulting suspension was filtered and the filter liquor was concentrated under vacuum. The residue was purified by FCC (PE:EA=2:1) to give compound 3b as a colorless oil.

### Step 3: 4-Hydrazineyl-2-methylbutan-4,4-d₂₋₂-ol (3c)

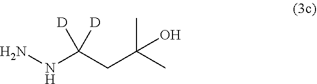

To a solution of compound 3b (3.3 g, 12.7 mmol) in EtOH (40 mL) was added hydrazine monohydrate (15 mL) and the mixture was heated to 60° C. for 2 h then concentrated to approx. 10 mL volume. Saturated aq. NaOH (30 mL) and THF (60 mL) were added and the organic layer was separated, dried over Na₂SO₄, filtered and concentrated to afford compound 3c as a yellow oil, which was used for the next step without further purification. LCMS (ESI): m/z 121.3 (M+H)⁺.

### Step 4: Ethyl 1-(3-hydroxy-3-methylbutyl-1,1-d₂)-5-(trifluoromethyl)-1H-pyrazole-4-carboxylate (3d)

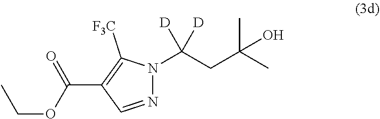 [SMILES: [2H]C([2H])(CC(C)(C)O)n1ncc(C(=O)OCC)c1C(F)(F)F]

To a solution of ethyl 2-((dimethylamino)methylene)-4,4,4-trifluoro-3-oxobutanoate (2.39 g, 10.0 mmol) and compound 3c (0.60 g, 5.0 mmol) in EtOH (25 mL) was added Et₃N (2.1 mL, 15 mmol). The solution was stirred at rt for 16 h, diluted with water and extracted with EA (3×20 mL) and washed with brine (20 mL). The combined organic layer was dried over Na₂SO₄, concentrated and purified by FCC (PE:EA=5:1) to give compound 3d as a colorless oil.

### Step 5: Ethyl 1-(3-methyl-3-((trimethylsilyl)oxy)butyl-1,1-d₂)-5-(trifluoromethyl)-1H-pyrazole-4-carboxylate (3e)

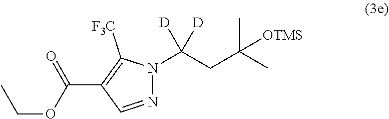

A solution of compound 3d (370 mg, 1.25 mmol) in (Me₃Si)₂NH (10 mL) was added TMSCl (162 mg, 1.5 mmol). The mixture was stirred at rt for 16 h, diluted with DCM (30 mL) and washed with water (20 mL) and saturated aq. NH₄Cl (20 mL). The organic layer was dried over Na₂SO₄, concentrated and purified by FCC (PE:EA=10:1) to give compound 3e as a colorless oil.

### Step 6: 1-(1-(3-Methyl-3-((trimethylsilyl)oxy)butyl-1,1-d₂)-5-(trifluoromethyl)-1H-pyrazol-4-yl)-2-(pyrimidin-2-yl)ethan-1-one (3f)

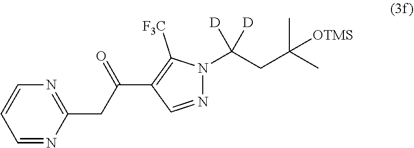

To a solution of compound 3e (260 mg, 706 μmol) in dry THF (8 mL) was added LiHMDS (1.6M in THF, 1 mL) −10° C. under a N₂ atmosphere. The mixture was stirred at this temperature for 30 min and then 2-methylpyrimidine (75 mg, 0.80 mmol) was added. The mixture was stirred at rt for 8 h, quenched with water (20 mL) and extracted with EA (3×35 mL). The combined organic layer was dried over Na₂SO₄, concentrated and purified by FCC (PE:EA=1:1) to give compound 3f as a yellow solid.

### Step 7: 3-(2-Chloro-6-fluorophenyl)-5-(1-(3-methyl-3-((trimethylsilyl)oxy)butyl-1,1-d₂)-5-(trifluoro-methyl)-1H-pyrazol-4-yl)-4-(pyrimidin-2-yl)isoxazole (3g)

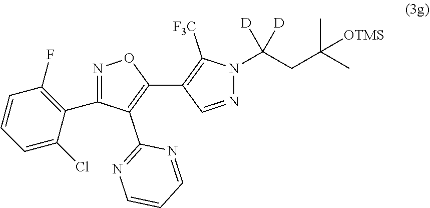

To a solution of compound 3f (200 mg, 0.48 mmol) in dry DMF (10 mL) was added NaH (96 mg, 60% wt, 2.5 mmol) at 0° C. The mixture was allowed to reach rt for 20 min, then (Z)-2-chloro-6-fluoro-N-hydroxybenzimidoyl chloride (149 mg, 716 μmol) was added and the mixture was stirred at 60° C. for 3 h, cooled to rt, quenched with saturated aq. NH₄Cl solution and extracted with EA (3×50 mL). The combined organic layer was dried over Na₂SO₄, concentrated and purified by FCC (PE:EA=5:1) to give compound 3g as a colorless oil.

### Step 8: 4-(4-(3-(2-Chloro-6-fluorophenyl)-4-(pyrimidin-2-yl)isoxazol-5-yl)-5-(trifluoromethyl)-1H-pyrazol-1-yl)-2-methylbutan-4,4-d₂-2-ol (3)

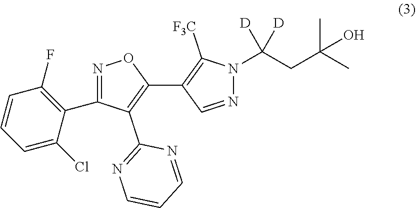 [SMILES: [2H]C([2H])(CC(C)(C)O)n1ncc(-c2onc(-c3c(F)cccc3Cl)c2-c2ncccn2)c1C(F)(F)F]

To a solution of compound 3g (180 mg, 316 μmol) in MeOH (5 mL) was added HCl in MeOH (2M, 1 mL) and the mixture was stirred at rt for 1 h, concentrated and purified by reversed-phase chromatography (C18) (MeCN:0.05% NH₄HCO₃₌₁₀ to 100%) to give compound 3 as a white solid. ¹H-NMR (500 MHz, DMSO-d₆) δ 8.62 (d, J=5.0 Hz, 2H), 8.21 (s, 1H), 7.65-7.60 (m, 1H), 7.48 (d, J=8.5 Hz, 1H), 7.41 (t, J=9.0 Hz, 1H), 7.34 (t, J=4.8 Hz, 1H), 4.58 (s, 1H), 1.97 (s, 2H), 1.18 (s, 6H). LCMS (ESI): m/z 498.3 (M+H)⁺.

### Example 3-1 to 3-3

The following Examples were prepared as described in Example 3 by using the appropriate building block:

| # | building block | structure | analytical data |
|---|---|---|---|
| 3-1 | 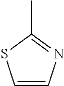 | 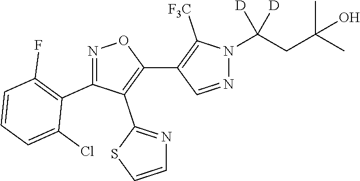 [SMILES: [2H]C([2H])(CC(C)(C)O)n1ncc(-c2onc(-c3c(F)cccc3Cl)c2-c2nccs2)c1C(F)(F)F] | ¹H-NMR (400 MHz, CD₃OD) δ 8.03 (s, 1H), 7.70 (d, J = 3.6 Hz, 1H), 7.65-7.58 (m, 1H), 7.50 (d, J = 3.2 Hz, 1H), 7.43 (d, J = 8.4 Hz, 1H), 7.32-7.27 (m, 1H), 2.11 (s, 2H), 1.29 (s, 6H). LCMS (ESI): m/z 503.1 (M + H)⁺ |
| 3-2 | 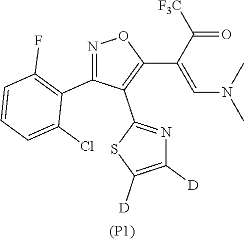 | 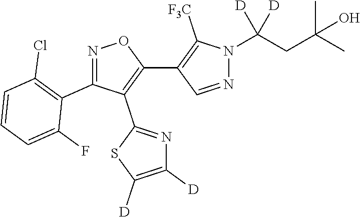 [SMILES: [2H]c1nc(-c2c(-c3c(F)cccc3Cl)noc2-c2cnn(C([2H])([2H])CC(C)(C)O)c2C(F)(F)F)sc1[2H]] | ¹H-NMR (400 MHz, CD₃OD) δ 8.03 (s, 1H), 7.65-7.58 (m, 1H), 7.45 (d, J = 8.4 Hz, 1H), 7.30 (dt, J = 0.7, 8.8 Hz, 1H), 2.10 (s, 2H), 1.29 (s, 6H). LCMS (ESI): m/z 505.0 (M + H)⁺ |
| 3-3 | 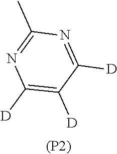 | 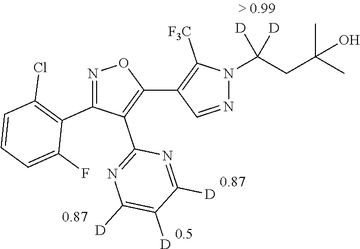 [SMILES: [2H]c1nc(-c2c(-c3c(F)cccc3Cl)noc2-c2cnn(C([2H])([2H])CC(C)(C)O)c2C(F)(F)F)nc([2H])c1[2H]] | According NMR, the deuteration ratio is as depicted in the chemical formula. ¹H- NMR (400 MHz, DMSO-d₆) δ 8.62-8.60 (m, 0.26H), 8.21 (s, 1H), 7.65-7.60 (m, 1H), 7.48 (d, J = 8.0 Hz, 1H), 7.41 (t, J = 8.8 Hz, 1H), 7.34-7.33 (m, 0.48H), 4.58 (s, 1H), 1.97 (s, 2H), 1.18 (s, 6H). LCMS (ESI): m/z 501.0 (M + H)⁺ |

### Example 4

### Step 1: Ethyl 2-((dimethylamino)methylene)-4,4-difluoro-3-oxobutanoate (4a)

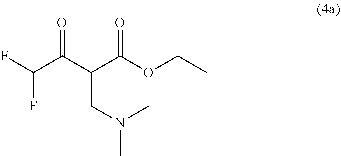

To a solution of ethyl 4,4-difluoro-3-oxobutanoate (10.0 g, 60.2 mmol) in toluene (20 mL) was added 1,1-dimethoxy-N,N-dimethylmethanamine (7.17 g, 60.2 mmol) and the mixture was stirred to rt for 16 h, concentrated under vacuum to afford compound 4a as a yellow oil which, was used for the next step without further purification LCMS (ESI): m/z 222.1 (M+H)⁺.

### Step 2: 4-(4-(3-(2-Chloro-6-fluorophenyl)-4-(pyrimidin-2-yl)isoxazol-5-yl)-5-(difluoromethyl)-1H-pyrazol-1-yl)-2-methylbutan-4,4-d₂₋₂-ol (4)

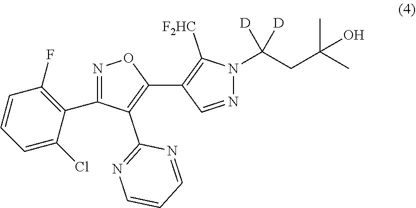 [SMILES: [2H]C([2H])(CC(C)(C)O)n1ncc(-c2onc(-c3c(F)cccc3Cl)c2-c2ncccn2)c1C(F)F]

Compound 4a was treated as described in Example 3, step 4 to step 8 to give compound 4 as a white solid. ¹H-NMR (500 MHz, DMSO-d₆) δ 8.67 (d, J=5.0 Hz, 2H), 8.21 (s, 1H), 7.64-7.58 (m, 1H), 7.54-7.33 (m, 4H), 4.56 (br s, 1H), 1.95 (s, 2H), 1.18 (s, 6H). LCMS (ESI): m/z 480.2 (M+H)⁺.

### Example 4-1/1 to 4-2/1

The following Examples were prepared as described in Example 2 by using the appropriate building block 4a in step 3:

| # | building block | structure | analytical data |
|---|---|---|---|
| 4-1/1 | 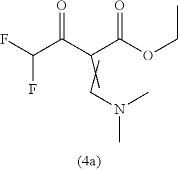 | 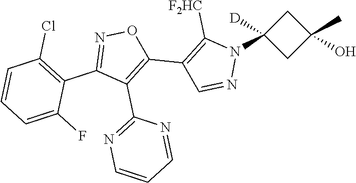 [SMILES: [2H][C@]1(n2ncc(-c3onc(-c4c(F)cccc4Cl)c3-c3ncccn3)c2C(F)F)C[C@](C)(O)C1] | ¹H-NMR (400 MHz, CDCl₃) δ 8.58 (d, J = 4.8 Hz, 2H), 8.31 (s, 1H), 7.42-7.35 (m, 1H), 7.27-7.25 (m, 2H), 7.13-7.07 (m, 2H), 2.84-2.80 (m, 2H), 2.66-2.62 (m, 2H), 1.56 (s, 3H). LCMS (ESI): m/z 477.0 (M + H)⁺ |
| 4-2/1 | | 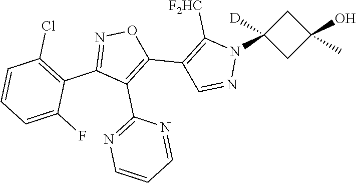 [SMILES: [2H][C@]1(n2ncc(-c3onc(-c4c(F)cccc4Cl)c3-c3ncccn3)c2C(F)F)C[C@@](C)(O)C1] | ¹H-NMR (400 MHz, CDCl₃) δ 8.58 (d, J = 4.8 Hz, 1H), 8.36 (s, 1H), 7.42-7.36 (m, 1H), 7.27-7.25 (m, 2H), 7.14-7.08 (m, 2H), 3.08 (s, 1H), 2.84-2.74 (m, 4H), 1.49 (s, 3H). LCMS (ESI): m/z 477.0 (M + H)⁺ |

### Example 5

### Step 1: (E)-2-Chloro-6-fluoro-4-methoxybenzaldehyde oxime (5a)

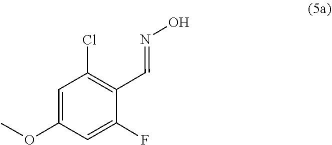

To a solution of 2-chloro-6-fluoro-4-methoxybenzaldehyde (300 mg, 1.59 mmol) in EtOH (5 mL), was added hydroxylamine (0.3 mL). The mixture was stirred at rt for 2 h, concentrated and purified by FCC (PE:EA=10:1) to give compound 5a as a yellow solid. LCMS (ESI): m/z 204.2 (M+H)⁺.

### Step 2: (Z)-2-Chloro-6-fluoro-N-hydroxy-4-methoxybenzimidoyl chloride (5b)

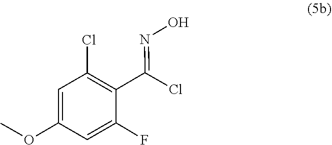

A solution of compound 5a (100 mg, 0.49 mmol) in DMF (3 mL) was slowly added N-chloro-succinimide (79 mg, 0.58 mmol) at 0° C. Then the mixture was stirred at 0° C. for 1 h, diluted with water and extracted with EA (3×30 mL). The combined organic layer was dried over Na₂SO₄ and concentrated to afford compound 5b as a yellow solid, which was used in the next step without further purification.

### Step 3: 4-(4-(3-(2-Chloro-6-fluoro-4-methoxyphenyl)-4-(pyrimidin-2-yl)isoxazol-5-yl)-5-(trifluoro-methyl)-1H-pyrazol-1-yl)-2-methylbutan-4,4-d₂₋₂-ol (5)

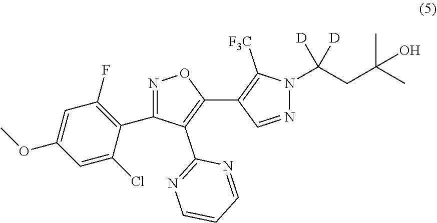 [SMILES: [2H]C([2H])(CC(C)(C)O)n1ncc(-c2onc(-c3c(F)cc(OC)cc3Cl)c2-c2ncccn2)c1C(F)(F)F]

Compound 5b was treated as described in Example 3, step 7 to step 8 to give compound 5 as a white solid. ¹H-NMR (500 MHz, DMSO-d₆) δ 8.64 (d, J=4.5 Hz, 2H), 8.18 (s, 1H), 7.34 (dd, J=4.5, 5.5 Hz, 1H), 7.08-7.03 (m, 2H), 4.57 (br s, 1H), 3.87 (s, 3H), 1.96 (s, 2H), 1.17 (s, 6H). LCMS (ESI): m/z 528.3 (M+H)⁺.

### Example 5/1 to 5/2

The following Examples were prepared as described in Example 5 by using the appropriate building block:

| # | building block | structure | analytical data |
|---|---|---|---|
| 5/1 | 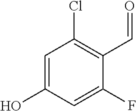 [SMILES: O=Cc1c(F)cc(O)cc1Cl] | 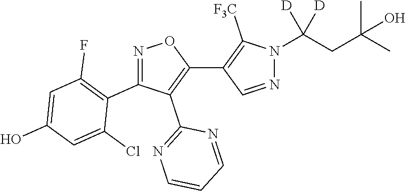 [SMILES: [2H]C([2H])(CC(C)(C)O)n1ncc(-c2onc(-c3c(F)cc(O)cc3Cl)c2-c2ncccn2)c1C(F)(F)F] | LCMS (ESI): m/z 514.1 (M + H)⁺ |
| 5/2 | 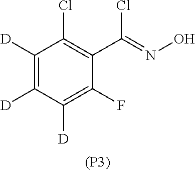 | 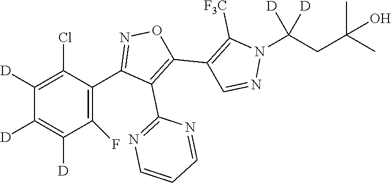 [SMILES: [2H]c1c([2H])c(F)c(-c2noc(-c3cnn(C([2H])([2H])CC(C)(C)O)c3C(F)(F)F)c2-c2ncccn2)c(Cl)c1[2H]] | ¹H-NMR (400 MHz, DMSO-d₆) δ 8.61 (d, J = 4.8 Hz, 2H), 8.21 (s, 1H), 7.34 (t, J = 5.0 Hz, 1H), 4.58 (s, 1H), 1.97 (s, 2H), 1.18 (s, 6H). LCMS (ESI): m/z 501.0 (M + H)⁺ |

### Example 6: 4-(4-(3-(2-Chloro-6-fluoro-4-(2-morpholinoethoxy)phenyl)-4-(pyrimidin-2-yl)isoxazol-5-yl)-5-(trifluoromethyl)-1H-pyrazol-1-yl)-2-methylbutan-4,4-d₂₋₂-ol_(6)

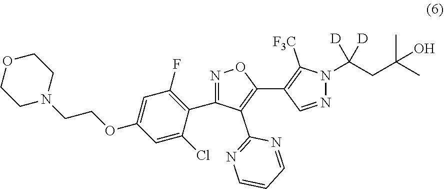 [SMILES: [2H]C([2H])(CC(C)(C)O)n1ncc(-c2onc(-c3c(F)cc(OCCN4CCOCC4)cc3Cl)c2-c2ncccn2)c1C(F)(F)F]

To a solution of compound 5/1 (61 mg, 0.12 mmol) in MeCN (2 mL) was added 4-(2-bromo-ethyl)morpholine (35 mg, 0.18 mmol) and K₂CO₃ (41 mg, 0.30 mmol). The mixture was stirred at rt under N₂ for 2 h, diluted with water (20 mL) and extracted with EA (3×20 mL). The combined organic layer was dried over Na₂SO₄, concentrated and purified by prep-HPLC to give compound 6 as a white solid. ¹H-NMR (400 MHz, DMSO-d₆) δ 8.51 (d, J=5.2 Hz, 2H), 8.19 (s, 1H), 7.35 (t, J=5.0 Hz, 1H), 7.10-7.05 (m, 2H), 4.59 (s, 1H), 4.21 (t, J=5.4 Hz, 2H), 3.59 (t, J=4.0 Hz, 4H), 2.71 (br s, 2H), 2.50 (t, J=1.6 Hz, 4H), 1.96 (s, 2H), 1.17 (s, 6H). LCMS (ESI): m/z 627.1 (M+H)⁺.

### Example 7: Methyl 3-(2-chloro-6-fluorophenyl)-5-(1-(3-hydroxy-3-methylbutyl-1,1-d₂)-5-(tri-fluoromethyl)-1H-pyrazol-4-yl)isoxazole-4-carboxylate (7)

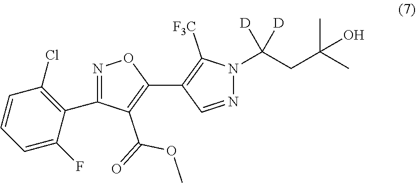 [SMILES: [2H]C([2H])(CC(C)(C)O)n1ncc(-c2onc(-c3c(F)cccc3Cl)c2C(=O)OC)c1C(F)(F)F]

To a solution of Intermediate 3c (0.72 g, 6.0 mmol) and methyl (Z)-3-(2-chloro-6-fluorophenyl)-5-(1-(dimethylamino)-4,4,4-trifluoro-3-oxobut-1-en-2-yl)isoxazole-4-carboxylate (2.5 g, 5.9 mmol) in EtOH (20 mL) was added DIPEA (2.1 mL). The solution was stirred at rt for 6 h, diluted with water and extracted with EA (3×100 mL). The combined organic layer was washed with brine (80 mL), dried over Na₂SO₄, concentrated and purified by FCC (PE:EA=1:1) to give compound 7 as a white solid. ¹H-NMR (500 MHz, DMSO-d₆) δ 8.27 (s, 1H), 7.71-7.65 (m, 1H), 7.56 (d, J=8.5 Hz, 1H), 7.47 (t, J=8.8 Hz, 1H), 4.59 (br s, 1H), 3.58 (s, 3H), 1.97 (s, 2H), 1.18 (s, 6H). LCMS (ESI): m/z 478.2 (M+H)⁺.

### Example 7/1: 3-(2-Chloro-6-fluorophenyl)-5-(1-(3-hydroxy-3-methylbutyl-1,1-d₂)-5-(trifluoro-methyl)-1H-pyrazol-4-yl)isoxazole-4-carboxylic acid (7/1)

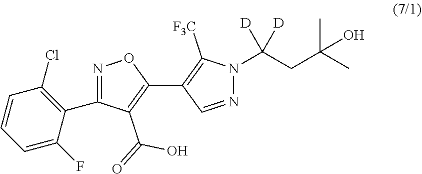 [SMILES: [2H]C([2H])(CC(C)(C)O)n1ncc(-c2onc(-c3c(F)cccc3Cl)c2C(=O)O)c1C(F)(F)F]

To a solution of compound 7 (3.2 g, 6.7 mmol) in MeOH (10 mL) and THF (5 mL) was added 2N NaOH (5 mL) at rt. The mixture was stirred for 8 h at rt, concentrated, adjust to pH=3 with 2N HCl and purified by reversed-phase flash chromatography (C18) (0.1% TFA in water, 10% to 100% MeCN) to afford compound 7/1 as a white solid. LCMS (ESI): m/z 464.0 (M+H)⁺.

### Example 7/2: Methyl-d₃ 3-(2-chloro-6-fluorophenyl)-5-(1-(3-hydroxy-3-methylbutyl-1,1-d₂)-5-(tri-fluoromethyl)-1H-pyrazol-4-yl)isoxazole-4-carboxylate (7/2)

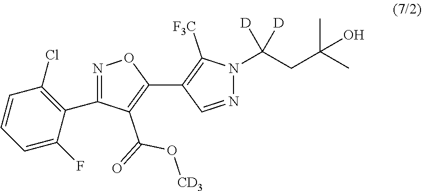 [SMILES: [2H]C([2H])([2H])OC(=O)c1c(-c2c(F)cccc2Cl)noc1-c1cnn(C([2H])([2H])CC(C)(C)O)c1C(F)(F)F]

To a solution of compound 7/1 (2.8 g, 6.0 mmol) in THF (15 mL) was added K₂CO₃ (2.5 g) and CD₃I (1.04 g) at rt. The mixture was stirred at 50° C. overnight, cooled to rt, diluted with water (30 mL) and extracted with EA (3×50 mL). The combined organic layer was washed with brine (30 mL), dried over Na₂SO₄, filtered, concentrated and purified by reversed-phase flash chromatography (C18) (0.1% NH₄HCO₃ in water, 10% to 100% MeCN) to afford compound 7/1 as a colorless oil. ¹H-NMR (400 MHz, DMSO-d₆) δ 8.27 (s, 1H), 7.71-7.65 (m, 1H), 7.56 (d, J=8.4 Hz, 1H), 7.47 (td, J=0.8, 8.6 Hz, 1H), 4.60 (s, 1H), 1.96 (s, 2H), 1.18 (s, 6H). LCMS (ESI): m/z 481.0 (M+H)⁺.

### Example 8: 4-(4-(3-(2-Chloro-6-fluorophenyl)-4-(hydroxymethyl-d₂)isoxazol-5-yl)-5-(trifluoro-methyl)-1H-pyrazol-1-yl)-2-methylbutan-4,4-d₂₋₂-ol (8)

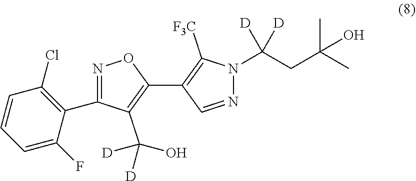 [SMILES: [2H]C([2H])(O)c1c(-c2c(F)cccc2Cl)noc1-c1cnn(C([2H])([2H])CC(C)(C)O)c1C(F)(F)F]

To a solution of compound 7 (0.30 g, 0.63 mmol) in MeOD (5 mL) was added NaBD₄ (53 mg) at 0° C. and the mixture was stirred for 2 h at 0° C. Additional NaBD₄ (53 m) was added and stirring was continued at rt overnight. The mixture was quenched with aq. NH₄Cl (20 mL) and extracted with EA (3×30 mL). The combined organic layer was washed with brine (30 mL), dried over Na₂SO₄, filtered, concentrated and purified by FCC (PE:EA=2:1) to afford compound 8 as a yellow oil. LCMS (ESI): m/z 452.0 (M+H)⁺.

### Example 9

### Step 1: (3-(2-Chloro-6-fluorophenyl)-5-(1-(3-hydroxy-3-methylbutyl-1,1-d₂)-5-(trifluoromethyl)-1H-pyrazol-4-yl)isoxazol-4-yl)methyl-d₂ methanesulfonate (9a)

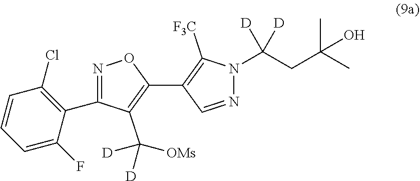 [SMILES: [2H]C([2H])(OS(C)(=O)=O)c1c(-c2c(F)cccc2Cl)noc1-c1cnn(C([2H])([2H])CC(C)(C)O)c1C(F)(F)F]

To a solution of compound 8 (140 mg, 0.31 mmol) in DCM (5 mL) was added triethylamine (88 mg, 0.63 mmol) and MsCl (53 mg, 0.46 mmol) at 0° C. The mixture was stirred at rt for 1h, concentrated and purified by FCC (PE:EA=5:1) to afford compound 9a as a yellow oil. LCMS (ESI): m/z 530.0 (M+H)⁺.

### Step 2: 4-(4-(3-(2-Chloro-6-fluorophenyl)-4-(cyclopropoxymethyl-d₂)isoxazol-5-yl)-5-(trifluoro-methyl)-1H-pyrazol-1-yl)-2-methylbutan-4,4-d₂₋₂-ol (9)

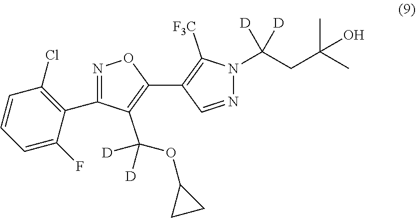 [SMILES: [2H]C([2H])(OC1CC1)c1c(-c2c(F)cccc2Cl)noc1-c1cnn(C([2H])([2H])CC(C)(C)O)c1C(F)(F)F]

To a solution of compound 9a (100 mg, 0.19 mmol) in THF (5 mL) was added cyclopropanol (13 mg, 0.23 mmol) and tert-BuOK (42 mg, 0.37 mmol) at rt. The mixture was stirred at rt overnight, quenched with aq. NH₄Cl (20 mL) and extracted with EA (3×30 mL). The combined organic layer was washed with brine (30 mL), dried over Na₂SO₄, filtered, concentrated and purified by prep-HPLC to afford compound 9 as a yellow solid. ¹H-NMR (400 MHz, DMSO-d₆) δ 8.13 (s, 1H), 7.73-7.63 (m, 1H), 7.58 (d, J=8.0 Hz, 1H), 7.47 (t, J=8.6 Hz, 1H), 4.58 (br s, 1H), 3.15-3.06 (m, 1H), 1.95 (s, 2H), 1.17 (s, 6H), 0.26-0.20 (m, 2H), 0.12-0.05 (m, 2H). LCMS (ESI): m/z 492.0 (M+H)⁺.

### Example 10/1 to 10/3

The following Examples were prepared similar as described above by using the appropriate building blocks.

| # | building block | structure | analytical data |
|---|---|---|---|
| 10/1 | 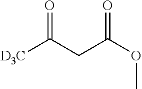 | 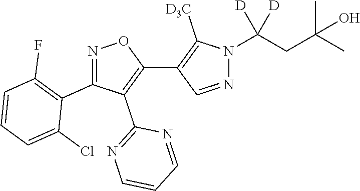 [SMILES: [2H]C([2H])([2H])c1c(-c2onc(-c3c(F)cccc3Cl)c2-c2ncccn2)cnn1C([2H])([2H])CC(C)(C)O] | ¹H-NMR (400 MHz, CD₃OD) δ 8.61 (d, J = 5.2 Hz, 2H), 8.13 (s, 1H), 7.52-7.45 (m, 1H), 7.32 (d, J = 7.6 Hz, 1H), 7.25 (t, J = 5.0 Hz, 1H), 7.19 (t, J = 8.6 Hz, 1H), 1.98 (s, 2H), 1.27 (s, 6H). LCMS (ESI): m/z 447.1 (M + H)⁺ |
| 10/2 | 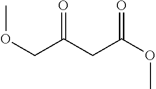 | 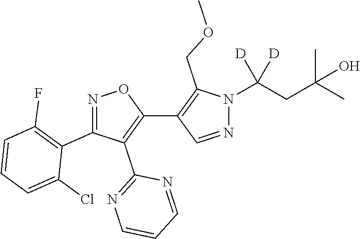 [SMILES: [2H]C([2H])(CC(C)(C)O)n1ncc(-c2onc(-c3c(F)cccc3Cl)c2-c2ncccn2)c1COC] | ¹H-NMR (400 MHz, CD₃OD) δ 8.60 (d, J = 4.4 Hz, 2H), 8.14 (s, 1H), 7.53-7.46 (m, 1H), 7.33 (d, J = 8.4 Hz, 1H), 7.27-7.16 (m, 2H), 4.71 (s, 2H), 3.35 (s, 3H), 2.05 (s, 2H), 1.28 (s, 6H). LCMS (ESI): m/z 474.2 (M + H)⁺ |
| 10/3 | 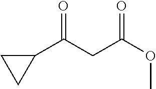 | 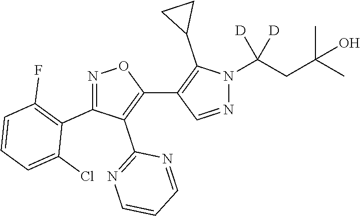 [SMILES: [2H]C([2H])(CC(C)(C)O)n1ncc(-c2onc(-c3c(F)cccc3Cl)c2-c2ncccn2)c1C1CC1] | ¹H-NMR (400 MHz, CD₃OD) δ 8.56 (d, J = 4.8 Hz, 2H), 7.91 (s, 1H), 7.53-7.46 (m, 1H), 7.33 (d, J = 8.4 Hz, 1H), 7.23-7.17 (m, 2H), 2.05 (s, 2H), 1.72-1.65 (m, 1H), 1.29 (s, 6H), 0.85-0.80 (m, 2H), 0.52-0.48 (m, 2H). LCMS (ESI): m/z 470.2 (M + H)⁺ |

### Example 100

### Step 1: 3-(2-Chloro-6-fluorophenyl)-5-(1-(3-chlorophenyl)-5-(trifluoromethyl)-1H-pyrazol-4-yl)isoxazole-4-carboxylic acid (100a)

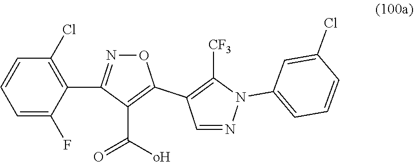

To a solution of methyl 3-(2-chloro-6-fluorophenyl)-5-(1-(3-chlorophenyl)-5-(trifluoromethyl)-1H-pyrazol-4-yl)isoxazole-4-carboxylate (500 mg, 1.00 mmol) in THF (5 mL) was added NaOH (4N, 1 mL) at rt and the mixture was stirred for 4 h, concentrated, adjusted to pH<3 by addition of 1N HCl and extracted with EA (3×30 mL). The combined organic layer was washed with brine (30 mL), dried over Na₂SO₄, filtered, concentrated and purified by FCC (PE:EA=1:1) to afford compound 100a as a white solid. LCMS (ESI): m/z 485.9 (M+H)⁺.

### Step 2: Methyl-d₃ 3-(2-chloro-6-fluorophenyl)-5-(1-(3-chlorophenyl)-5-(trifluoromethyl)-1H-pyrazol-4-yl)isoxazole-4-carboxylate (100)

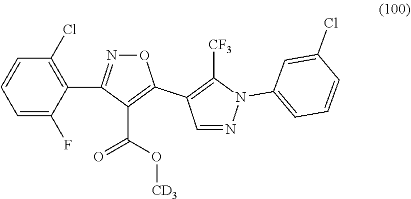 [SMILES: [2H]C([2H])([2H])OC(=O)c1c(-c2c(F)cccc2Cl)noc1-c1cnn(-c2cccc(Cl)c2)c1C(F)(F)F]

To a solution of compound 100a (0.15 g, 0.31 mmol) in THF/MeOD (1:1, 3 mL) was added K₂CO₃ (85 mg) and CD₃I (68 mg) at rt and the mixture was stirred at rt overnight, diluted with water (20 mL) and extracted with EA (3×30 mL). The combined organic layer was washed with brine (30 mL), dried over Na₂SO₄, filtered, concentrated and purified by prep-HPLC to afford compound 100 as a white solid. ¹H-NMR (400 MHz, DMSO-d₆) δ 8.58 (s, 1H), 7.87 (s, 1H), 7.78-7.66 (m, 4H), 7.58 (d, J=8.4 Hz, 1H), 7.49 (t, J=8.6 Hz, 1H). LCMS (ESI): m/z 503.2 (M+H)⁺.

### Example 100/1

The following Example was prepared similar as described for Example 100 by using the appropriate building block.

| # | building block | structure | analytical data |
|---|---|---|---|
| 100/1 | ICD₂CD₃ | 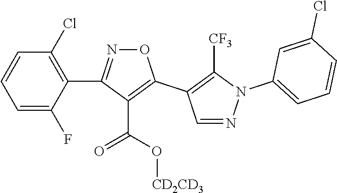 | ¹H-NMR (400 MHz, DMSO-d₆) δ 8.57 (s, 1H), 7.84 (s, 1H), 7.78-7.63 (m, 4H), 7.58 (d, J = 8.4 Hz, 1H), 7.49 (t, J = 8.8 Hz, 1H). LCMS (ESI): m/z 519.0 (M + H)⁺ |

### Example 101: (3-(2-Chloro-6-fluorophenyl)-5-(1-(3-chlorophenyl)-5-(trifluoromethyl)-1H-pyrazol-4-yl)isoxazol-4-yl)methan-d₂-ol (101)

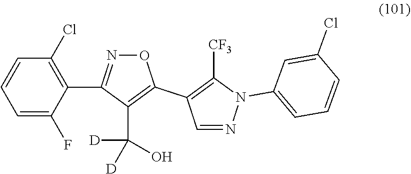 [SMILES: [2H]C([2H])(O)c1c(-c2c(F)cccc2Cl)noc1-c1cnn(-c2cccc(Cl)c2)c1C(F)(F)F]

Starting material methyl 3-(2-chloro-6-fluorophenyl)-5-(1-(3-chlorophenyl)-5-(trifluoromethyl)-1H-pyrazol-4-yl)isoxazole-4-carboxylate was treated similar as described in Example 8 and purified by FCC (PE:EA=2.5:1) to afford compound 101 as a yellow oil. LCMS (ESI): m/z 473.9 (M+H)⁺.

### Example 102: 3-(2-Chloro-6-fluorophenyl)-5-(1-(3-chlorophenyl)-5-(trifluoromethyl)-1H-pyrazol-4-yl)-4-(cyclopropoxymethyl-d₂)isoxazole (102)

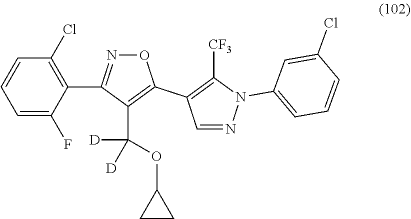 [SMILES: [2H]C([2H])(OC1CC1)c1c(-c2c(F)cccc2Cl)noc1-c1cnn(-c2cccc(Cl)c2)c1C(F)(F)F]

Compound 101 was treated similar as described in Example 9 to afford compound 102 as a yellow oil. ¹H-NMR (400 MHz, DMSO-d₆) δ 8.43 (s, 1H), 7.85 (s, 1H), 7.76-7.66 (m, 4H), 7.59 (d, J=8.0 Hz, 1H), 7.49 (t, J=8.6 Hz, 1H), 3.19-3.14 (m, 1H), 0.30-0.23 (m, 2H), 0.15-0.10 (m, 2H). LCMS (ESI): m/z 514.1 (M+H)⁺.

### Example 102/1 to 102/2

The following Examples were prepared similar as described for Example 102 by using the appropriate building block.

| # | building block | structure | analytical data |
|---|---|---|---|
| 102/1 | 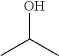 | 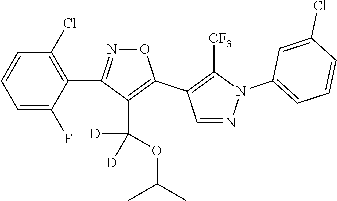 [SMILES: [2H]C([2H])(OC(C)C)c1c(-c2c(F)cccc2Cl)noc1-c1cnn(-c2cccc(Cl)c2)c1C(F)(F)F] | ¹H-NMR (400 MHz, CD₃OD) δ 8.20 (s, 1H), 7.67-7.53 (m, 5H), 7.46 (d, J = 8.4 Hz, 1H), 7.32-7.27 (m, 1H), 3.45 (q, J = 6.0 Hz, 1H), 0.93 (d, J = 6.4 Hz, 6H). LCMS (ESI): m/z 516.1 (M + H)⁺ |
| 102/2 | 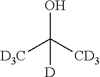 [SMILES: [2H]C([2H])([2H])C([2H])(O)C([2H])([2H])[2H]] | 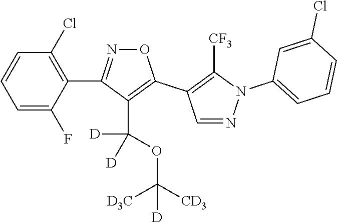 [SMILES: [2H]C([2H])(OC([2H])(C([2H])([2H])[2H])C([2H])([2H])[2H])c1c(-c2c(F)cccc2Cl)noc1-c1cnn(-c2cccc(Cl)c2)c1C(F)(F)F] | ¹H-NMR (400 MHz, CD₃OD) δ 8.21 (s, 1H), 7.68-7.54 (m, 5H), 7.47 (d, J = 8.8 Hz, 1H), 7.32-7.28 (m, 1H). LCMS (ESI): m/z 523.1 (M + H)⁺ |

### Example 103

### 3-(2-Chloro-6-fluorophenyl)-5-(1-(3-chlorophenyl)-5-(trifluoromethyl)-1H-pyrazol-4-yl)-4-(pyrimidin-2-yl-d₃)isoxazole (103)

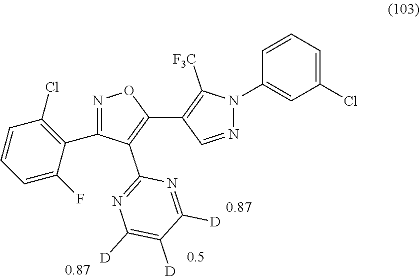 [SMILES: [2H]c1nc(-c2c(-c3c(F)cccc3Cl)noc2-c2cnn(-c3cccc(Cl)c3)c2C(F)(F)F)nc([2H])c1[2H]]

By using intermediate P2 similar as outlined in WO2012/101263, the target compound 103 was prepared as a white solid. According to NMR, the deuteration ratio is as depicted in the chemical formula. ¹H-NMR (400 MHz, CD₃OD) δ 8.60-8.58 (m, 0.27H), 8.29 (s, 1H), 7.68-7.50 (m, 5H), 7.37 (dd, J=0.8, 8.0 Hz, 1H), 7.26-2.21 (m, 1.5H). LCMS (ESI): m/z 523.8 (M+H)⁺.

### Example 104: Methyl 3-(2-chloro-6-fluorophenyl-3,4,5-d₃)-5-(1-(3-chlorophenyl)-5-(trifluoro-methyl)-1H-pyrazol-4-yl)isoxazole-4-carboxylate (104)

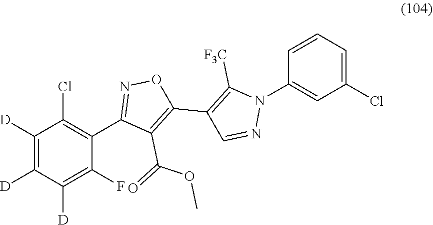 [SMILES: [2H]c1c([2H])c(F)c(-c2noc(-c3cnn(-c4cccc(Cl)c4)c3C(F)(F)F)c2C(=O)OC)c(Cl)c1[2H]]

By using intermediate P3 similar as described above or in WO2012/101261 the target compound 104 was prepared. ¹H-NMR (400 MHz, DMSO-d₆) δ 8.58 (s, 1H), 7.87 (s, 1H), 7.77-7.74 (m, 1H), 7.69-7.67 (m, 2H), 3.66 (s, 3H). LCMS (ESI): m/z 503.0 (M+H)⁺.

### Example 105: Methyl 3-(2-chloro-6-fluorophenyl)-5-(1-(3-chlorophenyl-2,4,6-d₃)-5-(trifluoro-methyl)-1H-pyrazol-4-yl)isoxazole-4-carboxylate (105)

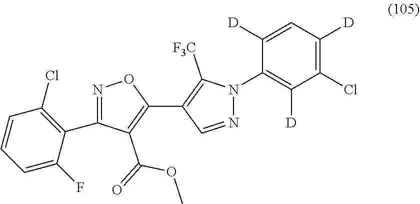 [SMILES: [2H]c1cc([2H])c(-n2ncc(-c3onc(-c4c(F)cccc4Cl)c3C(=O)OC)c2C(F)(F)F)c([2H])c1Cl]

Using intermediate P4 the target compound 105 was prepared similar as outlined in WO2012/101261. ¹H-NMR (400 MHz, DMSO-d₆) δ 8.58 (s, 1H), 7.72-7.66 (m, 2H), 7.58 (d, J=8.0 Hz, 1H), 7.51 (t, J=8.8 Hz, 1H), 3.66 (s, 3H). LCMS (ESI): 502.6 m/z (M+H)⁺.

### Example 106: 3-(2-Chloro-6-fluorophenyl)-5-(1-(3-chlorophenyl-2,4,6-d₃)-5-(trifluoromethyl)-1H-pyrazol-4-yl)-4-(thiazol-2-yl)isoxazole (106)

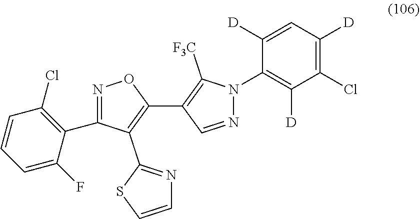 [SMILES: [2H]c1cc([2H])c(-n2ncc(-c3onc(-c4c(F)cccc4Cl)c3-c3nccs3)c2C(F)(F)F)c([2H])c1Cl]

Using intermediate P4 the target compound 106 was prepared similar as outlined in WO2012/101263. ¹H-NMR (400 MHz, DMSO-d₆) δ 8.62 (s, 1H), 7.87 (d, J=3.2 Hz, 1H), 7.77-7.72 (m, 2H), 7.68 (s, 1H), 7.61 (d, J=8.4 Hz, 1H), 7.53 (t, J=8.6 Hz, 1H). LCMS (ESI): 508.0 m/z (M+H)⁺.

### Example 107: 3-(2-Chloro-6-fluorophenyl)-5-(1-(3-chlorophenyl)-5-(trifluoromethyl)-1H-pyrazol-4-yl)-4-(thiazol-2-yl-d₂)isoxazole (107)

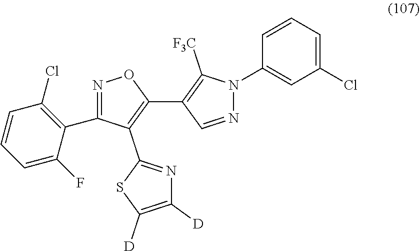 [SMILES: [2H]c1nc(-c2c(-c3c(F)cccc3Cl)noc2-c2cnn(-c3cccc(Cl)c3)c2C(F)(F)F)sc1[2H]]

Using intermediate P1 the target compound 107 was be prepared similar as outlined in WO2012/101263. ¹H-NMR (400 MHz, DMSO-d₆) δ 8.62 (s, 1H), 7.86 (s, 1H), 7.78-7.67 (m, 4H), 7.61 (d, J=8.4 Hz, 1H), 7.53 (t, J=8.6 Hz, 1H). LCMS (ESI): m/z 527.0 (M+H)⁺.

### Example 108

### Step 1: (3-(2-Chloro-6-fluorophenyl)-5-(1-(3-chlorophenyl)-5-(trifluoromethyl)-1H-pyrazol-4-yl)isoxazol-4-yl)methyl-d₂ methanesulfonate (108a)

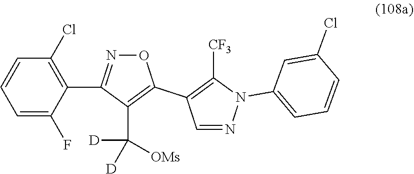 [SMILES: [2H]C([2H])(OS(C)(=O)=O)c1c(-c2c(F)cccc2Cl)noc1-c1cnn(-c2cccc(Cl)c2)c1C(F)(F)F]

To a solution of compound 101 (10 g, 21 mmol) in CH₂Cl₂ (50 mL) was added NEt₃ (6.4 g) and MsCl (2.9 g) at 0° C. The mixture was stirred for 1 h at rt, concentrated and purified by FCC (PE:EA=5:1) to afford compound 108a as a yellow oil. LCMS (ESI): 551.6 m/z (M+H)⁺.

### Step 2: 3-(2-Chloro-6-fluorophenyl)-5-(1-(3-chlorophenyl)-5-(trifluoromethyl)-1H-pyrazol-4-yl)-4-(iodomethyl-d₂)isoxazole (108)

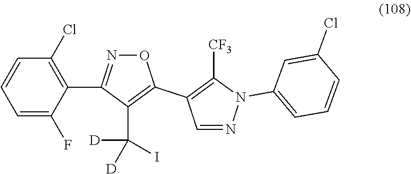 [SMILES: [2H]C([2H])(I)c1c(-c2c(F)cccc2Cl)noc1-c1cnn(-c2cccc(Cl)c2)c1C(F)(F)F]

To a solution of compound 108a (9.0 g, 16 mmol) in dimethoxyethane (50 mL) was added KI (5.42 g). The mixture was stirred at 80° C. for 5 h, cooled to rt, diluted with water and extracted with EA (3×50 mL). The combined organic layer was washed with brine (50 mL), dried over Na₂SO₄, concentrated and purified by FCC (PE:EA=5:1) to give compound 108 as a white solid. LCMS (ESI): 585.0 m/z (M+H)⁺.

### Example 109: 3-(2-Chloro-6-fluorophenyl)-5-(1-(3-chlorophenyl)-5-(trifluoromethyl)-1H-pyrazol-4-yl)-4-((trifluoromethoxy)methyl-d₂)isoxazole (109)

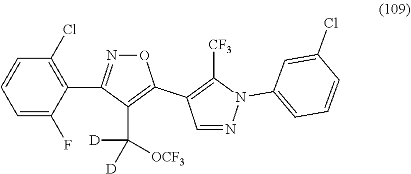 [SMILES: [2H]C([2H])(OC(F)(F)F)c1c(-c2c(F)cccc2Cl)noc1-c1cnn(-c2cccc(Cl)c2)c1C(F)(F)F]

Inside the glovebox, compound 108 (150 mg, 0.26 mmol) was weighed into a 4 mL amber vial and dissolved in anhydrous acetonitrile (0.5 mL). This solution was stirred at −30° C. for 5 min, then AgOCF₃ (1.0M in CH₃CN, 1.2 mL; prepared as described in Chem. Eur. J. 2020; 26:2183) was added at once into the vial. After stirring the mixture at rt for 12 h, the mixture was purified by reversed-phase flash chromatography (C18) (0.1% NH₄HCO₃ in water, 10% to 100% MeCN) to afford compound 109 as a white solid. ¹H-NMR (400 MHz, CD₃OD) δ 8.17 (s, 1H), 7.69-7.55 (m, 5H), 7.50 (d, J=8.4 Hz, 1H), 7.35-7.31 (m, 1H). LCMS (ESI): 542.0 m/z (M+H)⁺.

### Example 110: 3-(2-Chloro-6-fluorophenyl)-5-(1-(3-chlorophenyl)-5-(trifluoromethyl)-1H-pyrazol-4-yl)-4-((methoxy-d₃)methyl-d₂)isoxazole (110)

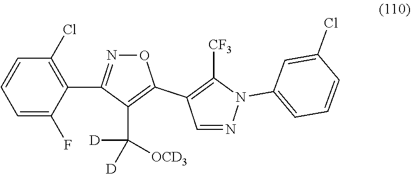

To a solution of compound 101 (200 mg, 0.42 mmol) in THF (5 mL) was added CD₃I (123 mg) and tert-BuOK (71 mg) at rt. The mixture was stirred at rt overnight, diluted with aq. NH₄Cl (20 mL) and extracted with EA (3×30 mL). The combined organic layer was washed with brine (30 mL), dried over Na₂SO₄, filtered, concentrated and purified by prep-HPLC to afford compound 110 as yellow oil. ¹H-NMR (400 MHz, CD₃OD) δ 8.20 (s, 1H), 7.68-7.54 (m, 5H), 7.47 (d, J=8.0 Hz, 1H), 7.33-7.28 (m, 1H). LCMS (ESI): 491.0 m/z (M+H)⁺.

### Example 110/1 to 110/2

The following Examples were prepared similar as described for Example 110 by using the appropriate building block.

| # | building block | structure | analytical data |
|---|---|---|---|
| 110/1 | ICD₂CD₃ | 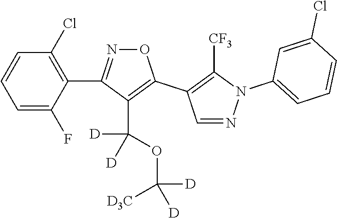 [SMILES: [2H]C([2H])(OC([2H])([2H])C([2H])([2H])[2H])c1c(-c2c(F)cccc2Cl)noc1-c1cnn(-c2cccc(Cl)c2)c1C(F)(F)F] | ¹H-NMR (400 MHz, CD₃OD) δ 8.20 (s, 1H), 7.68-7.54 (m, 5H), 7.47 (d, J = 8.4 Hz, 1H), 7.32-7.28 (m, 1H). LCMS (ESI): m/z 507.1 (M + H)⁺ |
| 110/2 | 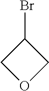 | 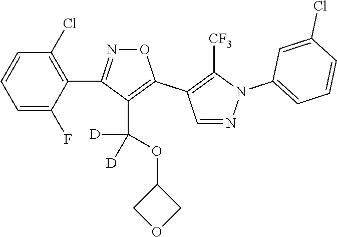 [SMILES: [2H]C([2H])(OC1COC1)c1c(-c2c(F)cccc2Cl)noc1-c1cnn(-c2cccc(Cl)c2)c1C(F)(F)F] | ¹H-NMR (400 MHz, CD₃OD) δ 8.23 (s, 1H), 7.70-7.56 (m, 5H), 7.51 (d, J = 8.0 Hz, 1H), 7.37-7.32 (m, 1H), 4.62 (t, J = 6.6 Hz, 2H), 4.50-4.44 (m, 1H), 4.30-4.26 (m, 2H). LCMS (ESI): m/z 530.0 (M + H)⁺ |
| 110/3 | ICH₂CH₃ | 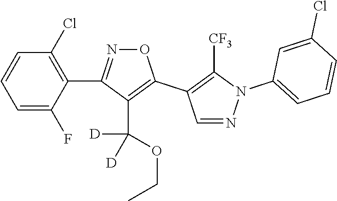 [SMILES: [2H]C([2H])(OCC)c1c(-c2c(F)cccc2Cl)noc1-c1cnn(-c2cccc(Cl)c2)c1C(F)(F)F] | ¹H-NMR (400 MHz, DMSO-d₆) δ 8.41 (s, 1H), 7.84 (d, J = 2.0 Hz, 1H), 7.76-7.64 (m, 4H), 7.58 (d, J = 8.0 Hz, 1H), 7.50-7.45 (m, 1H), 3.24 (q, J = 6.8 Hz, 2H), 0.98 (t, J = 6.8 Hz, 3H). LCMS (ESI): m/z 502.2 (M + H)⁺ |

### Example 111/1 to 111/4

The following Examples was prepared similar as described above in Example 5, 101 and 108 and by using the appropriate building block.

| # | building block | structure | analytical data |
|---|---|---|---|
| 111/1 | 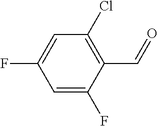 [SMILES: O=Cc1c(F)cc(F)cc1Cl] | 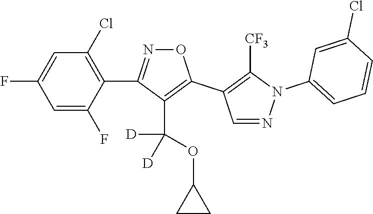 [SMILES: [2H]C([2H])(OC1CC1)c1c(-c2c(F)cc(F)cc2Cl)noc1-c1cnn(-c2cccc(Cl)c2)c1C(F)(F)F] | ¹H-NMR (500 MHz, DMSO-d₆) δ 8.42 (s, 1H), 7.85 (s, 1H), 7.76-7.62 (m, 5H), 3.19- 3.16 (m, 1H), 0.31-0.27 (m, 2H), 0.18-0.15 (m, 2H). LCMS (ESI): m/z 532.0 (M + H)⁺ |
| 111/2 | 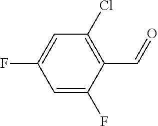 [SMILES: O=Cc1c(F)cc(F)cc1Cl] | 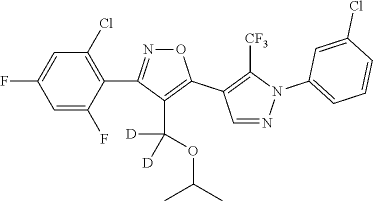 [SMILES: [2H]C([2H])(OC(C)C)c1c(-c2c(F)cc(F)cc2Cl)noc1-c1cnn(-c2cccc(Cl)c2)c1C(F)(F)F] | ¹H-NMR (500 MHz, CD₃OD) δ 8.20 (s, 1H), 7.67-7.54 (m, 4H), 7.39-7.37 (m, 1H), 7.27-7.22 (m, 1H), 3.47 (q, J = 6.0 Hz, 1H), 0.96 (d, J = 6.5 Hz, 6H). LCMS (ESI): m/z 533.9 (M + H)⁺ |
| 111/3 | 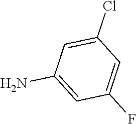 | 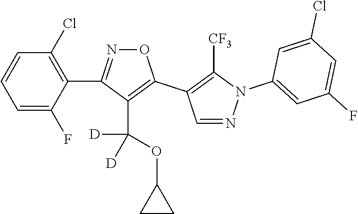 [SMILES: [2H]C([2H])(OC1CC1)c1c(-c2c(F)cccc2Cl)noc1-c1cnn(-c2cc(F)cc(Cl)c2)c1C(F)(F)F] | ¹H-NMR (500 MHz, DMSO-d₆) δ 8.45 (s, 1H), 7.84-7.67 (m, 4H), 7.59 (d, J = 8.0 Hz, 1H), 4.49 (t, J = 8.8 Hz, 1H), 3.17-3.14 (m, 1H), 0.28-0.25 (m, 2H), 0.13-0.10 (m, 2H). LCMS (ESI): m/z 532.2 (M + H)⁺ |
| 111/4 | 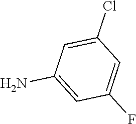 | 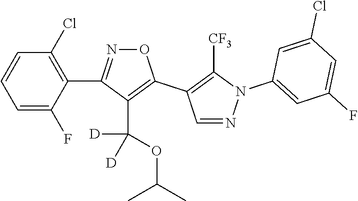 [SMILES: [2H]C([2H])(OC(C)C)c1c(-c2c(F)cccc2Cl)noc1-c1cnn(-c2cc(F)cc(Cl)c2)c1C(F)(F)F] | ¹H-NMR (500 MHz, DMSO-d₆) δ 8.44 (s, 1H), 7.84-7.65 (m, 4H), 7.48 (d, J = 9.0 Hz, 1H), 7.48-7.44 (m, 1H), 3.39-3.34 (m, 1H), 0.93 (d, J = 6.0 Hz, 6H). LCMS (ESI): m/z 534.0 (M + H)⁺ |

### Example 200

The in vitro inhibition of RORγt was measured using Indigo Biosciences Human RAR-related Orphan Receptor, Gamma Reporter Assay System (Product #IB304001-32) as described in the technical manual. In brief, 200 μL of Reporter Cells is dispensed into wells of the assay plate and preincubated for 4-6 h. Following the pre-incubation period, culture media are discarded and 200 L/well of the prepared 1×-concentration treatment media are added. Following 22-24 h incubation, treatment media are discarded and Luciferase Detection Reagent is added. The intensity of light emission (in units of ‘Relative Light Units’; RLU) from each assay well is quantified using a plate-reading luminometer. Average values of RLU were determined for each treatment concentration. Non-linear regression analyses were performed and IC₅₀ values determined using GraphPad Prism software. Provided inverse-agonists ursolic acid served as positive control. The following results were obtained:

| Example # | IC₅₀ range |
|---|---|
| 1 | ++ |
| 1/1 | ++ |
| 2-1 | + |
| 2-2 | ++ |
| 2-1/1 | + |
| 2-2/1 | ++ |
| 3 | ++ |
| 3-1 | +++ |
| 3-2 | +++ |
| 3-3 | ++ |
| 4 | ++ |
| 4-1/1 | + |
| 4-2/1 | ++ |
| 5 | + |
| 5/2 | +++ |
| 6 | + |
| 7 | ++ |
| 7/2 | ++ |
| 9 | ++ |
| 10/1 | + |
| 10/2 | + |
| 10/3 | + |
| 100 | +++ |
| 100/1 | +++ |
| 102 | ++ |
| 102/1 | +++ |
| 102/2 | +++ |
| 103 | ++ |
| 104 | ++ |
| 105 | +++ |
| 106 | +++ |
| 107 | ++ |
| 109 | ++ |
| 110 | +++ |
| 110/1 | ++ |
| 110/2 | ++ |
| 110/3 | ++ |
| 111/1 | + |
| 111/2 | + |
| 111/3 | ++ |
| 111/4 | ++ |

IC₅₀ ranges for the RORγt assay as described herein: +++: <10 nM; ++: 10 nM to <100 nM; +: 100 nM to <1 μM; 0: ≥1 μM.

### Example 201: Microsomal Stability

Example 3 and the non-deuterated matched pair from WO2019/048541 (Comparative Example C3) were incubated using three different batches of pooled rat, mouse and human liver microsomes, respectively, for a period of 60 min. The conversion to the metabolite was monitored by HPLC-MS/MS. Verapamil served as positive control. The intrinsic clearance was calculated from the measured remaining compound values (in duplicate) at 0, 10, 30 and 60 minutes. The data points for 60 minutes are as follows:

| | 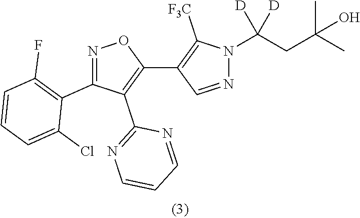 [SMILES: [2H]C([2H])(CC(C)(C)O)n1ncc(-c2onc(-c3c(F)cccc3Cl)c2-c2ncccn2)c1C(F)(F)F] rat liver microsomes batch | | | 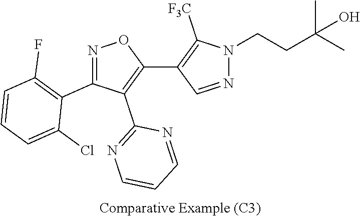 [SMILES: CC(C)(O)CCn1ncc(-c2onc(-c3c(F)cccc3Cl)c2-c2ncccn2)c1C(F)(F)F] mouse liver microsomes batch | | |
|---|---|---|---|---|---|---|
| | | | | MDO | | |
| Clᵢₙₜ | | | | female | 4338001 | 0010003 |
| (μl/min/mg | 4085002 | 9078001 | 1910100 | (Bioreclamation | female | male |
| protein) | (Corning) | (Corning) | (Xenotech) | IVT) | (Corning) | (Corning) |
| Comparative | 140.5 | 131.7 | 133.2 | 100.9 | 90.8 | 94.4 |
| Example C3 | | | | | | |
| Example 3 | 29.7 | 30.2 | 31.4 | 50.3 | 43.9 | 24.0 |
| Verapamil | 258.6 | 217.4 | 168.8 | 350.2 | 176.2 | 304.7 |

| | human liver microsomes batch | | |
|---|---|---|---|
| | 38296 | 1210270 | 1210079 |
| Clᵢₙₜ | mixed gender | male 10- | female 10- |
| (μl/min/mg | 150-donor-pool | donor-pool | donor-pool |
| protein) | (Corning) | (Xenotech) | (Xenotech) |
| Comparative | 12.1 | 6.1 | 6.7 |
| Example C3 | | | |
| Example 3 | 2.2 | 1.1 | 0.0 |
| Verapamil | 190.2 | 131.5 | 115 . . . 8 |
| - | | | |

Conclusion: As exemplified with Example 3, by deuteration of the 1-alkyl-position at the pyrazole moiety the intrinsic clearance in compounds of the present invention can be reduced in rat, mouse and human microsomes compared to the non-deuterated matched pair. A reduced intrinsic clearance is beneficial since it prolongs the residence time of the drug in the body.

Example 100 and the non-deuterated matched pair (Comparative Example C100) were incubated in mouse liver microsomes for a period of 60 min. The metabolism was monitored by HPLC-MS/MS.

Verapamil served as positive control. The intrinsic clearance was calculated from the measured remaining compound values. The data points for 60 minutes are as follows:

| Clᵢₙₜ (μl/min/mg protein) | 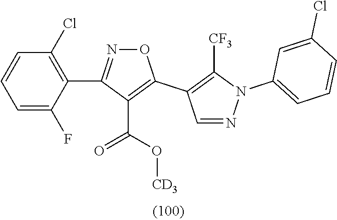 [SMILES: [2H]C([2H])([2H])OC(=O)c1c(-c2c(F)cccc2Cl)noc1-c1cnn(-c2cccc(Cl)c2)c1C(F)(F)F] 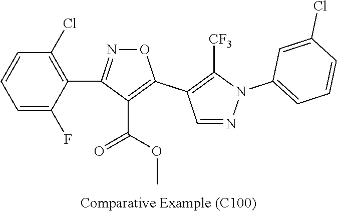 [SMILES: COC(=O)c1c(-c2c(F)cccc2Cl)noc1-c1cnn(-c2cccc(Cl)c2)c1C(F)(F)F] mouse liver microsomes 10003 CD-1, male (Corning) |
|---|---|
| Comparative | 44.6 |
| Example C100 | |
| Example 100 | 24.4 |
| Verapamil | 334.4 |

Conclusion: As exemplified with Example 100, by deuteration of ester moiety the intrinsic clearance in compounds of the present invention can be reduced to a high extent in mouse microsomes compared to the non-deuterated matched pair. A reduced intrinsic clearance is beneficial since it prolongs the residence time of the drug in the body.

Example 102 and the non-deuterated matched pair (Comparative Example C102) were incubated in rat (RLM) and human liver microsomes (HLM), respectively, for a period of 60 min. The metabolism was monitored by HPLC-MS/MS. Verapamil served as positive control. The intrinsic clearance was calculated from the measured remaining compound values. The data points for 60 minutes are as follows:

| Clᵢₙₜ (μl/min/mg protein) | 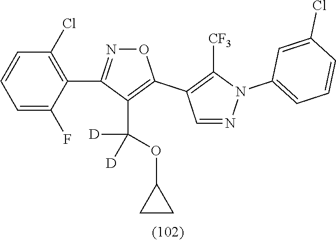 [SMILES: [2H]C([2H])(OC1CC1)c1c(-c2c(F)cccc2Cl)noc1-c1cnn(-c2cccc(Cl)c2)c1C(F)(F)F] rat liver microsomes 9078001 (Corning) | 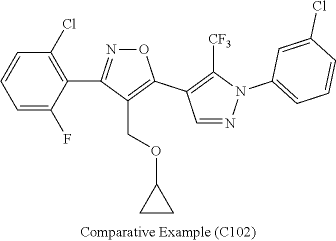 [SMILES: Fc1cccc(Cl)c1-c1noc(-c2cnn(-c3cccc(Cl)c3)c2C(F)(F)F)c1COC1CC1] human liver microsomes 38297 mixed gender 150-donor-pool (Corning) |
|---|---|---|
| Comparative | 32.3 | 11.2 |
| Example C102 | | |
| Example 102 | 29.4 | 6.7 |
| Verapamil | 211.7 | 134.9 |

Conclusion: As exemplified with Example 102, by deuteration of the 4-alkyl-position at the oxazole moiety the intrinsic clearance in compounds of the present invention can be reduced in rat and human microsomes compared to the non-deuterated matched pair. A reduced intrinsic clearance is beneficial since it prolongs the residence time of the drug in the body.

Example 1/1 and the matched pair without alpha-deuteration compared to the pyrazole moiety (Comparative Example C1/1 and C1/2, respectively) were incubated in human liver microsomes for a period of 60 min. The metabolism was monitored by HPLC-MS/MS. Verapamil served as positive control. The intrinsic clearance was calculated from the measured remaining compound values. The data points for 60 minutes are as follows:

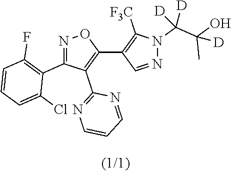 [SMILES: [2H]C(C)(O)C([2H])([2H])n1ncc(-c2onc(-c3c(F)cccc3Cl)c2-c2ncccn2)c1C(F)(F)F]

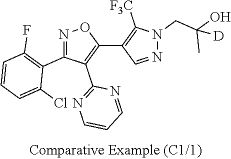 [SMILES: [2H]C(C)(O)Cn1ncc(-c2onc(-c3c(F)cccc3Cl)c2-c2ncccn2)c1C(F)(F)F]

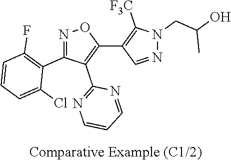 [SMILES: CC(O)Cn1ncc(-c2onc(-c3c(F)cccc3Cl)c2-c2ncccn2)c1C(F)(F)F]

| Clᵢₙₜ | human liver microsomes |
|---|---|
| (μl/min/mg protein) | 38291 mixed gender (Corning) |
| Comparative Example C1/1 | 10.5 |
| Comparative Example C1/2 | 15.7 |
| Example 1/1 | 7.8 |
| Verapamil | 64.2 |

Conclusion: As exemplified with Example 1/1, by deuteration of the alphaalkyl-position at the pyrazole moiety the intrinsic clearance in compounds of the present invention can be reduced in human microsomes compared to the non-deuterated matched pairs. The effect of a deuteration of the betaalkyl-position (mentioned in WO2019/048541) as in Comparative Example C1/1 is less advantageous and may stabilize more the oxidation of the adjacent alcohol.

### Example 202: Rat Pharmacokinetics

The pharmacokinetics of the deuterated compounds of the present invention was evaluated in 3 male and 3 female rats (strain Wistar RjHAN, 7-8 week old) after oral or intravenous cassette dosing to asses the oral bioavailability. Rats are provided with a catheter in the jugular vein (2-3 days prior to blood sampling). At each designated time point (1, 2, 4, 8 and 24 h after dosing), 100 μL blood were collected into Li-heparin tubes, stored on ice until centrifugation (10 minutes at 3000 g, 4° C.) and plasma was prepared within 45 min after collection, frozen at −20° C. and stored at this temperature until processed for LC-MS analysis. The obtained data is as follows:

| Gender | male | | female | |
|---|---|---|---|---|
| Test item | Comparative Example C3 | | | |
| Dose route | po | iv | po | iv |
| Vehicle | po: 5% solutol/95% NaCl 0.9%, iv: 5% solutol/5% EtOH/95% NaCl 0.9% | | | |
| Dosage (mg/kg) | 5 | 1 | 5 | 1 |
| Volume (ml/kg) | 5 | 0.5 | 5 | 0.5 |
| Cₘₐₓ (ng/ml) | 123.4 | — | 473.6 | — |
| C₀ (ng/ml) | — | 2506 | — | 2129 |
| t_{1/2z} (h) | 1.8 | 7.8 | 3.2 | 6.6 |
| AUC_{0-tz} (ng*h/ml) | 287 | 3736 | 1695 | 3607 |
| AUC_{0-∞} (ng*h/ml) | 302 | 3991 | 1705 | 3719 |
| V_{z/f} (ml/kg) | 5 | 6 | 1 | 3 |
| CL_{/f} (ml/(h*kg)) | 44498 | 3537 | 14099 | 3045 |
| Bioavailability (%) | 1.5 | — | 9.4 | — |
| Gender | male | | female | |
| Test item | Example 3 | | | |
| Dose route | po | iv | po | iv |
| Vehicle | po: 5% solutol/95% NaCl 0.9%, iv: 5% solutol/5% EtOH/95% NaCl 0.9% | | | |
| Dosage (mg/kg) | 5 | 1 | 5 | 1 |
| Volume (ml/kg) | 5 | 0.5 | 5 | 0.5 |
| Cₘₐₓ (ng/ml) | 215 | — | 629 | — |
| C₀ (ng/ml) | — | 2465 | — | 2089 |
| t_{1/2z} (h) | 1.9 | 7.2 | 3.2 | 6.6 |
| AUC_{0-tz} (ng*h/ml) | 649 | 3724 | 2931 | 3657 |
| AUC_{0-∞} (ng*h/ml) | 692 | 3973 | 2949 | 3783 |
| V_{z/f} (ml/kg) | 6 | 6 | 1 | 3 |
| CL_{/f} (ml/(h*kg)) | 20566 | 3161 | 7892 | 2887 |
| Bioavailability (%) | 3.5 | — | 16.0 | — |

Abbreviations:
Cₘₐₓ = peak plasma concentration,
t_{1/2} = elimination half-life,
AUC = area under the curve (integral of the concentration-time curve from 0 to 24 h,
CL = clearance,
V_{z/f} = volume of distribution

Conclusion: The non-deuterated Comparative Example C3 itself has a low bioavailability, which can be dramatically improved with statistical significance (p<0.05 in the Tukey's Multiple Comparison Test for AUC_{0-tz} and bioavailability in both genders) by selective deuteration (Example 3).

### Example 203: Mouse Pharmacokinetics

The pharmacokinetics of the compounds of the present invention was evaluated in 3 male and 3 female mice (C57BL/6J, 8 week old) after oral or intravenous cassette dosing to assess the oral bioavailability. Dose was 5 mg/kg (oral) and 1 mg/kg (intravenous), application volume was 5 mL/kg (oral) and 0.5 mL/kg (intravenous), vehicle was 5% solutol, 95% NaCl solution (at 0.9% saline concentration) for oral and 5% solutol, 5% Ethanol, 90% NaCl solution (at 0.9% saline concentration) for intravenous. At each designated time point (0.25, 0.5, 1, 2, 4, 8 and 24 h after dosing), 20 μL whole blood were collected from the tail vein into Li-heparin tubes, frozen on dry ice within 1-2 minutes of sampling and stored at −20° C. until processed for LC-MS analysis. The obtained data is as follows:

| Example # | Cₘₐₓ | | t_{1/2} | | AUC_{0-24 h} | | F |
|---|---|---|---|---|---|---|---|
| Comparative | ♂ 218 | ng/mL | ♂ 2.3 | h | ♂ 431 | ng*h/mL | ♂ 32% |
| Example C3 | ♀ 159 | ng/mL | ♀ 1.4 | h | ♀ 332 | ng*h/mL | ♀ 11% |
| 3 | ♂ 392 | ng/mL | ♂ 2.3 | h | ♂ 1040 | ng*h/mL | ♂ 93% |
| | ♀ 301 | ng/mL | ♀ 1.5 | h | ♀ 771 | ng*h/mL | ♀ 34% |

Abbreviations:
♂ = male,
♀ = female,
Cₘₐₓ = peak plasma concentration,
t_{1/2} = elimination half-life,
AUC_{0-24 h} = area under the curve (integral of the concentration-time curve from 0 to 24 h),
F = bioavailability (systemically available fraction)

Again, the non-deuterated Comparative Example C3 has a low bioavailability, which can be dramatically improved with statistical significance (p<0.01 in the Paired t test for AUC in both genders) by selective deuteration (Example 3).

The pharmacokinetics of additional compounds of the present invention was evaluated in 3 female mice (C57BL/6J, 8 week old) after oral cassette dosing. Dose was 5 mg/kg, application volume was 5 mL/kg, vehicle was 5% solutol, 95% NaCl solution (at 0.9% saline concentration). At each designated time point (0.5, 1, 2, 4, 8 and 24 h after dosing), 20 μL whole blood were collected from the tail vein into Li-heparin tubes, frozen on dry ice within 1-2 minutes of sampling and stored at −20° C. until processed for LC-MS analysis. The obtained data is as follows:

| Example # | Cₘₐₓ | | t_{1/2} | AUC_{0-24 h} |
|---|---|---|---|---|
| Comparative | 26.7 | ng/mL | 5.5 h | 102 ng*h/mL |
| Example C100 | | | | |
| 100 | 50.5 | ng/mL | 6.4 h | 201 ng*h/mL |
| Comparative | 162 | ng/mL | 7.7 h | 571 ng*h/mL |
| Example C102 | | | | |
| 102 | 272 | ng/mL | 7.2 h | 970 ng*h/mL |

Again, the non-deuterated Comparative Example C100 and C102 have lower Cₘₐₓ and AUC values, which can be dramatically improved by selective deuteration (Example 100 and Example 102, respectively).

The pharmacokinetics of additional compounds of the present invention was evaluated in 3 female mice (C57BL/6J, 8 week old) after oral cassette dosing. Dose was 5 mg/kg, application volume was 5 mL/kg, vehicle was 5% solutol, 95% NaCl solution (at 0.9% saline concentration). At each designated time point (0.25, 0.5, 1, 2, 4 and 8 h after dosing), 20 μL whole blood were collected from the tail vein into Li-heparin tubes, frozen on dry ice within 1-2 minutes of sampling and stored at −20° C. until processed for LC-MS analysis. The obtained data is as follows:

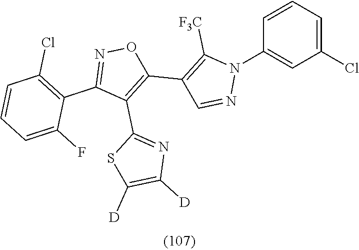 [SMILES: [2H]c1nc(-c2c(-c3c(F)cccc3Cl)noc2-c2cnn(-c3cccc(Cl)c3)c2C(F)(F)F)sc1[2H]]

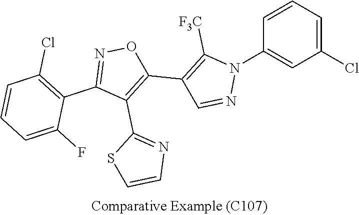 [SMILES: Fc1cccc(Cl)c1-c1noc(-c2cnn(-c3cccc(Cl)c3)c2C(F)(F)F)c1-c1nccs1]

| Example # | Cₘₐₓ | t_{1/2} | AUC_{0-8 h} |
|---|---|---|---|
| Comparative | 357 nM | 1.9 h | 796 ng*h/mL |
| Example C107 | | | |
| 107 | 418 nM | 1.6 h | 871 ng*h/mL |

This experiment shows that deuteration in the thiazole moiety (Example 107) only has a minor effect on the PK properties when compared with the matched pair (Comparative Example C107).

The pharmacokinetics of additional compounds of the present invention was evaluated in 3 female mice (C57BL/6J, 8 week old) after oral cassette dosing. Dose was 5 mg/kg, application volume was 5 mL/kg, vehicle was 5% solutol, 95% NaCl solution (at 0.9% saline concentration). At each designated time point (0.25, 0.5, 1, 2, 4 and 8 h after dosing), 20 μL whole blood were collected from the tail vein into Li-heparin tubes, frozen on dry ice within 1-2 minutes of sampling and stored at −20° C. until processed for LC-MS analysis. The obtained data is as follows:

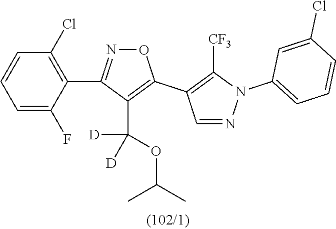 [SMILES: [2H]C([2H])(OC(C)C)c1c(-c2c(F)cccc2Cl)noc1-c1cnn(-c2cccc(Cl)c2)c1C(F)(F)F]

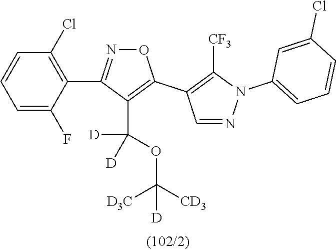 [SMILES: [2H]C([2H])(OC([2H])(C([2H])([2H])[2H])C([2H])([2H])[2H])c1c(-c2c(F)cccc2Cl)noc1-c1cnn(-c2cccc(Cl)c2)c1C(F)(F)F]

| Example # | Cₘₐₓ | t_{1/2} | AUC_{0-8 h} |
|---|---|---|---|
| 102/1 | 922 ng/ml | 2.6 h | 2350 ng*h/mL |
| 102/2 | 112 ng/ml | 2.8 h | 280 ng*h/mL |

This experiment shows that further deuteration at the non-benzyl position of the ether moiety (Example 102/2) surprisingly does not improve PK properties (i.e. dramatically lower AUC value) when compared with the matched pair (Example 102/1).

### Example 204: Antiviral Activity on SARS-CoV-2

The assay for viral replication (YFP) and the cell viability assay has been described in general in Pathogens 2021; 10:1076 and applied to compounds of the present invention furnished the following results:

| Example # | EC₅₀ range | CC₅₀ [μM] |
|---|---|---|
| 1 | +++ | 22 |
| 3 | +++ | 22 |
| 3-1 | +++ | 17 |
| 4 | +++ | >100 |
| 5 | ++ | >100 |
| 6 | ++ | 27 |
| 7/2 | +++ | 63 |
| 7 | +++ | 20 |
| 9 | ++ | 18 |
| 10/1 | ++ | >100 |
| 10/2 | + | >100 |
| 10/3 | ++ | >100 |
| 100 | +++ | 50 |
| 102 | +++ | 17 |
| 102/1 | +++ | >100 |
| 102/2 | +++ | >100 |
| 110 | +++ | >100 |
| 110/1 | +++ | >100 |

EC₅₀ ranges for the SARS-CoV-2 assay as described herein: +++: <0.1 μM; ++: 0.1 μM to <1 μM; +: 1 μM to <50 μM; 0: ≥50 μM.

### Example 205: Antiviral Activity on RSV

One day before infection, 20,000 HEp-2 cells per well were seeded in a 96-well plate. Example 100 or DMSO vehicle controls were applied on cell monolayers at indicated concentrations and infected immediately after treatment with respiratory syncytial virus (RSV, A2 strain expressing eGFP, Long strain or RSV-B) at MOI 0.001. Cells were incubated for 45 h at 37° C. and 5% CO₂. RSV infection was quantified either by counting green fluorescent infected cells using CTL-Immunospot reader or by ICC staining using RSV-targeting antibody. Shown as mean values of triplicate wells normalized to the corresponding vehicle-treated controls ±SD. The 50% inhibitory concentrations (IC₅₀) were calculated using the nonlinear regression analysis in GraphPad Prism and was found to be below 50 nM in all three strains for representative Example 100.

### Example 206: Antiviral Activity on Influenza A

The assay for measuring influenza A has been described in general in Antimicrob. Agents Chemother. 2015; 59:2062 and applied to compounds of the present invention furnished the following representative results:

| Example # | EC₅₀ range | CC₅₀ [μM] |
|---|---|---|
| 100 | +++ | 82 |
| 102 | ++ | >100 |

EC₅₀ ranges for the SARS-CoV-2 assay as described herein: +++: <0.1 μM; ++: 0.1 μM to <1 μM.

1. A compound according to Formula (I): 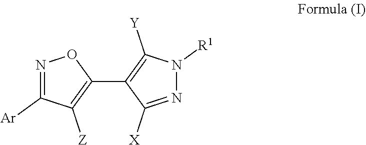
or a pharmaceutically acceptable salt, a solvate, a solvate of a salt, a hydrate or a polymorph thereof, wherein
Ar is selected from the group consisting of phenyl and heteroaryl, each of which is optionally substituted by one to five independently selected substituents R^{Ar}, having one or more hydrogen atoms optionally replaced by deuterium;
R^{Ar} is selected from the group consisting of halogen, —OH, —CN, alkoxy, haloalkoxy, alkyl, haloalkyl, mono- or dialkylamino-alkyl, mono- or di-alkylamino-alkoxy, —COOR′, —CONHR′, —CO—R′, —SO₂NHR′, —NH—CO—R′, —NO₂, —NH—SO₂—R′, —SO₂—R′, benzyloxy, —CO-heterocyclyl, —CO-cycloalkyl, —CONH-cycloalkyl, —CONH-heterocyclyl, —O-alkyl-heterocyclyl, —O-alkyl-cycloalkyl, (2-oxa-6-azaspiro[3.3]hept-6-yl)-C₁₋₄-alkoxy, amino, arylalkyl, cycloalkyl, heterocyclyl, phenyl and heteroaryl, wherein each of said alkoxy, arylalkyl, alkyl, cycloalkyl, heterocyclyl, phenyl and heteroaryl group is optionally substituted by one or more substituents independently selected from alkyl, haloalkyl, halogen and OH, having one or more hydrogen atoms optionally replaced by deuterium;
R′ is independently selected from the group consisting of H, OH, alkyl and haloalkyl, having one or more hydrogen atoms optionally replaced by deuterium;
Z is selected from the group consisting of H, halogen, —CO—R^{Z}, —CH₂—O—R^{Z}, —CO—CH₂—R^{Z}, —CO—CH₂—O—R^{Z}, —COOR^{Z}, —NHCO—R^{Z}, —CO—NHR^{Z}, —N(R^{Z})₂, —CN, —NHCOOR^{Z}, —SO₂—R^{Z}, —SO₂NHR^{Z}, -alkyl-O—R^{Z}, -alkyl-O-alkyl-O—R^{Z}, amino, alkyl, phenyl, heteroaryl, heterocyclyl and cycloalkyl, wherein each of said alkyl, phenyl, heteroaryl, heterocyclyl and cycloalkyl group is optionally substituted by one or more substituents independently selected from the group consisting of halogen, alkyl, alkoxy, haloalkyl, —COO-alkyl, OH and cycloalkyl, having one or more hydrogen atoms optionally replaced by deuterium;
R^{Z} is selected from the group consisting of H, halogen, —OH, alkyl, haloalkyl, cycloalkyl, heterocyclyl, phenyl and heteroaryl, having one or more hydrogen atoms optionally replaced by deuterium;
X is selected from H or D;
Y is selected from H, halogen, haloalkyl, alkyl, cycloalkyl, heterocyclyl or an alkylester, wherein each of said alkyl, cycloalkyl and heterocyclyl is optionally substituted by one or more substituents independently selected from the group consisting of halogen, —OH, —CN, alkyl, alkoxy, haloalkyl and O-haloalkyl, having one or more hydrogen atoms optionally replaced by deuterium;
R¹ is selected from aryl, heteroaryl, cycloalkyl, heterocyclyl or alkyl, which is optionally substituted by one to five substituents R″, having one or more hydrogen atoms optionally replaced by deuterium;
R″ is independently selected from H, —CO₂R′″, —CONHR′″, —CR′″O, —SO₂N(R′″)₂, —SO₂NHR′″, —NR′″—CO-haloalkyl, —NO₂, —NR′″—SO₂-haloalkyl, —NR′″—SO₂-alkyl, —SO₂-alkyl, —NR′″—CO-alkyl, —CN, alkyl, haloalkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl, having one or more hydrogen atoms optionally replaced by deuterium;
R′″ independently represents H, haloalkyl, hydroxyalkyl, amino, alkoxy, —N═C(R″)₂, —NR″—CO—R″, —CR′″O, —CO₂R″, alkyl, cycloalkyl, aryl, haloaryl, haloarylalkyl, heteroaryl, heterocyclyl, arylalkyl or aminoalkyl, which are optionally substituted by one or more substituents R″, having one or more hydrogen atoms optionally replaced by deuterium;
wherein the longest chain allowed in R¹ are three coupled substituents R″ and/or R′″,
or, alternatively R¹ is a group of the structure 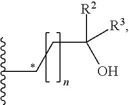
wherein
n is 0 or 1;
R² is H, deuterium, methyl or CD₃;
R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium; or
n is 1, R² is H, deuterium or methyl and R³ forms a methylene bridge to the carbon atom marked *, having one or more hydrogen atoms optionally replaced by deuterium;
and the hydrogen atom from the carbon atom marked * is substituted by deuterium;
provided, that at least one hydrogen in Formula (I) is replaced by deuterium with the proviso that the at least one deuterium is not contained in R²; and
provided, that the level of deuterium incorporation at each substituent designated as deuterium is at least 52.5%.

2. A compound according to claim 1, wherein
Ar is phenyl, which is substituted by one to five independently selected substituents R^{Ar}, having one or more hydrogen atoms optionally replaced by deuterium;
R^{Ar} is selected from the group consisting of F, Cl, Br, —OH, —CN, alkoxy, haloalkoxy, alkyl, haloalkyl, —O-alkyl-heterocyclyl, —O-alkyl-cycloalkyl, having one or more hydrogen atoms optionally replaced by deuterium;
X is H.

3. A compound according to claim 2,
wherein
Y is selected from haloalkyl, alkyl, cycloalkyl or heterocyclyl, wherein each of said alkyl, cycloalkyl and heterocyclyl is optionally substituted by one or more substituents independently selected from the group consisting of halogen, —OH, CN, alkyl, alkoxy, haloalkyl and O-haloalkyl, having one or more hydrogen atoms optionally replaced by deuterium.

4. A compound according to claim 3, wherein
Z is selected from the group consisting of —CO—R^{Z}, —CH₂—O—R^{Z}, —CO—CH₂—R^{Z}, —CO—CH₂—O—R^{Z}, —COOR^{Z}, -alkyl-O—R^{Z}, alkyl, phenyl, heteroaryl, heterocyclyl and cycloalkyl, wherein each of said alkyl, phenyl, heteroaryl, heterocyclyl and cycloalkyl group is optionally substituted by one or more substituents independently selected from the group consisting of halogen, alkyl, alkoxy, haloalkyl, —COO-alkyl, OH and cycloalkyl, having one or more hydrogen atoms optionally replaced by deuterium;
R^{Z} is selected from the group consisting of H, halogen, —OH, alkyl, haloalkyl, cycloalkyl, heterocyclyl, phenyl and heteroaryl, having one or more hydrogen atoms optionally replaced by deuterium.

5. A compound according to claim 4, wherein
R¹ is a group of the structure 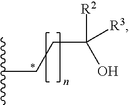
wherein
n is 0 or 1;
R² is H, deuterium, methyl or CD₃;
R³ is methyl, trifluoromethyl, ethyl, or taken with R² together forms a cyclopropyl group, having one or more hydrogen atoms optionally replaced by deuterium and the hydrogen atoms from the carbon atom marked * are substituted by deuterium;
or
n is 1, R² is H, deuterium or methyl and R³ forms a methylene bridge to the carbon atom marked *, having one or more hydrogen atoms optionally replaced by deuterium;
and the hydrogen atom from the carbon atom marked * is substituted by deuterium.

6. A compound according to claim 5, which is selected from 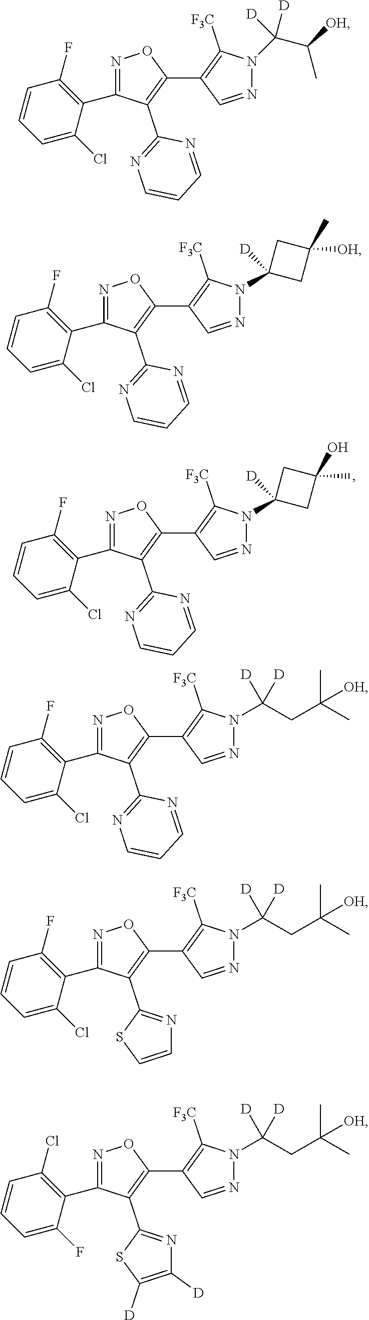 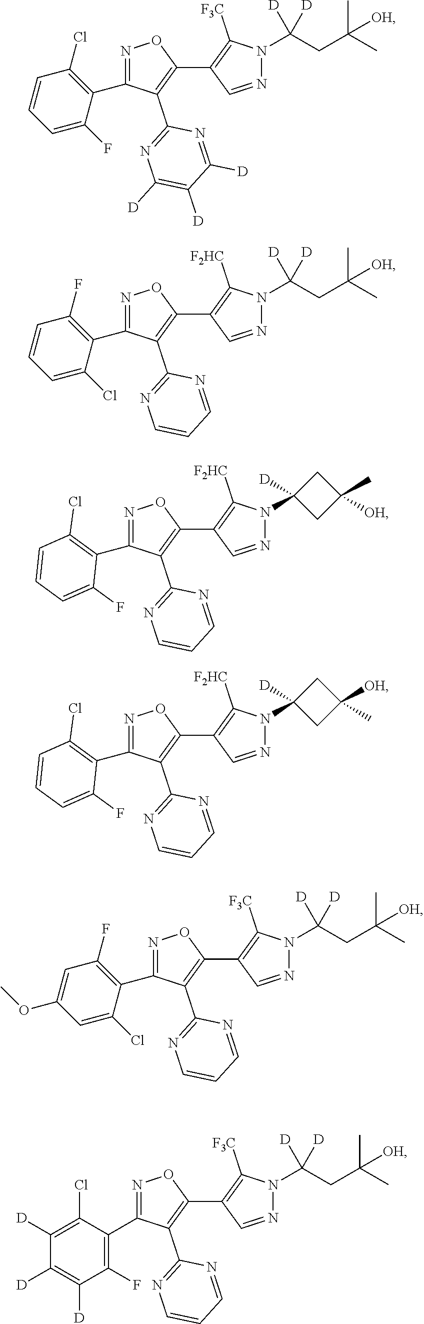 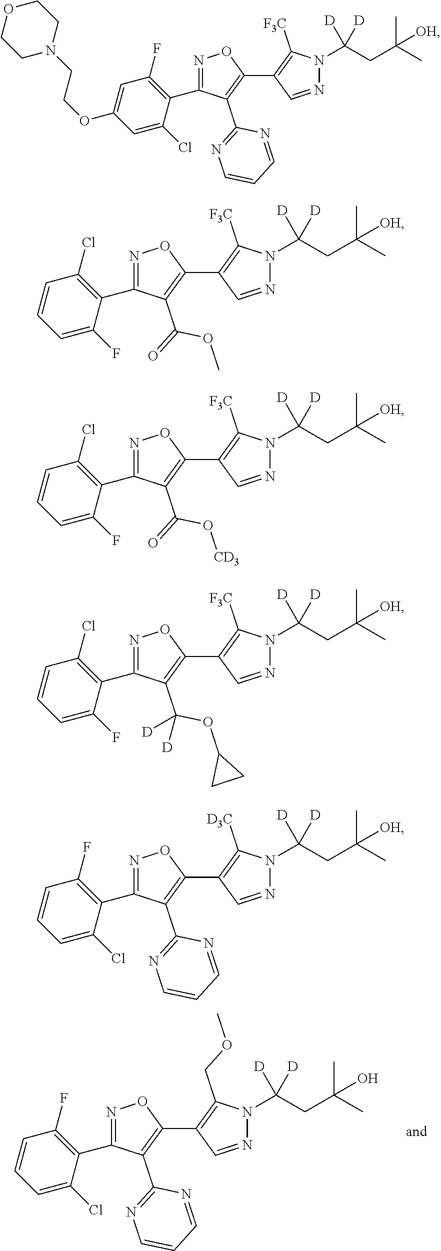 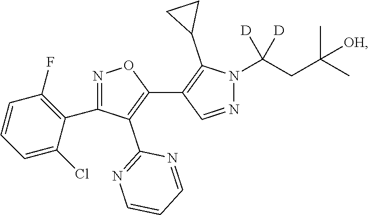 [SMILES: [2H]C([2H])(CC(C)(C)O)n1ncc(-c2onc(-c3c(F)cccc3Cl)c2-c2ncccn2)c1C1CC1]
or a pharmaceutically acceptable salt, a solvate, a solvate of a salt, a hydrate or a polymorph thereof.

7. A compound according to claim 4, wherein
R¹ is aryl or heteroaryl, which is optionally substituted by one to five substituents selected from the group consisting of F, Cl, Br, CN, —OH, alkyl, haloalkyl, —O-alkyl and —O-haloalkyl, having one or more hydrogen atoms optionally replaced by deuterium.

8. A compound according to claim 7, which is selected from 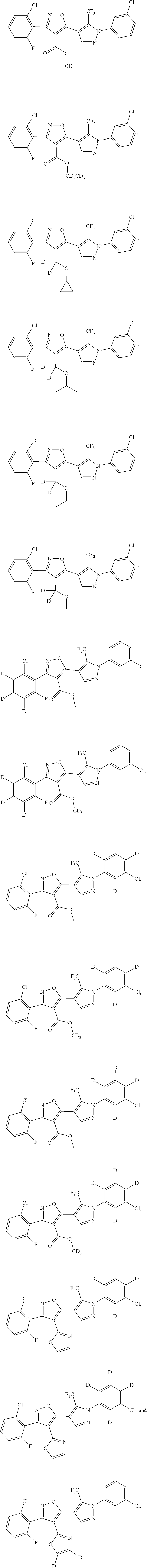
or a pharmaceutically acceptable salt, a solvate, a solvate of a salt, a hydrate or a polymorph thereof.

9. A method of treating a disease or medical condition in a patient in need thereof, comprising administering to the patient an effective amount of a compound according to claim 1 or a pharmaceutically acceptable salt, a solvate, a solvate of a salt, a hydrate or a polymorph thereof.

10. (canceled)

11. The method of claim 9, wherein treating the disease or medical condition comprises inverse agonism of RORγ/RORγt and/or inhibition of interleukin-17 (IL-17) and/or Interferon-γ (INF-γ).

12. A method of treating a disease or medical condition in a patient in need thereof, comprising administering to the patient an effective amount of a compound according to claim 1 or a pharmaceutically acceptable salt, a solvate, a solvate of a salt, a hydrate or a polymorph thereof, wherein the disease or medical condition is selected from the group consisting of psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behçet's disease, atopic dermatitis. Lichen planus, Sjögren's syndrome, spinal disc herniation, acne, graft-versus-host-reaction, host-versus-graft-reaction, AIM (autoimmune hepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, RSV and influenza, and cancer including leukemia, melanoma, lymphoma, carcinoma and sarcoma, especially prostate cancer including castration-resistant prostate cancer (CRPC).

13. A pharmaceutical composition comprising a compound according to claim 1 or a pharmaceutically acceptable salt, a solvate, a solvate of a salt, a hydrate or a polymorph thereof and a pharmaceutically acceptable carrier or excipient.

14. A method of treating a disease or medical condition in a patient in need thereof, comprising administering to the patient an effective amount of a compound according to claim 6 or a pharmaceutically acceptable salt, a solvate, a solvate of a salt, a hydrate or a polymorph thereof, wherein the disease or medical condition is selected from the group consisting of psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behçet's disease, atopic dermatitis. Lichen planus, Sjögren's syndrome, spinal disc herniation, acne, graft-versus-host-reaction, host-versus-graft-reaction, AIM (autoimmune hepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, RSV and influenza, and cancer including leukemia, melanoma, lymphoma, carcinoma and sarcoma, especially prostate cancer including castration-resistant prostate cancer (CRPC).

15. A method of treating a disease or medical condition in a patient in need thereof, comprising administering to the patient an effective amount of a compound according to claim 8 or a pharmaceutically acceptable salt, a solvate, a solvate of a salt, a hydrate or a polymorph thereof, wherein the disease or medical condition is selected from the group consisting of psoriasis, psoriatic arthritis, autoimmune thyroiditis, Grave's disease, rheumatoid arthritis, vitiligo, Crohn's disease, ulcerative colitis, inflammatory bowel disease, ankylosing spondylitis, diabetes type I, multiple sclerosis, celiac disease, systemic lupus erythematosus, uveitis, Behçet's disease, atopic dermatitis. Lichen planus, Sjögren's syndrome, spinal disc herniation, acne, graft-versus-host-reaction, host-versus-graft-reaction, AIM (autoimmune hepatitis), PBC (primary biliary cholangitis), PSC (primary sclerosing cholangitis), obesity, lupus nephritis, autoimmune thyroid disorders including graves disease and Hashimoto's disease, autoimmune uveitis, colitis, IMQ psoriasis, juvenile idiopathic arthritis, myasthenia gravis, systemic sclerosis, diabetes mellitus, osteoarthritis, infectious viral diseases including Covid-19, RSV and influenza, and cancer including leukemia, melanoma, lymphoma, carcinoma and sarcoma, especially prostate cancer including castration-resistant prostate cancer (CRPC).
